SEQUENCE LISTING (7 sequences)

SEQ ID NO: 1 - protein, 259 aa, synthetic construct
MSNPYQRGPNPTRSALTADGPFSVATYTVSRLSVSGFGGGVIYYPTGTSLTFGGIAMSPGYTADASSLAWLGRRLASHGFVVLVINTNSRFDGPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELCNASHIAPNSNNAAISVYTISWMKLWVDNDTRYRQFLCNVNDPALCDFRTNNRHCQ

SEQ ID NO: 2 - protein, 14 aa, synthetic construct
GKPIPNPLLGLDST

SEQ ID NO: 3 - protein, 15 aa, synthetic construct
GLNDIFEAQKIEWHE

SEQ ID NO: 4 - DNA, 56 bp, synthetic construct
  source 1..56: mol_type = other DNA
ctagtggtggaggaggctctggtggaggcggtagcggaggcggagggtcggctagc

SEQ ID NO: 5 - DNA, 56 bp, synthetic construct
  source 1..56: mol_type = other DNA
tatcagatctcgagctattacaagtcctcttcagaaataagcttttgttcggatcc

SEQ ID NO: 6 - DNA, 37 bp, synthetic construct
  source 1..37: mol_type = other DNA
caaggtctgcaggctagtggtggaggaggctctggtg

SEQ ID NO: 7 - DNA, 38 bp, synthetic construct
  source 1..38: mol_type = other DNA
ctacactgttgttatcagatctcgagctattacaagtc

(19) United States
(12) Patent Application Publication
(10) Pub. No.: US 2025/0163405 A1
(43) Pub. Date: May 22, 2025

(54) POLYMER-DEGRADING ENZYMES USING YEAST DISPLAY AND METHODS OF USE THEREOF

(71) Applicant: Wisconsin Alumni Research Foundation, Madison, WI (US)

(72) Inventors: Jeffrey Martell, Madison, WI (US); Mario Cribari, Aurora, OH (US); Maxwell Unger, Sussex, WI (US); Ashley Ogorek, Middleton, WI (US)

(21) Appl. No.: 18/954,282

(22) Filed: Nov. 20, 2024

Related U.S. Application Data
(60) Provisional application No. 63/601,011, filed on Nov. 20, 2023.

(51) Int. Cl. C12N 15/10; C12Q 1/44; G01N 33/542; G01N 33/573

(52) U.S. Cl. CPC C12N 15/1037; C12Q 1/44; G01N 33/542; G01N 33/573; C12Y 301/01074

(57) ABSTRACT

Provided herein are cells, compositions, and methods for identifying enzyme mutants having increased activity in cleaving synthetic polymers. The methods employ high-throughput cell surface display and directed evolution for rapidly assessing millions of enzyme mutants.

### CROSS-REFERENCE TO RELATED APPLICATIONS

This application claims the benefit of and priority to U.S. Provisional Application No. 63/601,011 filed on Nov. 20, 2023, the content of which is incorporated by reference in its entirety.

### FIELD OF INVENTION

The invention generally relates to a high-throughput surface display platform to evaluate enzyme mutants for increased activity in degrading synthetic polymers.

### REFERENCE TO AN ELECTRONIC SEQUENCE LISTING

The content of the electronic sequence listing (96029604657.xml; Size: 7,422 bytes; and Date of Creation: Nov. 18, 2024) is herein incorporated by reference in its entirety.

### BACKGROUND

Enzymes that degrade synthetic polymers have attracted intense interest for eco-friendly plastic recycling. However, because enzymes did not evolve for cleavage of abiotic polymers, directed evolution strategies are needed to enhance activity for plastic degradation. Previous directed evolution efforts relied on polymer degradation assays that were limited to screening ˜10⁴ mutants. High-throughput screening platforms that evaluate large variant libraries could greatly accelerate the discovery of plastic-degrading enzymes with both high thermostability and activity by identifying mutations that are challenging to find rationally. However, previous studies have been limited by relatively low throughput in the experimental evaluation of enzyme variants. We envisioned that yeast surface display coupled with fluorescence activated cell sorting (FACS) could enable the rapid testing of >10⁷ mutants, allowing for much larger libraries to be evaluated.

### SUMMARY

In an aspect, provided herein is a cell comprising: a cell anchoring protein associated with an outer surface of the cell; a nucleic acid sequence encoding a fusion protein within the cell, wherein the fusion protein comprises a candidate polymer-degrading enzyme and an anchoring sequence capable of associating with the cell anchoring protein; and a probe associated with the outer surface of the cell, wherein the probe comprises a polymer substrate and a detectable probe label. The fusion protein may further comprise a detectable fusion protein label. The detectable fusion protein label may be a myc tag, a spy tag, a snap tag, or a halo tag. The probe may be associated with a primary amine or a phenol on the outer surface of the cell. The polymer substrate may be positioned between a linker associated with the primary amine or the phenol and the detectable probe label.

The cell may further comprise the fusion protein, wherein the anchoring sequence is associated with the cell anchoring protein.

The candidate polymer-degrading enzyme may be leaf and branch compost cutinase (LCC) comprising an amino acid sequence having at least 90% identity to SEQ ID NO: 1. The LCC may comprise a H218 mutation. The LCC may comprise a H218Y, H218R, H218L, or H218N mutation. The LCC may comprise a Y61C mutation and a R151C mutation. The LCC may comprise a L117S and/or a C238R mutation. The LCC may comprise an A97V mutation.

The polymer substrate may comprise an ester bond. The polymer substrate may be an aromatic ester substrate, a lactic acid substrate, a polyamide substrate, a polyurethane substrate, and a polycarbonate substrate.

The cell anchoring protein is an Aga1P protein or a fragment thereof and the anchoring sequence may be an Aga2P protein or a fragment thereof.

The cell may be a yeast cell.

In another aspect, provided herein is a composition comprising the cell described herein and a first signal generator capable of binding with the detectable probe label. The composition may further comprise a second signal generator capable of binding with the detectable fusion protein label. The composition may further comprise an acidic media surrounding the cell. The acidic media may have a pH less than 4. The composition may comprise a heterogenous population of the cell, wherein the nucleic acid sequences encoding the fusion protein is different in each cell of the population; and wherein each fusion protein comprises a different candidate polymer-degrading enzyme.

In another aspect, provided herein is a method comprising: associating a probe with an outer surface of each of a plurality of cells, each probe comprising a polymer substrate and a detectable probe label, wherein each cell comprises: an cell anchoring protein associated with the outer surface of the cell, and a fusion protein comprising an anchoring sequence and a candidate polymer-degrading enzyme associated with the cell anchoring protein, wherein each cell comprises a different candidate polymer-degrading enzyme; and contacting the cells with a first signal generator capable of binding the detectable probe label; removing unbound first signal generator; and detecting a probe signal from the signal generator, wherein a reduced probe signal compared to a control probe signal from a cell that does not express the fusion protein indicates that the candidate polymer-degrading enzyme has polymer degrading activity.

The fusion protein may further comprise a detectable fusion protein label and the method further comprises: contacting the cells with a second signal generator capable of binding with the detectable fusion protein label; removing unbound second signal generator; and detecting a fusion protein signal from the second signal generator, wherein detected fusion protein signal indicates that the cell expresses the fusion protein. The method may further comprise isolating cells having reduced probe signal compared to the control probe signal. The method may further comprise sequencing the fusion protein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B. show strategies for directed evolution of polymer-degrading enzymes. A. Plate-based screening. Bacterial cells expressing enzyme mutants are grown in individual wells of multi-well plates. Enzyme activity is characterized in individual wells by addition of PET, followed by colorimetric assays or HPLC-based detection of monoaromatic products (TPA, MHET, and BHET). B. This work: ultrahigh-throughput yeast display evolution. A library of >10⁷ yeast clones is prepared in a single test tube, with each yeast cell displaying many copies of a distinct enzyme mutant. Cells are coated with a small molecule probe resembling PET. Cells displaying highly active variants cleave the probe, and probe cleavage is linked to a change in yeast cell fluorescence, allowing high-activity cells to be isolated using fluorescence activated cell sorting. Beneficial mutations are identified by subsequent DNA sequencing.

FIGS. 2A-2C show a yeast display platform for evolving PET-depolymerizing enzymes. A. Chemical structure of PET-mimicking probe assembled on yeast cell surface through a two-step reaction. The probe is tethered nonspecifically to endogenous lysines, and it consists of a terminal biotin moiety and a linker consisting of PEG₈, a triazole, and an aromatic ester. B. Yeast display selection procedure. Yeast cells are first coated nonspecifically with azides (via NHS labeling), followed by copper click to attach a probe containing an aromatic ester and a terminal biotin. High-activity mutants cleave the probe to a greater extent than low-activity mutants, leading to a difference in the amount of biotin displayed on each cell. Yeast are stained with a fluorescent streptavidin conjugate (sAv-PE) to detect biotin, and cells are sorted using FACS to isolate cells displaying low sAv-PE signal as a function of expression (which is detected by staining the myc epitope tag appended to the enzyme). C. Flow cytometry analysis of enzyme expression and activity on the yeast surface, before and after subsequent rounds of selection.

FIGS. 3A-3B show characterization of promising mutants identified from the yeast display selection. A. Computational model docking a PET tetramer substrate into the active site of LCC ICCG, based on the crystal structure of ICCG (PDB 7VVE).⁹ See Methods for details. H218 is shown in pink. The catalytic triad residues are shown as yellow sticks, and the PET tetramer is depicted as green sticks. The “wobbling” tryptophan (W190) is shown in light blue. B. Degradation of amorphous Gf-PET films at 70° C. using recombinantly purified LCC variants. Gf-PET film discs (8 mg) were incubated with 500 μM enzyme at 70° C. After 18 hours the concentration of monoaromatic products in the supernatant was quantified using HPLC (left-hand y-axis), and the mass of PET film remaining was measured (right-hand y-axis). Data represent the mean±standard deviation of three independent experiments.

FIGS. 4A-4C show characterization of PET-depolymerization by ICCG and ICCG (H218Y). A. Time course measurement of monoaromatic products released during depolymerization of amorphous PET by ICCG and ICCG (H218Y). Discs of amorphous Gf-PET film (8 mg) were incubated with 500 μM enzyme at 70° C. Aliquots of the supernatant solution were taken at the indicated time points, and monoaromatic products (TPA, MHET, and BHET) were quantified using HPLC. The concentration of products released by ICCG (H218Y) at 12 h (57.2 mM) corresponds to 99% depolymerization. Data represent the mean±standard deviation of three independent experiments. B. Pseudo-Michaelis-Menten (MM) analysis of rate of depolymerization of amorphous Gf-PET by ICCG and ICCG (H218Y). The indicated amounts of amorphous PET film were incubated with 100 nM enzyme at 70° C., and initial rates of monoaromatic product release were quantified using HPLC. Data points represent the mean±standard deviation of two replicates. The pseudo-K_{M} value for ICCG (H218Y) was 6.6 mg PET (4.0 to 10.4, 95% CI), and the pseudo-K_{M} value for ICCG was 16.8 mg PET (13.2 to 21.4, 95% CI). C. Depolymerization of 30% crystallinity PET powder by ICCG (H218Y) and ICCG. A solution of 1 μM enzyme was incubated at 70° C. with 8 mg of PET powder, and monoaromatic product in the supernatant was quantified using HPLC at the indicated time points. Data represent the mean±standard deviation of three independent experiments.

FIGS. 5A-5F show molecular dynamics simulations. A. Structural model for ICCG (H218Y) mutant, with a PET₄ oligomer substrate docked in the active site. The distance “d₁” between the catalytic serine (S165) and the nearest carbonyl carbon of PET₄ is indicated by a dashed line. B. Percentage of frames in the MD simulations in which the PET₄ substrate was dissociated from the active site, as defined by d₁>12 Å. The numbers shown represent the mean±standard deviation from bootstrapping the trajectories; see Methods section for details. C. Depiction of noncovalent interactions between the enzyme and PET₄ substrate in catalytically relevant conformations, with the catalytic serine close to a carbonyl carbon on PET, and the corresponding carbonyl oxygen hydrogen bonded to amide N—H groups in the oxyanion hole (Y95 and M166). D. Percentage of conformations that were catalytically relevant for ICCG and ICCG (H218Y). Catalytically-relevant conformations were defined based on two criteria, i) the catalytic serine within 3.5 Å of a PET carbonyl carbon, and ii) both amide N—H groups of Y95 and M166 within a threshold (ranging from 3 to 4 Å) of a PET carbonyl oxygen. The percentage of catalytically relevant conformation was calculated from the average of the percentages of frames over the range of distances in criterion ii). The numbers shown represent the mean±standard deviation from bootstrapping the trajectories; see Methods section for details. E. Depiction of Y218 (from the ICCG (H218Y) mutant) and H218 (from the parent ICCG enzyme) interacting with W190 in representative frames from the MD simulations. F. Histogram of distance between residue 218 (Y for the mutant, H for the parent ICCG enzyme) and W190, across all simulations (10×800 ns simulations for each enzyme).

FIGS. 6A-6E show flow cytometry data demonstrating lower limit of probe cleavage (PE signal loss). A. Uninduced cells, standard labeling. B. Uninduced cells with 10 μM exogeneous LCC ICCG added after CuAAC labeling. C. Induced cells expressing wild-type LCC with 10 μM exogenous LCC ICCG added after CuAAC labeling. D. Uninduced cells with no labeling. E. Median PE fluorescence intensity of indicated populations.

FIG. 7 shows BHET hydrolysis by pCTCON2-Aga2P-LCC containing yeast. Cells expressing Aga2P-LCC (induced sample) convert BHET into MHET, suggesting LCC is active on the yeast surface. Uninduced cells show no conversion above background level.

FIGS. 8A-8C show flow cytometry data of cells labeled with amide-containing probe 1. A. Round 3 sorted LCC mutant library population. B. wild-type LCC population. C. Histogram comparing populations. Minimal PE signal loss is observed in either population.

FIGS. 9A-9B shows flow cytometry data demonstrating signal difference between expressing and non-expressing cells after sorting. A. Histogram of induced round 3 sorted LCC library cells with full labeling procedure. Note that some portion of the population (˜25% in this case) does not express the Aga2P-LCC fusion construct when induced. B. Median PE fluorescence intensity of indicated population regions. The expressing population exhibits an approximately 30-fold reduction in PE signal.

FIG. 10 shows representative SDS-PAGE gel of LCC variant purifications. 12% SDS-PAGE gel visualized by Coomassie Brilliant Blue staining. LCC variants were purified as described in the Methods section. Lanes: (1) molecular weight standards, (2) LCC, (3) LCC (H218Y), (4) LCC (H218L), (5) LCC (H218N), (6) LCC (H218R), (7) LCC ICCG, (8) LCC ICCG (H218Y), (9) LCC ICCG (H218L), (10) LCC ICCG (H218N), (11) LCC ICCG (H218R).

FIG. 11 shows round 1-3 fluorescence activated cell sorting statistics.

FIGS. 12A-12C show A. DSC scan of amorphous PET film “Batch 1” (Goodfellow). Crystallinity was determined to be 3.35%. See method section on DSC above for further information. B. DSC scan of amorphous PET film “Batch 2” (Goodfellow). Crystallinity was determined to be 4.24%. See method section on DSC above for further information. C. DSC scan of semicrystalline PET powder (Goodfellow). Crystallinity was determined to be 30.29%.

FIG. 13 shows the individual product distribution of FIG. 3b. BHET values are too low to be visible with the y-axis presented. Reactions were run in triplicate. Data points represent the mean, and error bars correspond to the standard deviations.

FIG. 14 shows the inverse Michaelis-Menten plot. Initial rates were determined by measuring the amount of monoaromatic products (TPA, MHET, BHET) formed over 3 hours from “Batch 1” 8 mg PET film degradation reactions at 70° C. in 500 mM HEPES buffer, pH 8. Reactions were run in triplicate. Data points represent the mean, and error bars correspond to the standard deviations.

FIGS. 15A-15D show comparison of enzyme concentration effects among LCC variants. Data represent percent mass remaining of 24-hour 8 mg Gf-PET film (“Batch 2”) degradation at 70° C. in 500 mM HEPES buffer pH 8 with indicated enzyme concentration. Reactions were run in triplicate. The data presented represent the mean, and error bars correspond to the standard deviations. A. Composite plot depicting all variants at indicated concentrations (error bars not shown). B. Cross-sectional view of 1000 nM enzyme concentration. C. Cross-sectional view of 500 nM enzyme concentration. D. Cross-sectional view of 250 nM enzyme concentration.

FIG. 16 shows fit parameters for pseudo Michaelis-Menten experiment (FIG. 4B) from GraphPad Prism software.

FIG. 17 shows initial PET film degradation (“Batch 1”) at 70° C. in 500 mM acetate buffer pH 5.5. 1 μM of indicated enzyme was included. Initial rates were determined by measuring the amount of monoaromatic products (TPA, MHET, BHET) formed from 2 to 5 hours by HPLC. Reactions were run in triplicate. The data presented represent the mean, and error bars correspond to the standard deviations.

FIG. 18 shows BHET hydrolysis at 70° C., 500 mM HEPES pH 8 buffer. 50 nM of indicated enzyme was included. Initial rates determined by measuring the amount of MHET production over the first 30 minutes. Reactions were run in triplicate. The data presented represent the mean, and error bars correspond to the standard deviations. Note that some hydrolysis occurs in the absence of enzyme.

FIGS. 19A-19D show A. Definition of d1, d2, and d3. d1 is the minimum distance between S165 hydroxyl oxygen and C carbonyl of PET₄. d2 is the Minimum Distance between TYR95 amide nitrogen and any O carbonyl. d3 is the Minimum Distance between MET166 amide nitrogen and any O carbonyl. B. Histogram of d1 across all 10×800 ns unbiased MD simulations for both ICCG and ICCG (H218Y). C. Histogram of d2 (Å) across all 10×800 ns unbiased MD simulations for both ICCG and ICCG (H218Y). D. Histogram of d3 (Å) across all 10×800 ns unbiased MD simulations for both ICCG and ICCG (H218Y).

FIGS. 20A-20D show A. Average RMSF of the backbone atoms for each residue in ICCG or indicated variant MD simulations. B. Inset picture of a portion of FIG. 20A focusing on residue 187 to 193. C. Average RMSF of the side chain atoms for each residue in ICCG or indicated variant MD simulations. D. Inset picture of a portion of FIG. 20c focusing on residue 187 to 193.

FIG. 21 shows a histogram depicting the distance between any atom of position 218 in ICCG or indicated variant from any carbonyl oxygen atom in the PET₄ substrate, across all of the unbiased MD simulations (10×800 ns for each enzyme).

FIG. 22 shows a comparison between degradation activity on two different amorphous PET film batches (Goodfellow, ES30-FM-000145). PET film discs (8 mg) were incubated with either 500 or 1000 nM LCC ICCG at 70° C. After 18 hours the concentration of monoaromatic products in the supernatant was quantified using HPLC (left-hand y-axis), and the mass of PET film remaining was measured (right-hand y-axis). Reactions were run in duplicate. The data presented represent the mean, and error bars correspond to the standard deviations. A reduction in activity is evident for film batch 2 at both enzyme concentrations tested. Film batches were determined to be within 1% crystallinity of each other via DSC (FIG. 12).

FIG. 23 shows the percentage of conformations that were catalytically relevant for ICCG and related variants (expanded from FIG. 5). Catalytically-relevant conformations were defined based on 2 conditions, i) the catalytic serine is within 3.5 Å of a PET carbonyl carbon, and ii) both amide N—H groups of Y95 and M166 are within a threshold (ranging from 3 to 4 Å) of a PET carbonyl oxygen. The reported percentage of catalytically relevant conformations is the average of percentages of frames over the range of distances used in criteria ii). The numbers shown represent the mean±standard deviation from bootstrapping the trajectories; see Methods section for details.

FIGS. 24A-24B show A. Two hydrogen bond pairs form the catalytic triad of ICCG and related variants, between i. D210 and H242, ii. H242 and S165. B. Hydrogen bond occupancy between pairs of catalytic triad residues for ICCG and indicated variants, across all 10×800 ns unbiased MD simulations for both enzymes. The averages and error bars were obtained by bootstrapping.

FIGS. 25A-25D shows A. PyMOL image of LCC ICCG highlighting residues His191, Pro214, and His218 in the HIE protonation state (where only the ε-nitrogen is protonated). A potential hydrogen bond is shown between the His218 side chain and His191 backbone amide (PDB 7VVE). A simple computational model (not shown) with H218 in the HID protonation state (where only the δ-nitrogen is protonated) suggests that it could potentially hydrogen bond with carbonyl P214, but not simultaneously with H191. B. PyMOL image of LCC ICCG H218N highlighting residues Pro214, Asn218, and His19L. A pair of potential hydrogen bonds are shown between the Asn218 side chain and both the Pro214 and His191 backbone amides. C. Histogram of the shortest distance between Res218 sidechain and Pro214 backbone. D. The hydrogen bond occupancy between i) Res218 sidechain and His191 backbone (left), ii) Res218 sidechain and Pro214 backbone (right).

FIG. 26 shows a representative probe tethered nonspecifically to endogenous tyrosine residues which comprises a terminal biotin moiety and a linker consisting of PEG₈, a triazole, and an internal aromatic ester.

FIG. 27 shows flow cytometry analysis of round 3 sorted yeast enzyme expression and hydrolysis activity against FIG. 26 probe on the yeast surface for at a pH of a) 7.4, b) 6.2, c) 5.5, and d) 4.2. Enzyme expression is characterized on the x-axis by myc-epitope tag staining and activity is characterized on the y-axis via fluorescent streptavidin (phycoerythrin) labeling of biotins.

FIG. 28 shows the structure of a probe that may be tethered nonspecifically to tyrosines which comprises a terminal biotin moiety and a linker consisting of PEG₈, and an aromatic esters.

FIG. 29 shows a myc tag labeling and a spy, snap, and/or halo tag and optional myc tag labeling.

FIG. 30 shows flow cytometry analysis of enzyme expression labeling on the yeast surface with varied tags including a) SpyTag, b) HaloTag, and c) SnapTag all at pH 7.4

FIG. 31 shows flow cytometry analysis of SpyTag-displaying cells tagged with a SpyCatcher-Fluorescein conjugate at pH a) 7.4 and b) 4.

FIG. 32 shows flow cytometry analysis of a) ICCG H218Y-SpyTag-myc tag, b) ICCG H218Y-SnapTag-myc Tag, and c) ICCG H218Y-HaloTag-myc tag.

FIG. 33 shows enzyme expression of the full library (left) and enriched (sorted) library (right) following the third round of sorting. Enzyme expression is detected via the myc epitope tag labeled with fluorescent antibodies.

FIG. 34 illustrates an overview scheme of biotin-ester-triazole-phenol, first generation phenol probe.

FIGS. 35A-35C show second-generation yeast display platform. A. SpyCatcher-AlexaFluor 647 labels Spy-Tagged LCC mutants while the phenol-ester-biotin probe covalently attaches to cell surface tyrosine residues via oxidative HRP-catalyzed phenol labeling. B. Flow cytometry data of yeast expressing ICCG^{H218Y} and mutant libraries lead to cleavage of the probe at pH 4 as evidenced by the cells present in the purple “active” gate drawn. Activity of the parent ICCG^{H218Y} is shown as well as the initial library and rounds 1-4 sorted yeast, respectively, from left to right and top to bottom. C. Histogram showing sAv-PE fluorescence of round 4 sorted yeast compared to parent ICCG^{H218Y}. Post round 4 sorted yeast achieve the same level of sAv-PE fluorescence for the non-expressing population (right peak of trace), but lower PE signal is observed for the expressing post round 4 yeast (left peak of trace).

FIG. 36 shows evaluation of mutants from second-generation sorting campaign using SpyTag and biotin ester phenol probe. Monoclonal yeast populations were subjected to SpyCatcher-AlexaFluor 647 staining (x-axis) followed by HRP catalyzed phenol labeling with the biotin ester phenol probe (y-axis) and subsequent cleavage period and sAv-PE staining at pH 4. Purple “Active cells” gate shows cells that have the same sAv-PE fluorescence as unstained cells, indicating nearly complete cleavage of the probe for those cells.

FIGS. 37A-37B show activity of purified ICCG^{H218Y} and mutants towards solid PET film at 70° C. Degradation of amorphous Gf-PET films at 70° C. using recombinantly purified LCC variants. Gf-PET film discs (8 mg) were incubated with 500 nM enzyme at 70° C. A. After 16.5 h of degradation in 500 mM HEPES, the concentration of monoaromatic products in the supernatant was quantified using HPLC (left-hand y-axis), and the mass of PET film remaining was measured (right-hand y-axis). B. After 17 hours of degradation in deionized (DI) water (blue bars) or a 20 μM solution of TPA in DI water (pink bars), the concentration of monoaromatic products was quantified via HPLC.

FIG. 38 shows flow cytometry data on PET probe cleavage at pH 4 for the starting yeast library of ICCG+H218Y mutants (left) or monoclonal yeast displaying an evolved mutant (center).

FIGS. 39A-39C show activity of purified ICCG^{H218Y} and mutants at 55° C. against amorphous Gf-PET films at 55° C. using recombinantly purified LCC variants. Gf-PET film discs (8 mg) were incubated with 500 nM enzyme at 55° C. in designated solutions and after multiple time points the concentration of monoaromatic products in the supernatant was quantified using HPLC. A. 100 mM kPi buffer, pH 8 was used and time points were taken at 19.5, 28, 43, and 67.5 hours. B. 20 μM TPA solution, pH 4.2 was used and time points were taken at 18.5 and 38.75 hours. C. DI water was used as the solution and time points were taken at 18.5 and 38.75 hours.

FIGS. 40A-40E show sites of beneficial mutations identified from pH 4 evolution. A. Sites of beneficial mutations. B. Enriched active site mutation. C, D. A double mutant with a Cys pair. E. Preliminary structure modeling of the Cys pair mutant, assuming a disulfide is formed. Green: PET-like probe docked to ICCG+H218Y. Yellow: catalytic triad. Orange: mutations identified after sorting.

FIG. 41 shows the first-generation probe, biotin-ester-phenol, contained only one aromatic ester bond, shown in red, that was adjacent to a triazole ring and the second-generation probe, G2P, contains four possible aromatic ester bonds to be cleaved and is flanked by ethylene glycol units, which is more similar to the structure of PET. Both probes are attached to the cell surface via HRP-catalyzed oxidative phenol coupling with tyrosine residues on the cell surface.

FIGS. 42A-42B show A) an illustration of the assay design for probes with a polymer substrate for screening the candidate polymer degrading enzyme, and B) shows different general structures of polymer chains to incorporate into the probe (e.g. polyesters, polyamides, and polyurethanes).

### DETAILED DESCRIPTION

The present disclosure describes compositions and methods for identifying polymer-degrading enzymes using yeast display.

In a first aspect, provided herein is a cell comprising a cell anchoring protein associated with an outer surface of the cell; a nucleic acid sequence encoding a fusion protein within the cell, wherein the fusion protein comprises a candidate polymer-degrading enzyme and an anchoring sequence capable of associating with the cell anchoring protein localized to the cell surface; and an abiotic probe associated with the outer surface of the cell, wherein the probe comprises a polymer substrate and a detectable probe label. The cell is capable of expressing recombinant proteins on the outer cell surface. The cell may be a bacterial cell or a eukaryotic cell. In preferred embodiments, the cell is a yeast cell. The yeast cell may be an S. cerevisiae cell, a P. pastoris cell, or a Y. lipolytica cell. The outer surface of the cell refers to the cell membrane, wherein the proteins may be displayed on the outer surface of the membrane. The term “associated with” the cell surface, a protein, etc. refers to a physical connection thereto. “Within the cell” refers to the inner volume of the cell, e.g. the cytoplasm.

Candidate polymer-degrading enzymes include enzymes and enzyme variants that may have polymer-degrading function. The candidate polymer-degrading enzyme may have sequence identity with a known polymer-degrading enzyme. For example, the candidate polymer-degrading enzyme may have at least 70%, 80%, 81%, 82%, 83%, 84%, 85%, 86%, 87%, 88%, 89%, 90%, 91%, 92%, 93%, 94%, 95%, 96%, 97%, 98% or 99% identity with the known polymer-degrading enzyme. Polymer-degrading enzymes have the ability to degrade polymers or oligomers by catalyzing the cleavage of a bond joining monomers, such as C—O, C—N, or C—C bonds. An exemplary polymer-degrading enzyme is a polyethylene terephthalate (PET)-depolymerizing enzyme. In an exemplary embodiment, the polymer-degrading enzyme is a leaf and branch compost cutinase (LCC) or a variant thereof. The LCC enzyme may comprise the amino acid sequence MSNPYQRGPNPTRSALTADGPFSVATYTVSRLSVSGFGGGVIYYPTGTSLTFGGIAMSPG YTADASSLAWLGRRLASHGFVVLVINTNSRFDGPDSRASQLSAALNYLRTSSPSAVRAR LDANRLAVAGHSMGGGGTLRIAEQNPSLKAAVPLTPWHTDKTFNTSVPVLIVGAEADT VAPVSQHAIPFYQNLPSTTPKVYVELCNASHIAPNSNNAAISVYTISWMKLWVDNDTRY RQFLCNVNDPALCDFRTNNRHCQ (SEQ ID NO: 1) or a sequence having at least 70%, 80%, 81%, 82%, 83%, 84%, 85%, 86%, 87%, 88%, 89%, 90%, 91%, 92%, 93%, 94%, 95%, 96%, 97%, 98% or 99% identity thereto. The LCC enzyme may comprise 1, 2, 3, or 4 mutations relative to SEQ ID NO: 1. The LCC enzyme may comprise a mutation at H218 relative to SEQ ID NO: 1. The mutation may be a H218Y, H218R, H218L, or H218N mutation. The LCC enzyme may comprise a mutation at at least one of Y61 and R151 relative to SEQ ID NO: 1. The LCC enzyme may comprise a Y61C mutation and a R151C mutation.

The polymer-degrading enzyme or candidate polymer-degrading enzyme may be thermostable. A thermostable enzyme is one that has enzymatic function above 40° C. Suitably, thermostable enzymes have enzymatic function between 40° C. and 90° C. or 50° C. and 80° C.

The cell utilizes a display system for displaying the candidate polymer-degrading enzyme on the cell surface. The display system employs a cell anchoring protein associated with an outer surface of the cell and a second protein encoded by an anchoring sequence, where the second protein binds to the cell anchoring protein. By expressing a nucleic acid encoding a fusion protein comprising the second protein and the candidate polymer degrading enzyme as a fusion protein, the candidate polymer degrading enzyme is displayed on the cell surface. In exemplary embodiments, the display system is an a-agglutinin protein having two subunits, Aga1p and Aga2p. The cell anchoring protein is Aga1p and the second protein is Aga2p. Aga1p anchors Aga2p and the candidate polymer degrading enzyme onto the cell surface. Therefore, in some embodiments, the cell anchoring protein is an Aga1p protein or fragment thereof and the anchoring sequence encodes an Aga2p protein or a fragment thereof.

The fusion protein encoded by the nucleic acid sequence may further comprise a detectable label, termed herein as a detectable fusion protein label. The detectable fusion protein label may comprise a myc tag, a spy tag, a snap tag, a halo tag, a flag tag, a V5 tag, an AP tag, other epitope tags, or any other detectable protein label.

The terms “nucleic acid,” “nucleic acid sequence,” “polynucleotide,” and “polynucleotide sequence,” refer to a nucleotide, oligonucleotide, polynucleotide (which terms may be used interchangeably), or any fragment thereof. A “polynucleotide” may refer to a polydeoxyribonucleotide (containing 2-deoxy-D-ribose), a polyribonucleotide (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs. These phrases also refer to DNA or RNA of genomic, natural, or synthetic origin (which may be single-stranded or double-stranded and may represent the sense or the antisense strand).

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated. The term “promoter” refers to a cis-acting DNA sequence that directs RNA polymerase and other trans-acting transcription factors to initiate RNA transcription from the DNA template that includes the cis-acting DNA sequence.

The term “fusion protein” refers to a protein or polypeptide formed from the combination of two or more different proteins or protein fragments. The terms “peptide,” “polypeptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The terms polypeptide, peptide, and protein are also inclusive of modifications including, but not limited to, glycosylation, lipid attachment, sulfation, carboxylation, hydroxylation, ADP-ribosylation, and addition of other complex polysaccharides. The terms “residue” or “amino acid residue” or “amino acid” are used interchangeably to refer to an amino acid that is incorporated into a peptide, protein, or polypeptide. The amino acid may be a naturally occurring amino acid and, unless otherwise limited, may encompass non-natural analogues of natural amino acids that can function in a similar manner as naturally occurring amino acids.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 100, at least 150, at least 200, at least 250, at least 300, at least 350, at least 400, at least 450, at least 500, at least 550, at least 600, at least 650, or at least 700 contiguous amino acid residues; or a fragment of no more than 15, 20, 30, 40, 50, 100, 150, 200, 250, 300, 350, 400, 450, 500, 550, 600, 650, or 700 amino acid residues; or over a range bounded by any of these values (e.g., a range of 500-600 amino acid residues) Such lengths are exemplary only, and it is understood that any fragment length supported by the sequences shown herein, in the tables, figures or Sequence Listing, may be used to describe a length over which percentage identity may be measured.

The probe associated with the outer surface of the cell comprises a polymer substrate and a detectable probe label. The probe may be associated with the outer surface by specific or nonspecific, covalent or noncovalent binding. Exemplary embodiments include probes associated with proteins on the other surface of the cell. For example, the probe may be covalently bound to an amino acid. Exemplary amino acids capable of binding probes include those with amine or phenol moieties, such as lysine and tyrosine, respectively. Methods for associating probes with the outer surface of the cell are provided in the Examples.

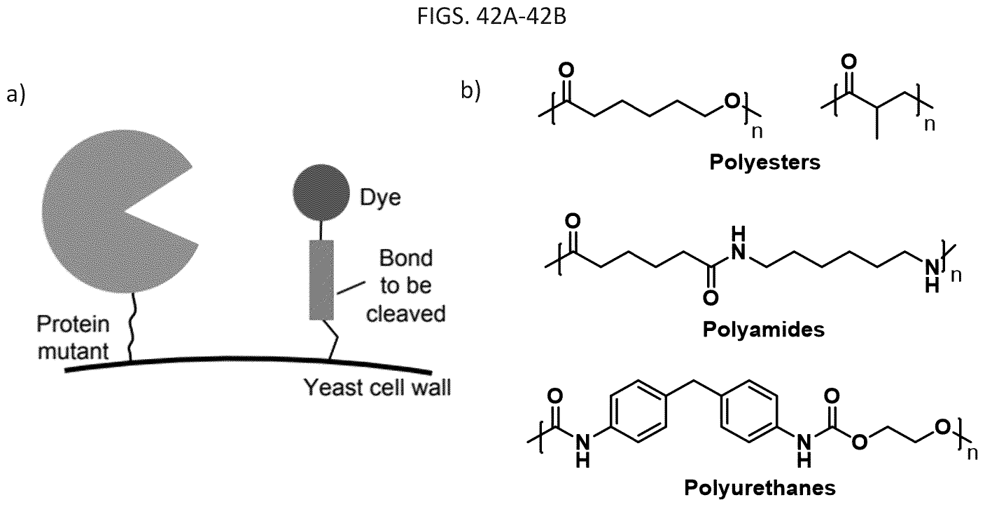

Probes comprise a polymer substrate for screening the candidate polymer degrading enzyme. The polymer-degrading enzyme or candidate polymer-degrading enzymes may be capable of catalyzing the degradation of a polymer substrate. Polymer substrates comprise one or more bonds that join monomers in a polymer, e.g., ester or amine bonds. Polymer substrates may comprise a fragment of a polymer, such as monomers, dimers, trimers, tetramers, oligomers (e.g., 5-20 monomers), or polymer fragments having more than 20 monomers. Exemplary polymer substrates include ester bonds, such as an aromatic ester substrate (e.g., a terephthalate substrate) or lactic acid substrate. Suitable polymer substrates also include polyamide substrates, polyurethane substrates, and polycarbonate substrates, as shown in FIG. 42.

The detectable probe label allows for interrogation of the integrity of the probe. As utilized in the Examples, the detectable probe label allows for determining candidate polymer-degrading proteins having polymer substrate degrading functionality. Suitably, the polymer substrate is positioned between the detectable probe label and the outer surface of the cell when the probe is associated with the cell. Exemplary detectable probe labels include biotin.

The probe may further comprise a linker between the polymer substrate and the outer surface of the cell (e.g. associated with the primary amine or phenol) when the probe is associate with the cell. Polyetheylene glycol (PEG) is an exemplary linker. The linker may be PEGₙ, wherein n is an integer. The integer can be any number from 2 to 10, e.g. at least 2, 3, 4, 5, 6, 7, 8, 9, or 10.

In a second aspect, disclosed herein is a library of cells comprising a heterogenous population of cells distinguished by nucleic acid sequences encoding for different fusion proteins. The fusion proteins may comprise different candidate polymer-degrading enzymes.

In a third aspect, disclosed herein is a composition comprising any of the cells described herein and a first signal generator capable of binding with the detectable probe label. The signal generator may be any molecule capable of emitting a detectable signal when bound to the detectable probe label. In some embodiments, the detectable probe label comprises biotin and the signal generator comprises a fluorescently labeled streptavidin.

The composition may further comprise a second signal generator capable of binding with the detectable fusion protein label. The detectable fusion probe label may comprise a myc tag, a spy tag, a snap tag, or a halo tag and the second signal generator may comprise a fluorescently labeled antibody, spycatcher fluorophore, fluorescently labeled benzylguanine, or halotag ligand fluorophore.

The composition may further comprise a media that the cell is cultured in. The media may have a pH from about 2 to about 9. In embodiments, the cell is in an acidic media. The acidic media may have a pH less than 6, e.g. a pH of 6, a pH of 5, a pH of 4, a pH of 3, a pH of 2. In embodiments, the pH is 4 or less.

The composition may comprise a heterogenous population of the cell described herein distinguished by nucleic acid sequences encoding for different fusion proteins. The different fusion proteins may comprise candidate polymer-degrading enzymes.

In a fourth aspect, provided herein is a method for preparing the cell described herein, the method comprising providing the cell comprising the anchoring protein associated with the outer surface of the cell and the nucleic acid sequence encoding the fusion protein within the cell; and associating the probe with the outer surface of the cell (e.g. with a primary amine or phenol on the outer surface of the cell). The fusion protein comprises the candidate polymer-degrading enzyme and the anchoring sequence capable of associating with the cell anchoring protein. The method may further comprise expressing the fusion protein. Expressing the fusion protein may comprise placing the cell under conditions in which gene expression can occur. The conditions may include temperature, pH, proteins and nutrients in the media, etc. The appropriate conditions can be readily ascertained by one of skill in the art. In some embodiments, providing the cell comprises selecting for a cell expressing the cell anchoring protein and inserting the nucleic acid sequence into the cell. The nucleic acid sequence may be inserted into the cell using any standard techniques known in the art, including but not limited to, transfection, transduction, and transformation.

The nucleic acid sequence may comprise an expression template, a translation template, or both an expression template and a translation template, including but not limited to plasmid DNA, linear DNA or mRNA. The template may comprise a construct configured to express the fusion protein. As used herein, the term “construct” refers to a recombinant polynucleotide, i.e., a polynucleotide that was formed artificially by combining at least two polynucleotide components from different sources (natural or synthetic). For example, the construct may comprise a polynucleotide encoding the fusion protein disclosed herein, operably linked to a promoter that (1) is associated with another gene found within the same genome, (2) from the genome of a different species, or (3) is synthetic. Constructs can be generated using conventional recombinant DNA methods. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. The composition may comprise one or more polymerases capable of generating a translation template from an expression template.

In some embodiments, associating the probe with the outer surface of the cell comprises associating the probe or a precursor thereof with a primary amine or a phenol on the outer surface of the cell. The amine may be a lysine or the phenol may be a tyrosine. In some embodiments, the probe precursor is associated with the amine and the polymer substrate and probe detectable label are conjugated to the probe precursor with a triazole. In other embodiments, the probe is associated with the phenol.

The probe-associated cell may be prepared in a composition comprising the cell and a first signal generator capable of binding with the detectable probe label. In some embodiments, the detectable probe label comprises biotin and the first signal generator comprises a fluorescently labeled strepdavidin. In some embodiments, the nucleic acid encodes a fusion protein comprising the candidate polymer-degrading enzyme, the anchoring sequence, and a detectable fusion protein label, wherein the composition further comprises a second signal generator capable of binding with the detectable fusion protein label. The detectable fusion probe label may comprise a myc tag, a spy tag, a snap tag, or a halo tag and the second signal generator capable of binding the detectable fusion protein label may comprise a fluorescently labeled antibody, spycatcher fluorophore, fluorescently labeled benzylguanine, or halotag ligand fluorophore. The composition may comprise an acidic media surrounding the cell, wherein the acidic media has a pH less than 6. In some embodiments, the acidic media has a pH of less than 4. The pH may be at most a pH of 6, a pH of 5, a pH of 4, a pH of 3, a pH of 2. In some embodiments, the composition comprises a heterogenous population of cells distinguished by nucleic acid sequences encoding for different fusion proteins, wherein each fusion protein has a different candidate polymer-degrading enzyme.

In a fifth aspect, provided herein is a method comprising associating a probe with an outer surface of a each of a plurality of cells, each probe comprising a polymer substrate and a detectable probe label, wherein each cell comprises at least one anchoring protein associated with the outer surface of the cell, and at least one fusion protein comprising an anchoring sequence and a candidate polymer-degrading enzyme associated with the cell anchoring protein, wherein each cell comprises a different candidate-polymer-degrading enzyme; and contacting the cells with a first signal generator capable of binding with the detectable probe label; removing unbound first signal generator; and detecting a probe signal from the first signal generator, wherein a reduced probe signal compared to a control probe signal from a cell that does not express the fusion protein indicates that the candidate polymer-degrading enzyme has polymer degrading activity. In some embodiments, the fusion protein further comprises a detectable fusion protein label and the method further comprises contacting the cells with a second signal generator capable of binding with the detectable fusion protein label, removing unbound second signal generator, and detecting a fusion protein signal from the second signal generator, wherein detected fusion protein signal indicates that the cell expresses the fusion protein.

The plurality of cells (e.g. library) may be prepared by inserting random mutations in the candidate polymer degrading enzyme nucleic acid sequence, and introducing these mutated nucleic acids into the cells. The mutations may be generated using error-probe PCR. Multiple rounds of the steps of contacting the cells with the first signal generator, removing unbound first signal generators, and detecting a probe signal from the signal generator may be performed to select for cells comprising having reduced probe signal, wherein in each round, cells that emit relatively high probe signal are discarded from the plurality of cells.

The method may further comprise isolating cells having reduced probe signal compared to the control probe signal. The method may further comprise characterizing the cells having reduced probe signal by examining their ability to degrade a film comprising the probe or the polymer substrate. The method may further comprise sequencing the fusion protein yielding the reduced probe signal or the nucleic acid encoding the fusion protein. The method may be performed with the cells described herein, the library of cells described herein, or the compositions described herein.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims.

Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

The following examples are meant only to be illustrative and are not meant as limitations on the scope of the invention or of the appended claims.

### Example 1

Enzymes that degrade synthetic polymers have attracted intense interest for eco-friendly plastic recycling. However, because enzymes did not evolve for cleavage of abiotic polymers, directed evolution strategies are needed to enhance activity for plastic degradation. Previous directed evolution efforts relied on polymer degradation assays that were limited to screening ˜10⁴ mutants. Here, we report a high-throughput yeast surface display platform to rapidly evaluate >10⁷ enzyme mutants for increased activity in cleaving synthetic polymers. In this platform, individual yeast cells display distinct mutants, and enzyme activity is detected by a change in fluorescence upon cleavage of a synthetic probe resembling a polymer of interest. Highly active mutants are isolated by fluorescence activated cell sorting and identified through DNA sequencing. To demonstrate this platform, we performed directed evolution of a polyethylene terephthalate (PET)-depolymerizing enzyme, leaf and branch compost cutinase (LCC). We identified activity-boosting mutations that substantially increased the kinetics of degradation for solid PET films. Biochemical assays and molecular dynamics (MD) simulations of the most active variant suggest that the H218Y mutation improves the binding of the enzyme to PET. Overall, this evolution platform increases the screening throughput of polymer-degrading enzymes by three orders of magnitude and identifies mutations that enhance kinetics for depolymerizing solid substrates.

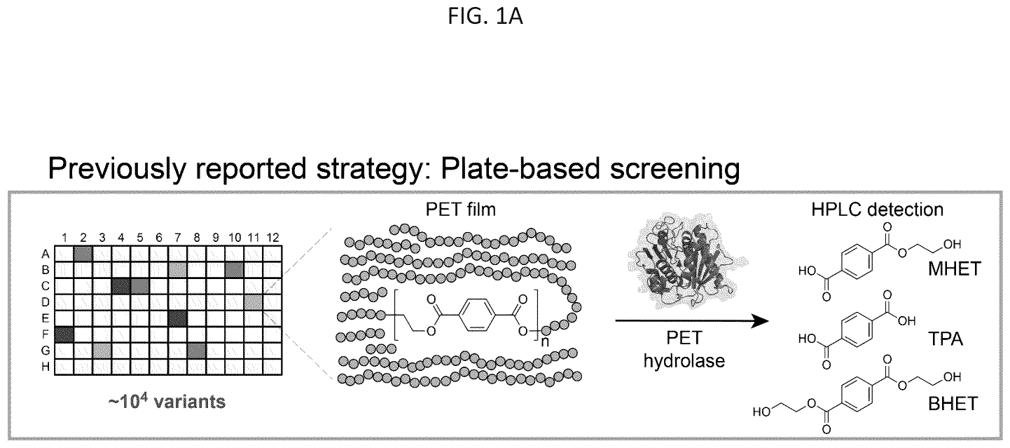

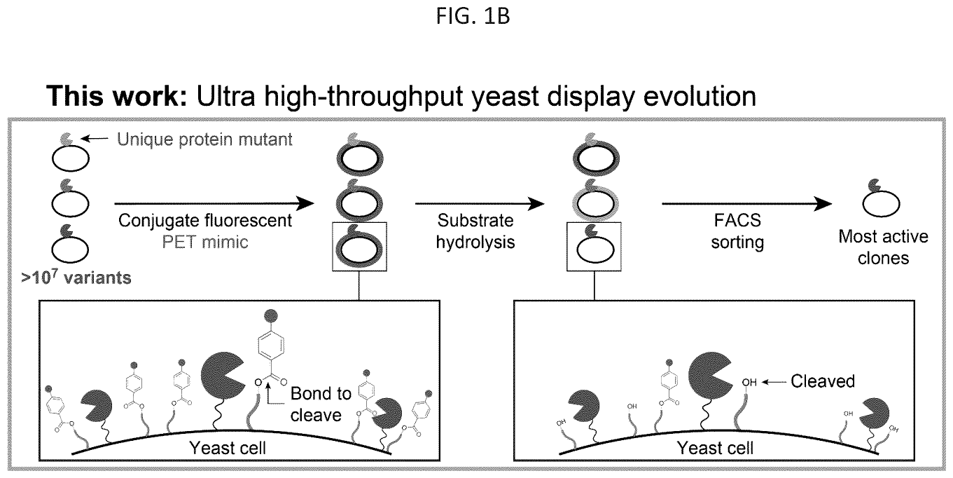

Overabundance of plastic waste has generated a global crisis, necessitating the urgent development of eco-friendly plastic recycling technologies. Enzymatic depolymerization of plastic waste would offer the advantages of mild reaction conditions and avoidance of toxic byproducts, leading to the production of constituent monomers that could be used to re-synthesize the plastic materials or diverted to microbes for biosynthesis of new compounds.¹ Promising progress has been made in harnessing enzymes for hydrolysis of the synthetic polyester polyethylene terephthalate (PET) into its constituent monomers, ethylene glycol and terephthalic acid (FIG. 1A). One prominent class of PET-depolymerizing enzymes is cutinases, which consist of a Ser-His-Asp catalytic triad and oxyanion hole that are typical of serine hydrolase enzymes.¹ Cutinases naturally degrade the waxy polyester cutin, but also exhibit PET-hydrolysis activity.^{2,3} Many cutinases, such as leaf and branch compost cutinase (LCC),⁴ are thermostable, which aids in their depolymerization of amorphous PET. They maintain activity above the glass transition temperature of PET (75° C. untreated),⁵ allowing them to operate on the polymer in a rubbery state in which chains are more sterically accessible compared to a glassy state.⁶ Another prominent class of PET-depolymerizing enzymes are PETases, which exhibit high structural homology to LCC. The initially reported enzyme in this family, Ideonella sakaiensis PETase (IsPETase), catalyzed release of monomers from PET films at 40° C., although the poor activity of IsPETase at higher temperatures limits the rate of bulk PET depolymerization.⁷

For enzymatic polymer recycling to be incorporated into process-scale plastic recycling, enzymes with high thermostability and rapid kinetics are needed. Since the initial discovery of PET-degrading enzymes, substantial progress has been made in improving their performance through rational protein engineering and directed evolution. The mutant enzyme LCC-ICCG, which exhibits an increase in Tₘ of 9.3° C. compared to wild-type LCC, was obtained by rational installation of a disulfide bond and site-saturation mutagenesis of residues near the active site.⁸ Efforts to further improve the thermostability of LCC-ICCG have resulted in variants such as ICCG_RIP and ICCG_I6M.^{9,10} These respective efforts employed rational design focused on improving internal hydrophobic interactions, or machine learning to predict residues influencing thermostability. In another example, in silico site-saturation mutagenesis was used to identify a double mutant of wild-type LCC with improved activity.¹¹ The PET-depolymerization activity of IsPETase has also been improved through directed evolution, resulting in engineered variants such as HotPETase, FAST-PETase, and DuraPETase.¹²⁻¹⁷ These variants exhibit greatly improved thermostability and PET-depolymerization kinetics relative to IsPETase. Beyond cutinases and PETases, new thermostable PET-depolymerizing enzymes are continuing to be uncovered,¹⁸⁻²⁰ generating new potential starting points for protein engineering and directed evolution campaigns. Despite these successes in improving PET-depolymerizing enzymes, there is an ongoing need to engineer new variants with both high thermostability and high activity. For example, FAST-PETase and HotPETase outperformed thermostable LCC variants for PET depolymerization at 50 and 60° C., respectively, indicating higher activity, but they degraded less PET than the LCC variants did at 70° C., indicating lower thermostability.^{12,13}

High-throughput screening platforms that evaluate large variant libraries could greatly accelerate the discovery of plastic-degrading enzymes with both high thermostability and activity by identifying mutations that are challenging to find rationally. However, previous studies have been constrained by relatively low throughput experimental evaluation of enzyme variants. Prior evolution campaigns screened mutants in a 96-well plate format using a colorimetric readout or high-performance liquid chromatography (HPLC) to monitor monomers released during PET plastic degradation (FIG. 1A).¹³⁻¹⁵ These methods were limited to evaluating 1.3×10⁴ variants due to the time and resources needed to analyze individual reactions.¹³ A major bottleneck was the need to grow cells expressing individual variants in multi-well plates, followed by assaying of enzymatic activity in each individual well. Computational design and machine learning approaches have shown promise in revealing which mutants to prioritize for testing, but previous computational design studies were accompanied by low-throughput testing to experimentally validate winners.^{12,14,21}

We envisioned that yeast surface display coupled with fluorescence activated cell sorting (FACS) could enable the rapid testing of >10⁷ mutants, allowing for much larger libraries to be evaluated. However, new methodology would need to be developed for this platform to be realized. Yeast-based evolution has been reported for proteases, but prior work involved bond cleavage in the endoplasmic reticulum, not on the cell surface, and employed a genetically encoded substrate rather than a synthetic polymer.²² Previous studies have used yeast display to evolve enzymes for improved recognition of non-genetically encoded substrates, but these enzymes catalyzed bondforming reactions, not bond-breaking reactions.²³⁻²⁵ To enable a yeast display platform for evolving polymer-cleaving enzymes, we hypothesized that surface-displayed enzyme mutants could be screened for activity based on their ability to cleave a synthetic probe that mimics the target polymer (FIG. 1B). Although small molecule probe cleavage on the yeast surface is a different context compared to degradation of bulk plastics, we hypothesized that this yeast display evolution platform would identify mutants exhibiting faster cleavage of the target polymer backbone while retaining their ability to degrade solid plastic substrates.

### Probe Design

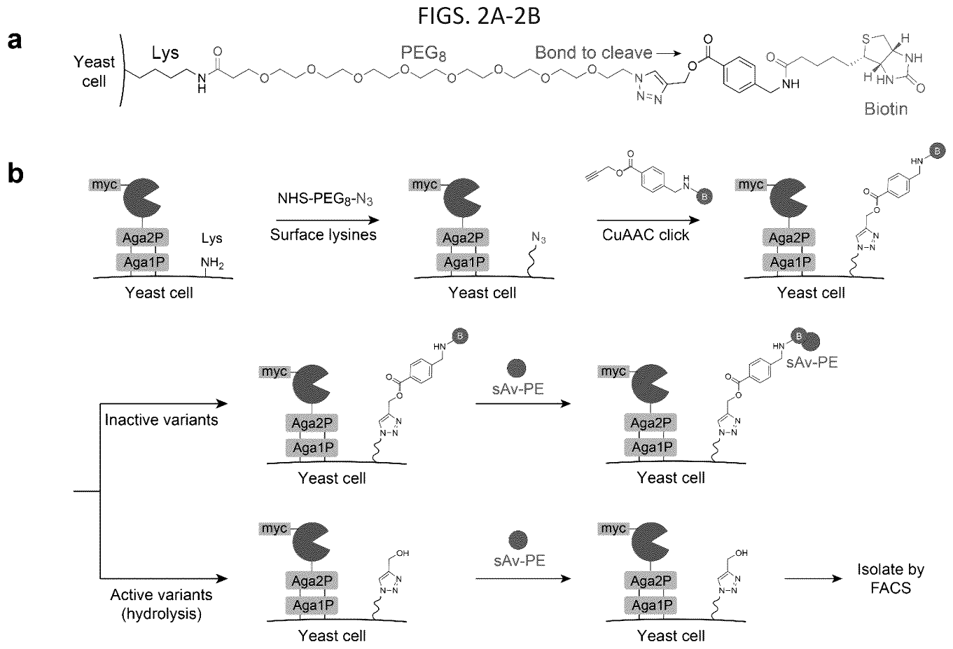

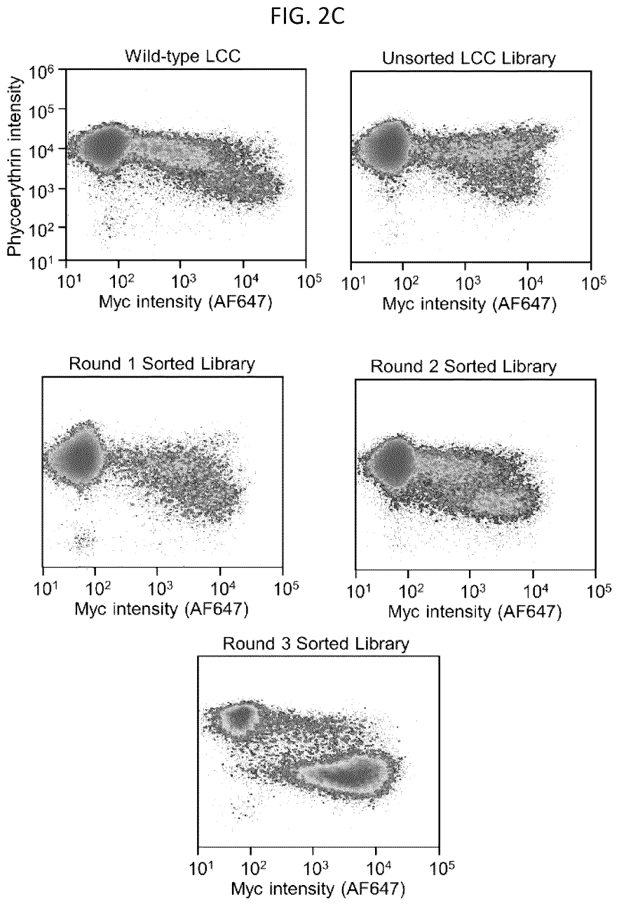

To enable detection of yeast surface-displayed enzymatic activity, we developed a strategy to coat the yeast surface with a PET-mimicking synthetic probe. The probe contains a terminal biotin, which is tethered to the yeast surface by a linker featuring a PET-resembling aromatic ester and a PEG₈ chain (FIG. 2A). Enzymatic cleavage of the probe leads to a change in yeast fluorescence (after streptavidin-fluorophore labeling) that can be used for fluorescence-activated cell sorting.

We designed a two-step labeling procedure to assemble this probe on the yeast surface (FIG. 2B). First, we first treated the yeast cells with NHS ester-PEG₈-N₃, which reacts with lysine sidechains and leads to non-specific tagging of endogenous proteins on the cell surface, thereby coating the yeast with —N₃ functional groups. Second, we used copper-catalyzed azide-alkyne cycloaddition (CuAAC) to functionalize the —N₃ groups with an aromatic ester-containing biotin probe. We hypothesized that the aromatic ester-containing probe would serve as a better mimic of PET than previously-employed nitro-phenolate-based probes, the hydrolysis of which correlates poorly with activity for PET plastic degradation.¹¹ Highly active surface-displayed enzymes would cleave the proximal aromatic ester chains, releasing biotin from the yeast surface.

Next, we introduced fluorescently labeled streptavidin (sAv-phycoerythrin, or sAv-PE), which binds to the unhydrolyzed biotinylated probe. Yeast cells displaying variants with low activity are labeled with sAv-PE, while yeast cells displaying highly active enzyme variants are poorly labeled due to loss of biotin via aromatic ester hydrolysis. Finally, fluorescence activated cell sorting (FACS) allows the isolation of cells displaying high activity relative to enzyme expression, detected by immunostaining of a myc epitope tag fused to the enzyme.

### Validation of the Evolution Platform

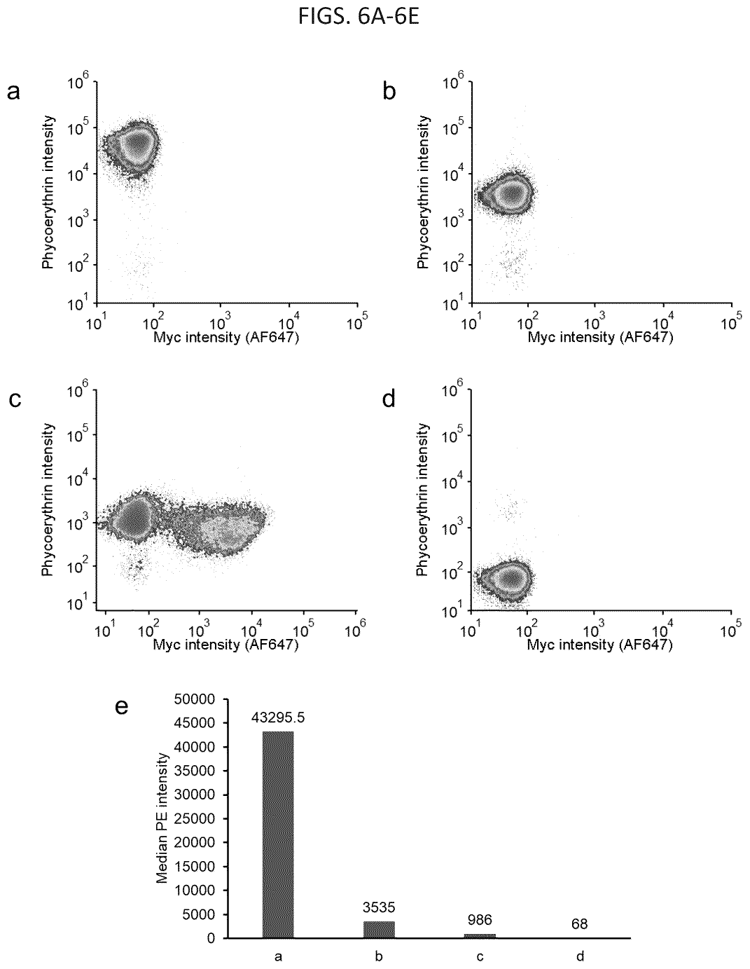

We validated the platform under desired evolution conditions. To increase the stringency of the selection, we performed the copper-click attachment under slightly acidic conditions (pH 5.5) where state-of-the-art PET-degrading enzymes are less active.¹ The CuAAC reaction has previously been reported to proceed under acidic conditions,²⁶ and has separately been shown to attach small molecules onto the yeast surface.²⁷ We confirmed strong biotin-labeling signal using this procedure, based on staining with sAv-PE followed by flow cytometry. The fluorescence of labeled cells was 637 times brighter than unlabeled cells, indicating a large potential dynamic range for the assay (FIG. 6). Negative controls in which the azide, alkyne, or copper were omitted did not exhibit fluorescence.

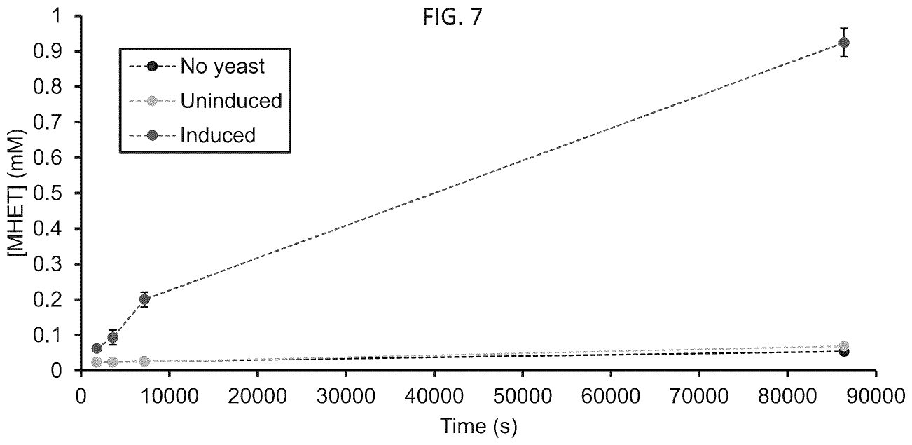

We assessed the activity of yeast surface-displayed LCC for hydrolyzing the surface-displayed probe. We selected LCC because it is a well-characterized PET-hydrolyzing enzyme that is active at 75° C., the glass transition temperature of PET.²⁸ We introduced into S. cerevisiae a plasmid encoding LCC fused to the C-terminus of the yeast mating protein Aga2P.²⁹ We confirmed that the displayed enzyme was active by hydrolysis of the exogenously added small molecule bis(2-hydroxyethyl terephthalate) (BHET), a partial degradation product that is released during PET hydrolysis and that is itself a substrate for LCC.³⁰ In contrast, yeast lacking surface-displayed LCC did not hydrolyze BHET (FIG. 7).

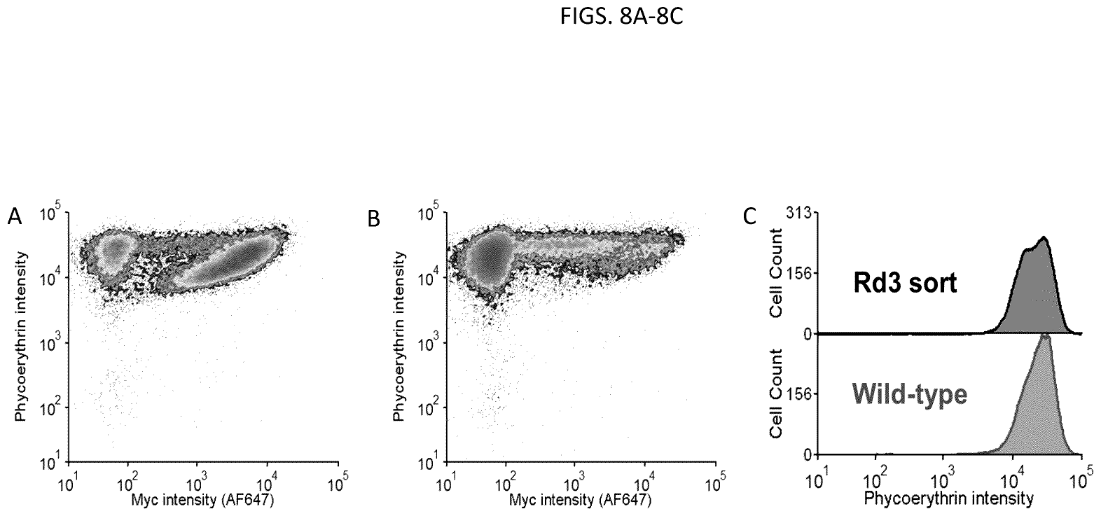

Next, we used flow cytometry to measure whether surface-displayed LCC led to cleavage of the aromatic ester probe attached to the yeast surface using the workflow shown in FIG. 2B. Cells with high myc signal, indicating high expression, had slightly decreased sAv-PE fluorescence, indicating aromatic ester cleavage (FIG. 2C). The correlation of sAv-PE loss with enzyme expression suggests minimal intercellular probe hydrolysis under these conditions, meaning the fluorescence of each individual yeast cell is an indicator of the activity of the enzyme mutant it is displaying. As expected, when purified LCC was added, the fluorescence of all cells decreased, regardless of whether they expressed enzyme on their surface (FIG. 6). The yeast-displayed probe contained two amide bonds (FIG. 2A), raising the possibility that the enzyme might be cleaving one of the amide bonds instead of the intended aromatic ester linkage. However, when a negative control probe was used that lacked the aromatic ester linkage but did contain two amides, sAv-PE fluorescence did not decrease as a function of enzyme expression, indicating that LCC does indeed cleave the intended aromatic ester bond (FIG. 8).

### Evolution of LCC

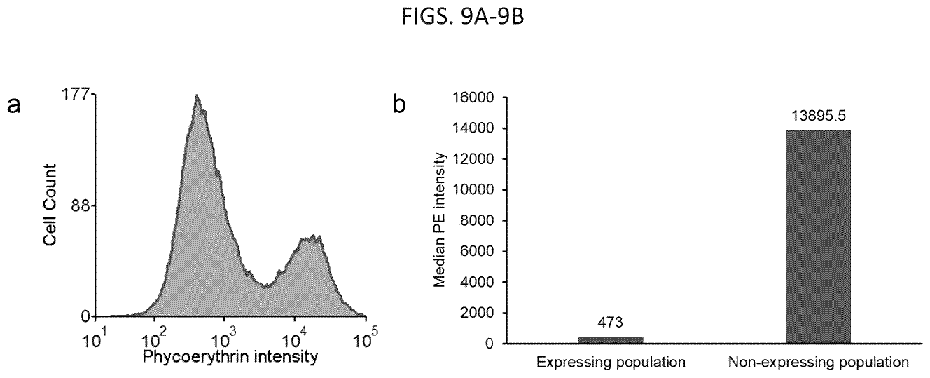

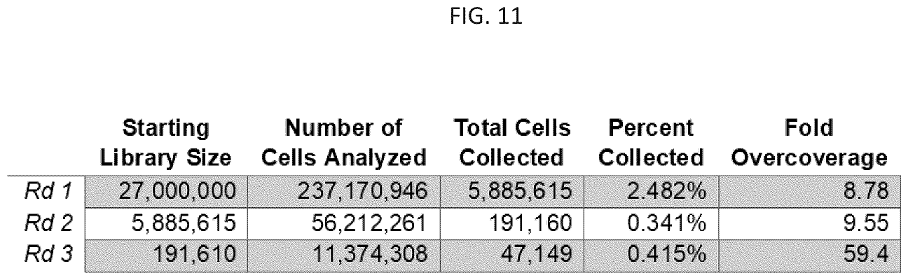

Having validated the platform design, we prepared a library of yeast cells with each displaying a distinct mutant of LCC. We prepared the library using error-prone PCR to install mutations randomly throughout the amino acid sequence. After electroporation of error-prone genes into yeast, we obtained a library consisting of 2.7×10⁷ clones, with an average mutation rate of 2.3 amino acids changed per gene. We subjected the yeast library to three successive rounds of labeling, sorting by FACS, and regrowth of the sorted cells prior to a subsequent round of labeling and sorting. Flow cytometry analysis of yeast fluorescence revealed increased activity (i.e. lower sAv-PE signal) for the myc-positive (i.e. enzyme-expressing) yeast with each successive round of sorting (FIG. 2C and FIG. 11). After the third round, a decrease of approximately 30-fold in sAv-PE fluorescence was observed for myc-positive cells (FIG. 9). Importantly, the yeast population post-sorting was incapable of cleaving the negative control probe lacking the aromatic ester, indicating that the selection had not converged on mutants that cleave amides (FIG. 8).

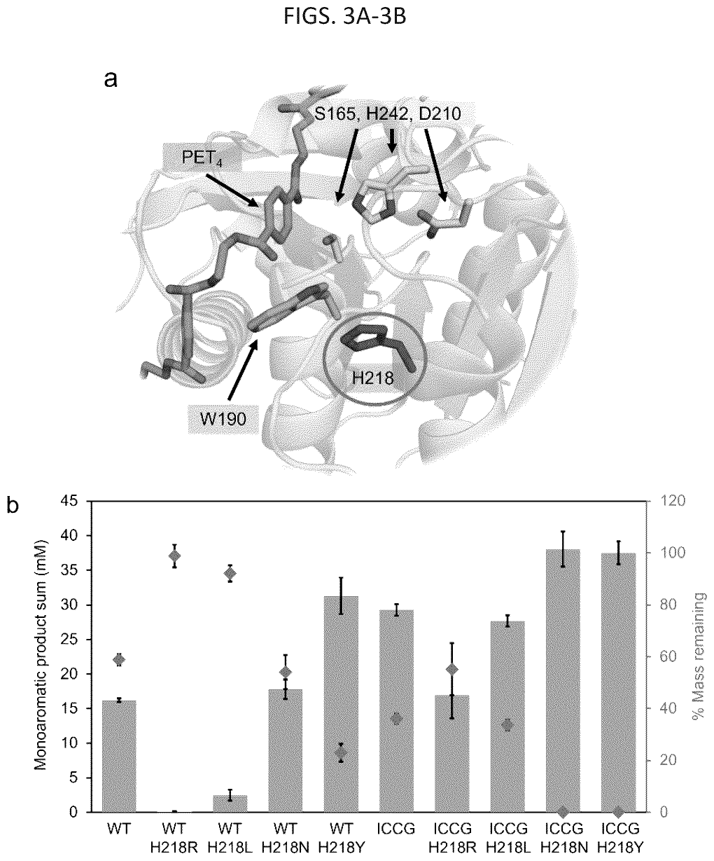

To assess whether the sorting had converged on promising mutations, we sequenced 77 clones remaining at the end of the third round of sorting. A diversity of mutations was observed. However, DNA sequencing revealed a clear enrichment of mutations at H218 (22% of the sequenced clones), with the H218Y, H218R, H218L, and H218N mutations all being observed. Based on the previously reported crystal structure of LCC,²⁸ H218 is a residue adjacent to W190, which forms a wall in the PET substrate binding pocket (FIG. 3A). W190 in LCC and the homologous Trp residue in PETase have been reported to be critical for PET hydrolysis activity.³¹ We therefore tested the H218 mutants for improved performance in depolymerizing PET plastic.

### Characterization of Evolved LCC Variants

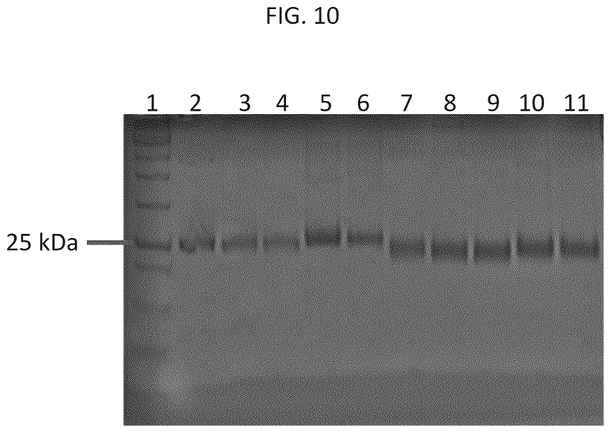

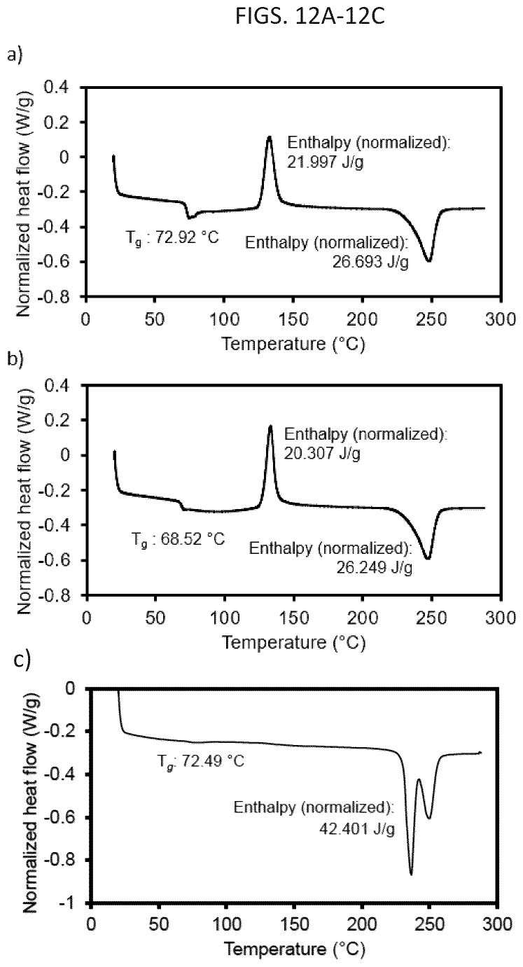

We cloned the LCC mutants H218N, H218R, H218L, and H218Y into a bacterial expression vector and purified them from E. coli (FIG. 10). Additionally, we cloned these mutations onto LCC “ICCG,” a quadruple mutant of LCC with improved thermostability.⁸ To assess whether the mutants were active in degradation of PET plastic, we tested the activity of each variant for depolymerization of PET films. We used a commercial low-crystallinity PET film (Gf-PET film, Goodfellow), which we determined to be 4.2% crystalline using differential scanning calorimetry (DSC) (FIG. 12B). Degradation of the films was assessed at 18 hours based on the change in film mass and the release of monoaromatic products, terephthalic acid (TPA), mono(2-hydroxyethyl)terephthalic acid (MHET), and BHET, using HPLC.^{11,32}

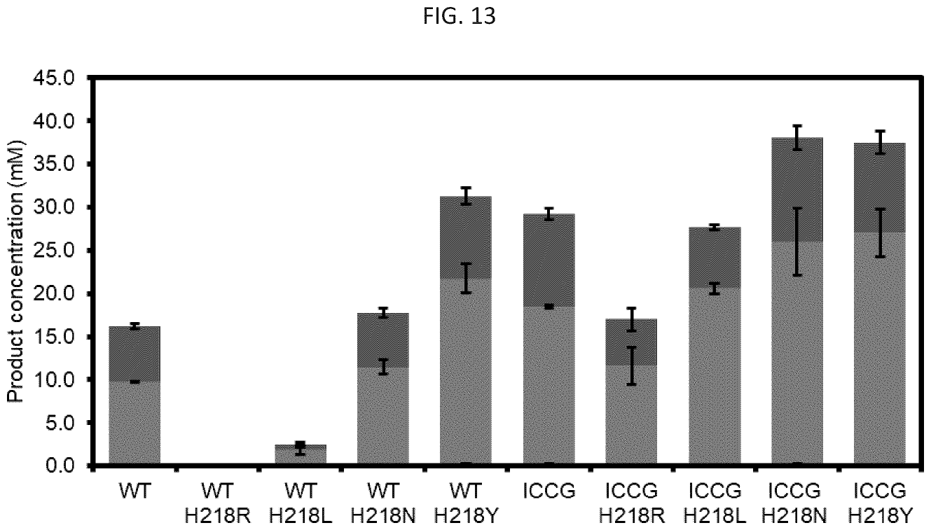

Compared to wild-type LCC, which exhibited ˜40% PET film degradation during the 18-hour experiment, the H218Y mutant exhibited ˜80% film degradation (FIG. 3B and FIG. 13). This 2-fold increase in PET degradation demonstrates the capability of our yeast display selection to converge on mutations that are beneficial not only on the yeast surface, but also in the context of PET plastic depolymerization. In contrast, LCC (H218N) performed similarly to LCC, while LCC (H218R) and LCC (H218L) exhibited little to no PET film degradation. We hypothesized that the poor PET film degradation performance of LCC (H218R) and LCC (H218L) may have arisen due to loss of thermostability, as the yeast display selection was performed at room temperature rather than 70° C. We therefore investigated whether introducing the thermostability-boosting “ICCG” mutations would enhance PET film degradation for the H218 mutants. The ICCG (H218Y) and ICCG (H218N) variants exhibited 100% PET film degradation, indicating a substantial improvement over the parent ICCG enzyme (˜60% PET film degradation). The ICCG (H218L) variant exhibited similar PET degradation to ICCG, suggesting that the stabilizing ICCG mutations may compensate for destabilization caused by the H218L mutation. The ICCG (H218R) variant gave ˜45% PET film degradation, which was slightly less than the parent ICCG enzyme, but a dramatic improvement compared to LCC (H218R), which showed no activity.

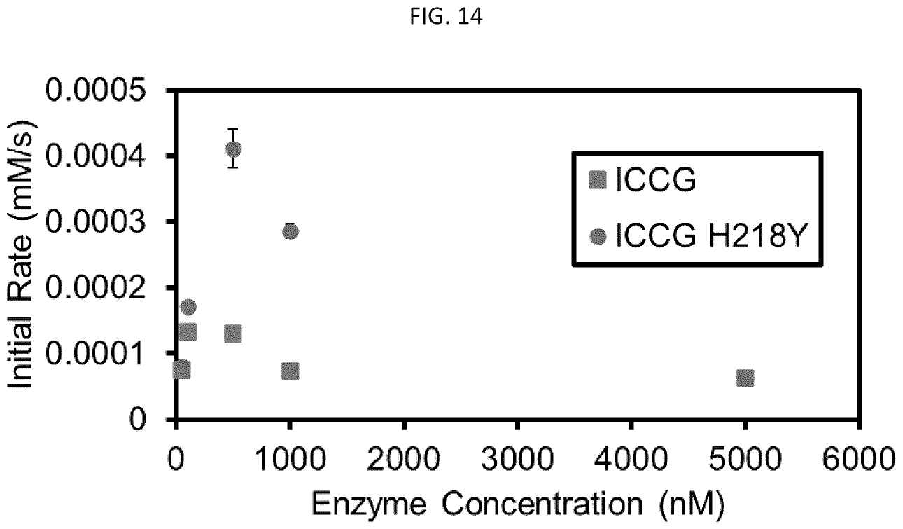

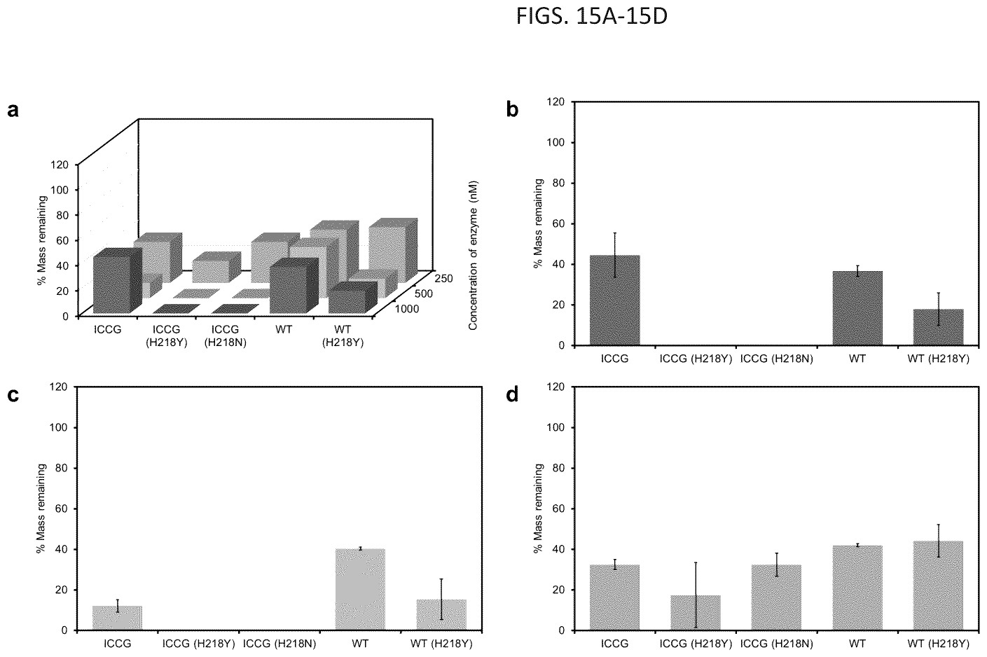

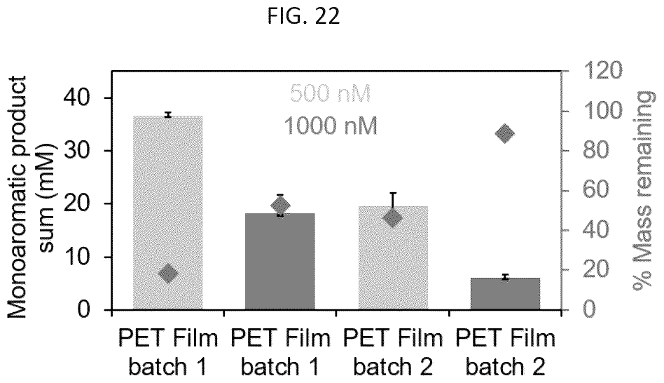

For the experiments depicted in FIG. 3B, an enzyme concentration of 500 nM was used, as we determined that using higher enzyme concentrations inhibited PET film degradation for some of the enzyme variants (FIG. 14 and FIG. 15). This inhibition at high enzyme concentrations is likely attributable to a molecular crowding effect, which has recently been reported in enzymatic PET depolymerizations.³³ Additionally, we observed that using two different Gf-PET films ordered from the same vendor (same catalog number, different lot number) led to different amounts of PET film degradation, potentially attributable to slightly different percent crystallinities (FIG. 12 and FIG. 22) or other differences in PET film morphology and surface composition, as one of the films had been stored under air for four years. These results are consistent with recent findings that the rate of enzymatic PET degradation is strongly dependent on PET morphology.¹⁸ Overall, our results underscore the importance of using matched PET source, enzyme concentration, and reaction conditions when comparing the activities of enzyme variants.

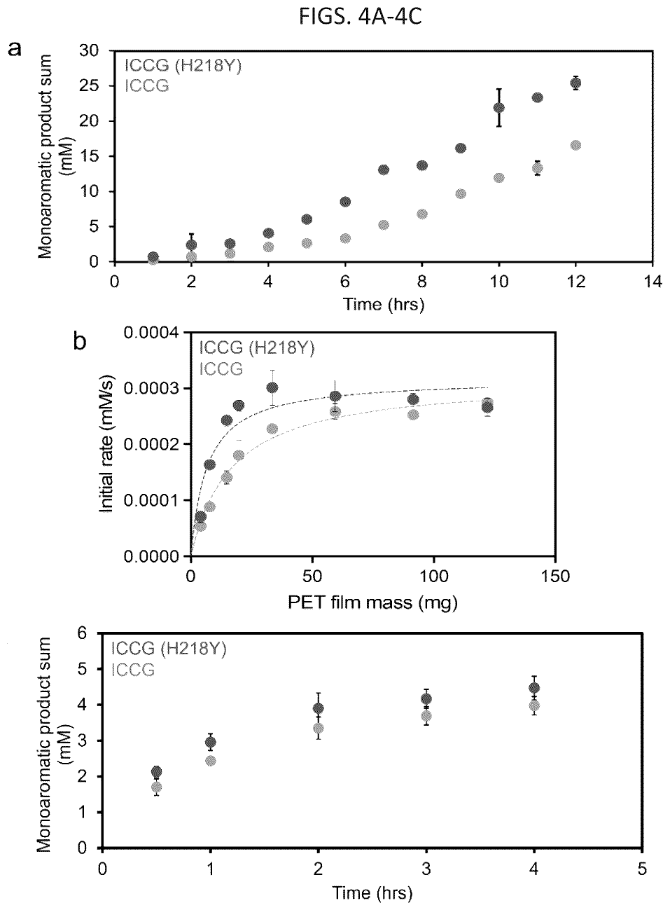

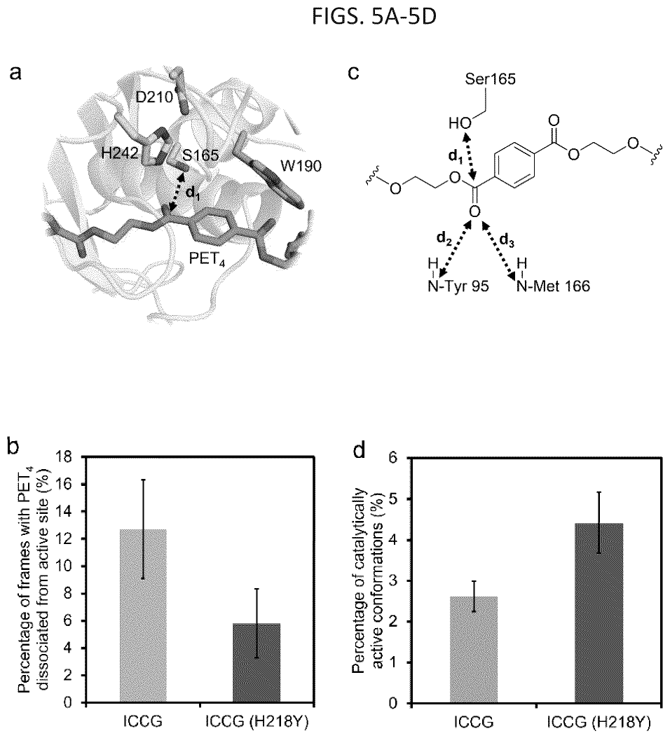

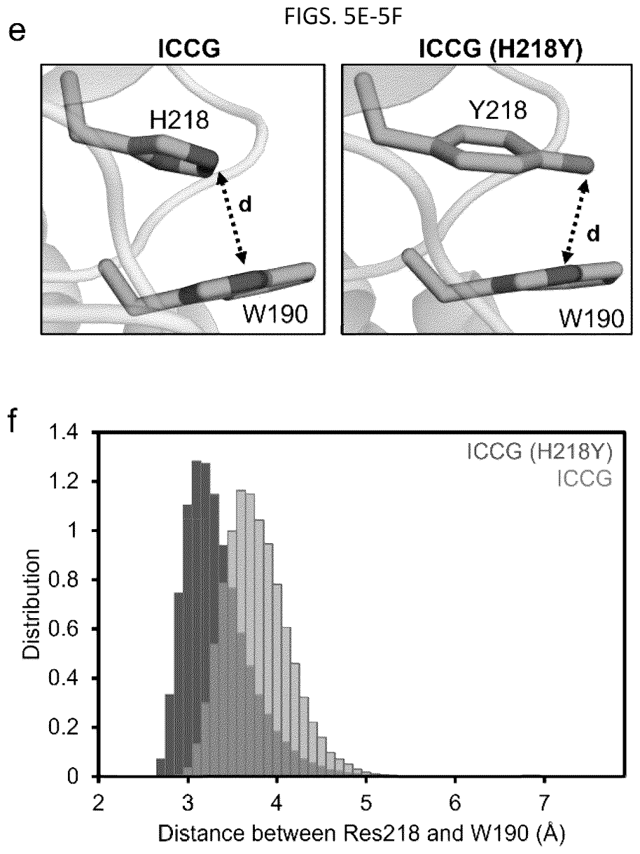

We selected ICCG (H218Y) for further characterization. We quantified the PET film degradation over time catalyzed by ICCG and ICCG (H218Y) (FIG. 4A). For both enzymes, a linear trend was established in the first 3 hours, followed by an increase in rate at 6 hours for ICCG and 3 hours for ICCG (H218Y). This increase in the rate of PET film degradation, which was also observed in previous studies,^{33,34} may be attributable to a change in the surface morphology of the film partway through the degradation, resulting in a substantial increase in the accessible PET surface area.³⁵ At 6 hours, ICCG (H218Y) generated 8.53 mM of released monoaromatic products, while the parent ICCG enzyme produced only 3.34 mM (2.6-fold lower). After this time point, the rate of product release from the parent ICCG enzyme increased, although the differential in monomer released between the two variants continued to grow throughout the 12-hour experiment.

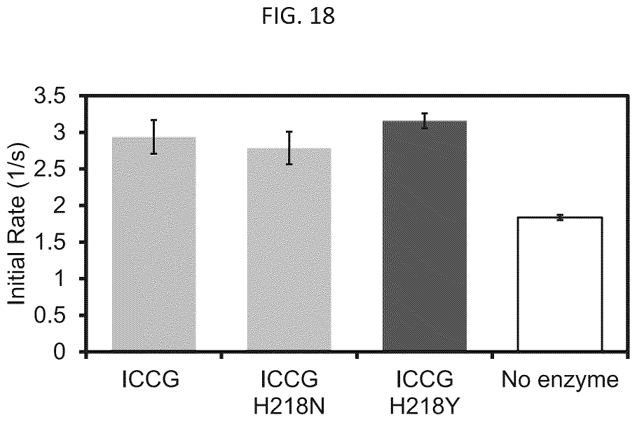

To assess whether the improvement from the H218Y mutation was due to general improved esterase activity, we measured the rate of BHET degradation for ICCG and the H218Y mutant, using identical temperature and reaction solution conditions as in the film degradations. Under these conditions, the enzymes exhibited similar initial rates of BHET degradation (FIG. 18), suggesting that ICCG (H218Y) was not enhanced in its activity as a general esterase.

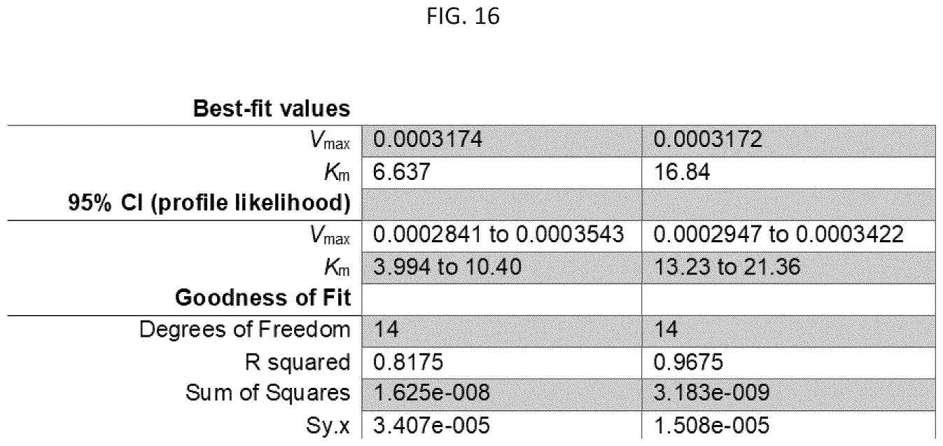

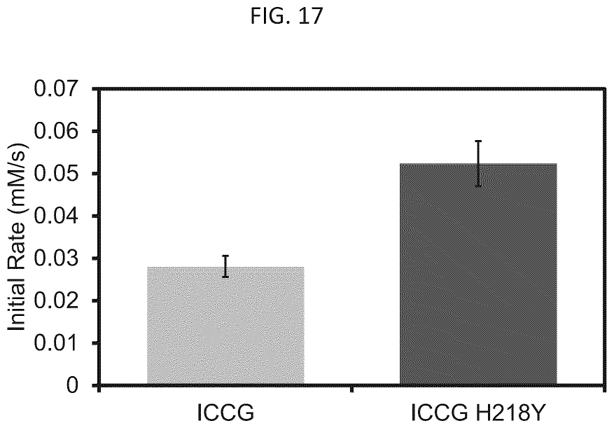

To investigate whether the improvement in PET film degradation was related to enzyme binding to the PET substrate, we tested a range of film and enzyme concentrations and determined a pseudo-Michaelis-Menten constant (K_{M}) (FIG. 4B and FIG. 16).³⁶⁻³⁷ Both the parent ICCG enzyme and the H218Y mutant exhibited a faster initial rate with increasing PET film loading, but the mutant exhibited a pseudo-K_{M} of 7 mg PET film, while the parent ICCG enzyme exhibited a pseudo-K_{M} that was >2-fold higher (17 mg PET film). The two enzymes exhibited similar Vₘₐₓ values, suggesting that the H218Y mutation enhanced PET-depolymerization specifically under conditions when the effective PET concentration is low. The improved activity afforded by the H218Y mutant was also observed during PET film degradation under the slightly acidic conditions (pH 5.5) used in the yeast display selection (FIG. 17).

We next characterized the effect of enzyme concentration on the initial rate of PET film degradation, to assess susceptibility to inhibition due to molecular crowding.³³ We tested the initial rate PET film hydrolysis, keeping film loading constant while varying enzyme concentration, in an inverse Michaelis-Menten analysis (FIG. 14).¹⁷⁻³⁶⁻³⁷ The H218Y mutant was more active at all enzyme concentrations tested. However, the difference was much more dramatic at higher enzyme concentrations (e.g. 500 nM and 1 μM). ICCG showed slower kinetics at 1 μM compared to 500 nM. At high enzyme loading (5 μM), both enzymes were inhibited. Collectively, these results suggest that the H218Y mutation may improve the resistance of ICCG to self-inhibition due to molecular crowding on the PET surface.³³ We also tested the rate of PET depolymerization using 30% crystallinity PET powder, as PET hydrolases can exhibit different activities depending on PET morphology.¹⁸ ICCG (H218Y) displayed an initial rate of product release of L2 mM/hour from the 30% crystallinity PET powder (FIG. 4c). The ICCG (H218Y) mutant exhibited slightly more depolymerization of crystalline powder compared to ICCG, although the improvement was much less pronounced compared to the PET film depolymerization experiments. Degradation of high crystallinity PET remains challenging in general for PET-depolymerizing enzymes due to the lower steric accessibility of these tightly packed polymer chains, although pre-treatments of crystalline materials or modification of reaction conditions have shown promise.³⁸

### Molecular Dynamics Simulations of ICCG (H218Y)

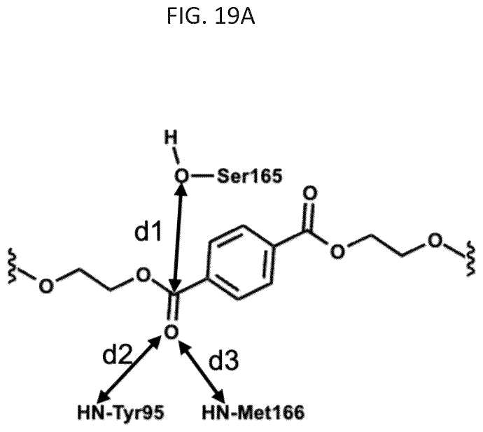

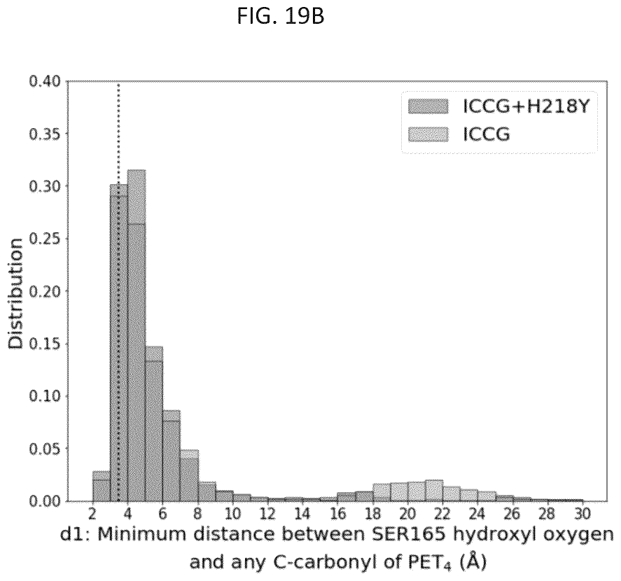

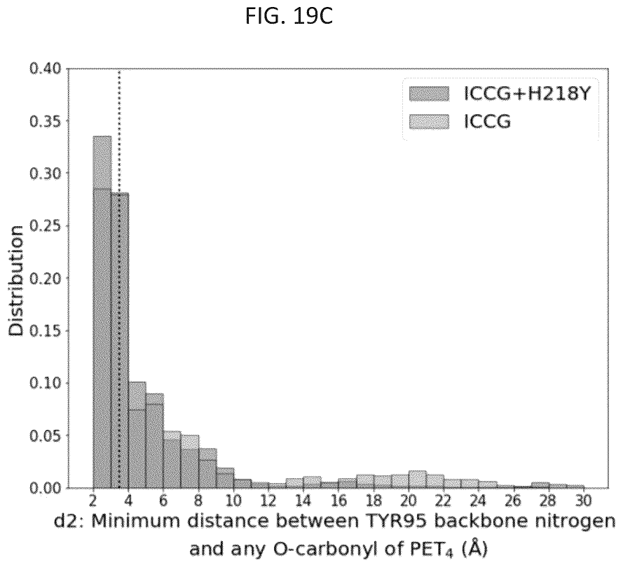

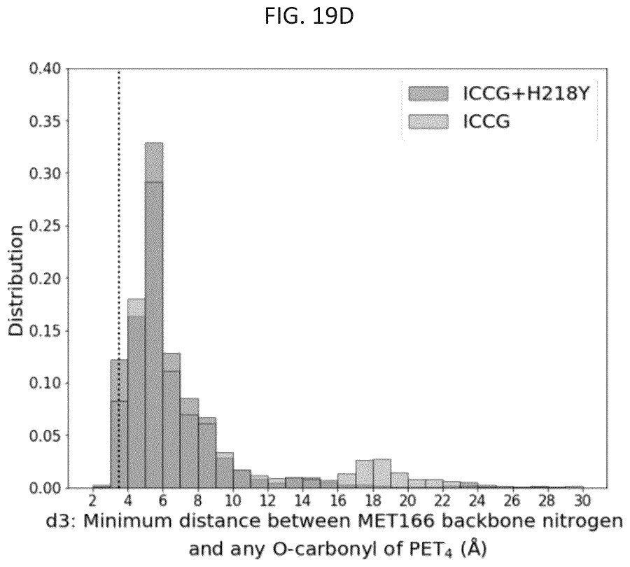

To gain insights into the molecular basis of improved PET depolymerization by the H218Y mutant, we performed molecular dynamics (MD) simulations. We first prepared structural models for a PET₄ tetramer docked into the active sites of ICCG and ICCG (H218Y), following a similar protocol as in a previous study (see Methods for details).¹⁰ In this structural model, one of the central PET₄ aromatic rings was positioned close to S165 (the catalytic serine), another was positioned near W190, and the other two were draped across the surface of the protein, extending toward D53 on the opposite side (FIG. 3A). The two docked structural models (one for each enzyme) served as initial structures for 10 unbiased MD simulations of 800 ns at 343 K, and the last 400 ns were used for the data analysis. The PET₄ substrate was highly dynamic during the simulations, with a high propensity to partially detach from the protein surface and curl onto itself in a conformation featuring intramolecular π-π stacking. Nonetheless, for most frames in the simulations for both enzymes, at least one aromatic monomer from the substrate was bound near the active site, based on the distance between S165 and a carbonyl carbon of PET₄ (FIG. 5A). However, during some of the frames for ICCG (˜13%), the PET₄ substrate was dissociated from the active site, as judged by a S165-PET₄ carbonyl carbon distance >12 Å (FIG. 5B and FIG. 19). In contrast, ICCG (H218Y) showed very few frames (<6%) with such a long distance, suggesting that ICCG (H218Y) may have a higher propensity to bind the PET₄ substrate in its active site.

We next quantified the percentage of catalytically relevant poses for both enzymes. The catalytically relevant poses were defined based on two criteria, a) the oxygen of S165 is within 3.5 Å of any carbonyl carbon of PET₄ and b) both nitrogens from the oxyanion hole residues (Y95 and M166) are within a threshold (ranging from 3 to 4 Å) of the carbonyl oxygen of criterion a) (FIG. 5C). The PET₄ substrate was in a catalytically relevant position for ˜4.4% of frames for the ICCG (H218Y) simulations, but only ˜2.6% of the frames for ICCG (FIG. 5D), suggesting that the H218Y mutation increases the propensity of PET₄ to bind in a catalytically productive pose.

To understand how the H218Y mutation improves enzyme-substrate binding, we examined its noncovalent interactions with the neighboring W190 residue. W190 forms the wall of the PET-binding cleft, and it has been previously called the “wobbling” tryptophan, owing to the hypothesis that conformational mobility of this Trp residue in IsPETase enhances catalytic activity.³⁹ IsPETase contains a Ser (S214) at the position homologous to H218 in LCC, and this Ser residue was hypothesized to increase conformational flexibility of W185 in IsPETase (homologous to W190 in LCC).³¹ Indeed, it has been shown that mutating H218 to Ser in LCC improves activity towards PET, albeit at a cost to thermostability.⁴⁰ On the other hand, during the directed evolution to develop HotPETase, the IsPETase mutation S214Y was introduced, and this activity-boosting Tyr mutation was reported to engage in π-π stacking with the key Trp residue, restricting its conformational mobility.¹³

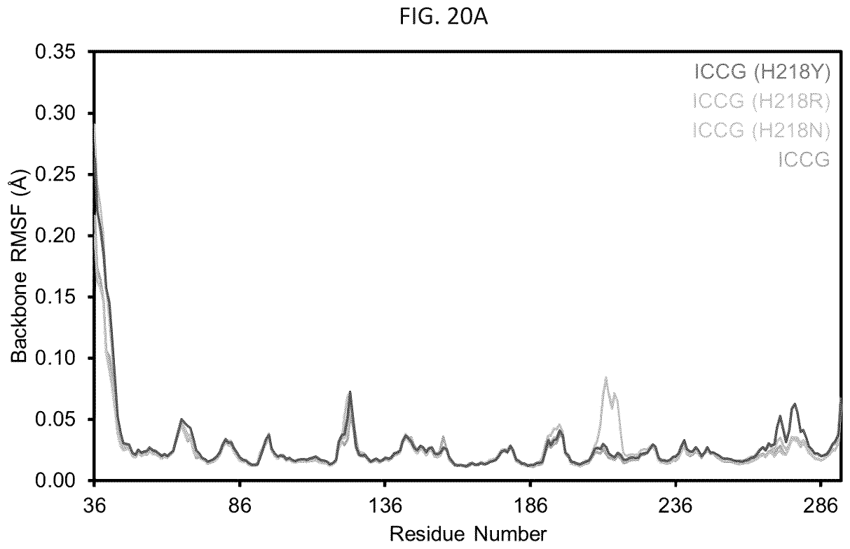

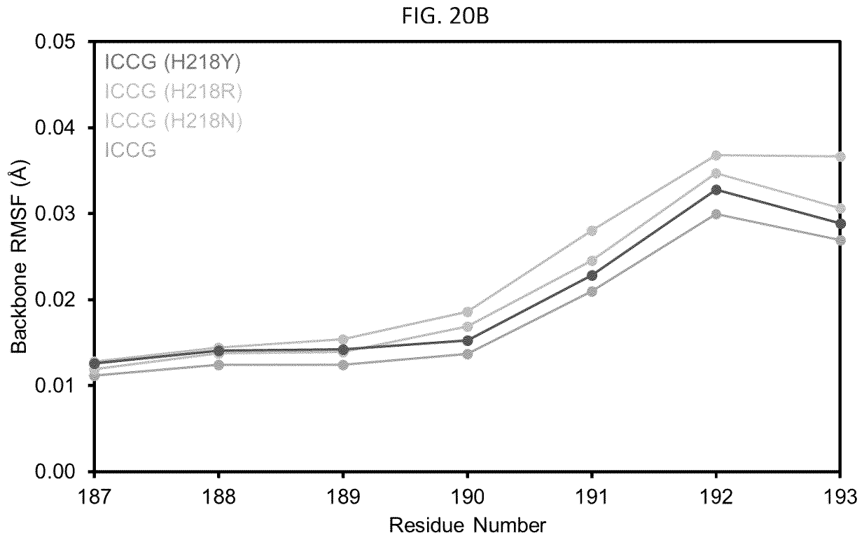

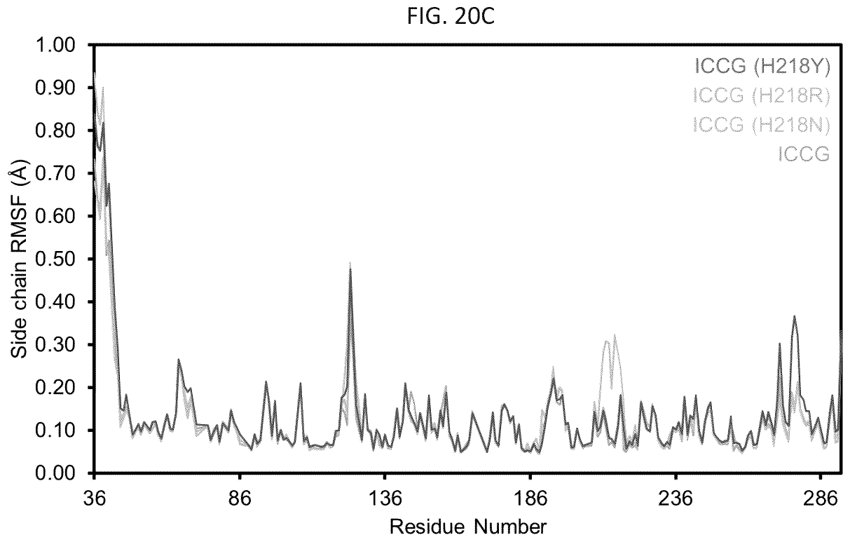

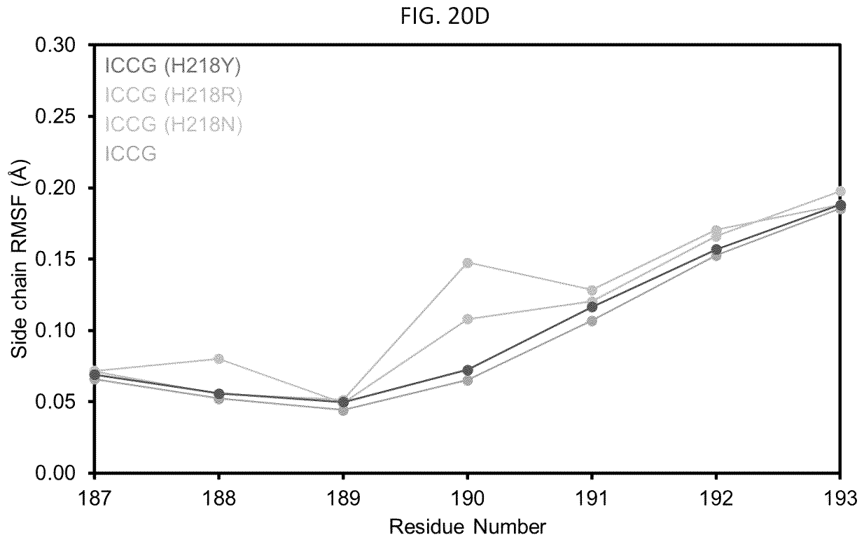

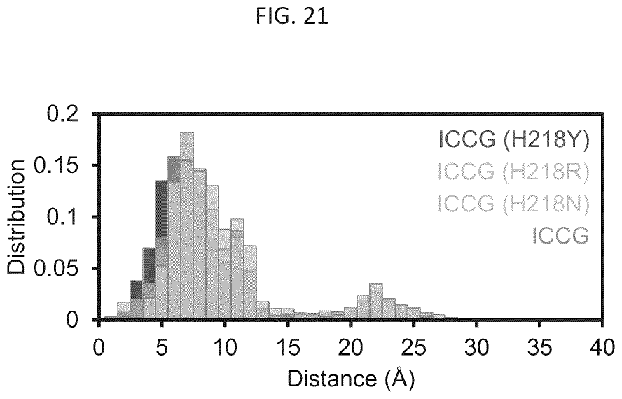

Throughout the MD simulations, both Y218 (in ICCG (H218Y)) and H218 (in ICCG) made close contact with W190 (FIGS. 5E and F). In both residues, the aromatic rings were suitably positioned to engage in π-π stacking with W190,⁴¹ but the Tyr O-Trp N distance in ICCG (H218Y) was shorter on average than the His N-Trp N distance in ICCG (FIG. 5F). In ICCG (H218Y), the phenol oxygen of Y218 is suitably positioned for hydrogen bonding with the indole N—H group of W190 (FIG. 5E). In contrast, H218 in ICCG is not as well-positioned to engage in hydrogen bonding. The RMSF values for W190 in ICCG (H218Y) and ICCG were very similar, suggesting that the H218Y does not affect the “wobbling” tendency of W190 (FIG. 20). It is possible that the noncovalent interactions between Y218 and W190 may subtly alter the positioning of W190—and thus the PET₄ substrate—relative to the catalytic triad. Additionally, noncovalent interactions of Y218 directly with the PET₄ substrate may contribute to the improved substrate binding, as the phenol oxygen of Y218 was much more likely to be within 5 Å of an oxygen in PET₄ than H218 was in the ICCG simulations (FIG. 21).

The apparent role of π-π stacking for the interactions of Y218 with W190 raises the question of whether the H218W and H218F mutations might also boost activity. In a recent report that identified activity-boosting mutations computationally,²¹ ICCG (H218W) and ICCG (H218F) exhibited very similar depolymerization of PET powder compared to the parent ICCG enzyme, but worse PET depolymerization than ICCG (H218Y). These results suggest that the π-π stacking of H218Y with W190 may not be solely responsible for improved PET binding; noncovalent interactions involving the phenol O—H group are likely also important.

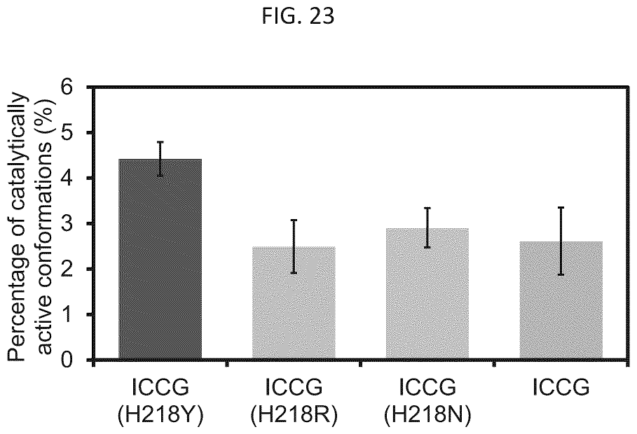

To gain further insights into the impact of H218 mutations on enzyme-PET interactions, we performed MD simulations on ICCG (H218R), which exhibited worse PET depolymerization than ICCG experimentally, and ICCG (H218N), which exhibited better PET depolymerization than ICCG experimentally. For each variant, we performed 10 unbiased MD simulations of 800 ns, and we quantified the percentage of catalytically active conformations for all four variants (FIG. 23). Consistent with the experimental results, ICCG (H218R) exhibited the lowest percentage of catalytically relevant frames (˜2.5%), while ICCG (H218N) exhibited a slightly higher percentage of catalytically relevant frames (˜2.9%) than ICCG (˜2.6%), though still not as high as ICCG (H218Y).

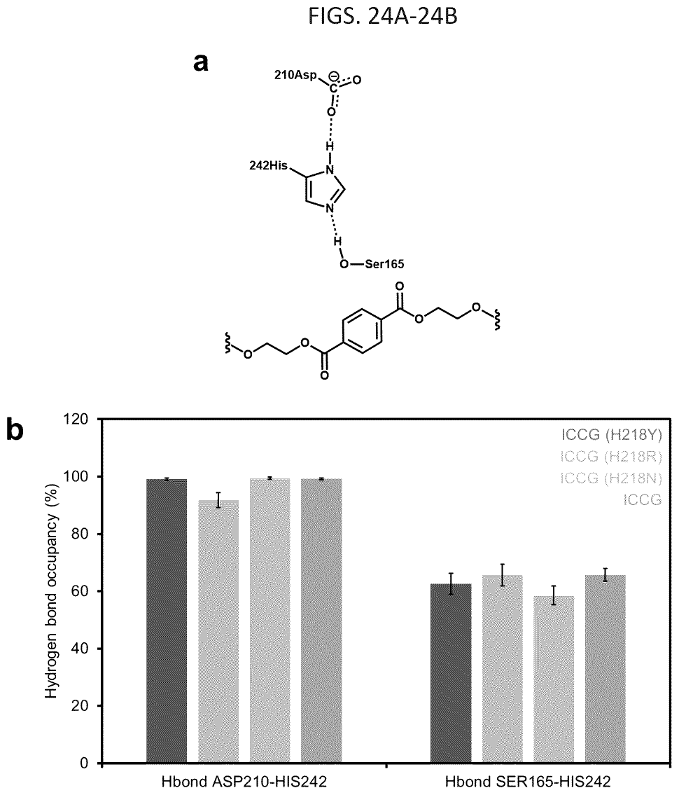

To understand how these two mutations (H218R and H218N) impacted ICCG-substrate interactions, we analyzed RMSF for all mutants along the entire polypeptide chain, hydrogen bonding occupancies for the catalytic triad residues, and the distance between the 218 residue and PET carbonyl oxygens (FIG. 20 and FIG. 21). Strikingly, ICCG (H218R) exhibited much higher RMSF values at several active site residues, suggesting that this mutation destabilizes the active site. Furthermore, the H218R mutation destabilized the catalytic triad; in some MD conformations, Arg218 swung over to the active site to interact with Asp210, preventing it from hydrogen bonding with His242 (FIG. 24). These observations are consistent with the experimental result that LCC (H218R) failed to degrade PET films at 70° C., even though WT LCC showed substantial PET degradation under identical conditions (FIG. 3B). Combining the H218R with the ICCG mutations resulted in partial PET film degradation, suggesting that these thermostability-boosting mutations compensated for the destabilizing effect of the H218R mutation.

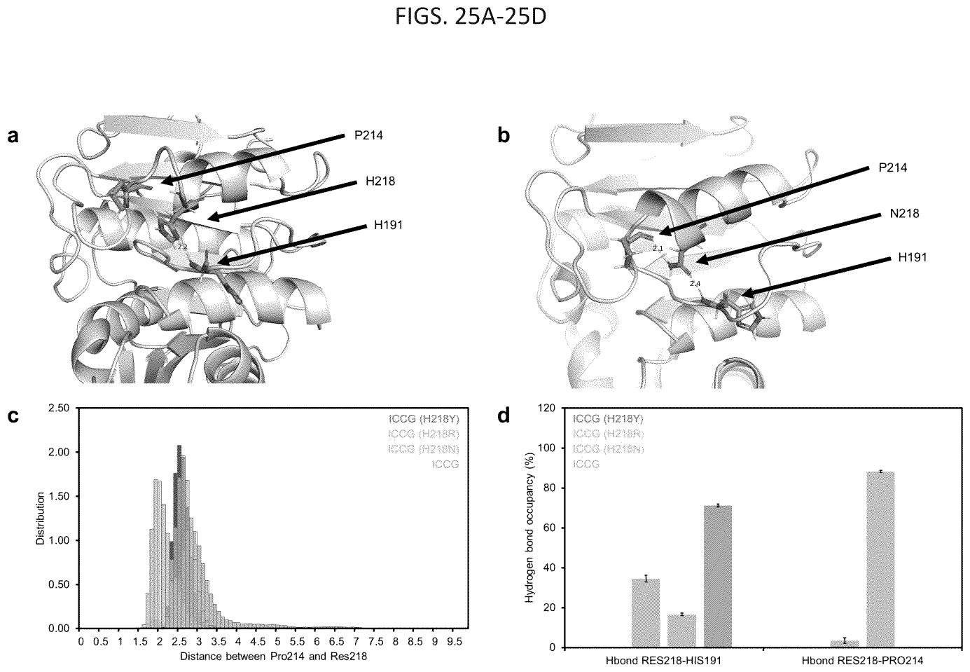

It is less clear why the H218N mutation slightly increased the percentage of catalytically active conformations relative to ICCG. The catalytic triad hydrogen bond occupancies and RMSF values across the entire amino acid sequence are very similar for ICCG (H218N) and ICCG (FIG. 19 and FIG. 20). H218N also did not show a higher propensity to interact with PET carbonyl oxygens (FIG. 21). One difference evident in the ICCG (H218N) simulations is that the Asn218 residue can form simultaneous hydrogen bonds with the backbone carbonyl of P214 and the backbone amide N—H of H191 (FIG. 25). In the ICCG simulations, H218 with the HIE protonation state (where only the ε-nitrogen is protonated) can hydrogen bond with the backbone amide N—H of H191, but not with P214. A simple computational model with H218 in the HID protonation state (where only the δ-nitrogen is protonated) suggests that it could potentially hydrogen bond with carbonyl P214, but not simultaneously with H191. Thus, the simultaneous hydrogen bonding mode of residue 218 with two backbone residues was observed only in ICCG (H218N). Although these hydrogen bonds evidently did not impact the flexibility of any residues in ICCG (H218N), based on RMSF values, it is possible that subtle effects on the positioning of nearby residues (such as Trp190) could have influenced the percentage of catalytically active frames.

### SUMMARY

We have developed a yeast display evolution platform that allows the rapid evaluation of >10⁷ enzyme variants for polymer cleavage activity, increasing the screening throughput by three orders of magnitude relative to existing approaches. More broadly, this work represents an ultrahigh-throughput display platform to select for enzymes that catalyze a bond-breaking reaction on a synthetic substrate. In this Example, we demonstrated the utility of the platform by evaluating more than 27 million PET-hydrolase enzyme variants, leading to the identification of mutations that boost activity on PET plastic. The H218Y mutation improved kinetics of depolymerizing PET plastic in LCC as well as the thermostable variant ICCG. Our yeast display evolution campaign enriched mutations of H218 in LCC, underscoring the importance of residue 218 for PET-depolymerization activity, and our biochemical studies and MD simulations suggest that the H218Y mutation improves enzyme binding to the PET substrate.

Interestingly, the H218N mutation selected using our platform—which substantially improved the PET depolymerization of ICCG—was not identified using the computational method.

Yeast display evolution offers a complementary approach to plate-based screening for PET plastic degradation; while plate-based screens are 1000-fold lower in their throughput, they offer the advantage of directly measuring enzymatic depolymerization of solid PET substrates, rather than PET-resembling probes. Future improvements to this yeast display platform could be realized using synthetic probes containing multiple aromatic esters to better mimic the PET polymer. A drawback of our platform in its current form is that the yeast display selection is performed at room temperature, making it possible to select for mutants that lack the thermostability needed for PET depolymerization at 70° C. Indeed, LCC (H218R) and LCC (H218L) are representative examples of this phenomenon. Nonetheless, our yeast display selection also converged upon LCC (H218Y), which outperformed the parent LCC enzyme in PET degradation at 70° C. The selection also identified the H218N mutation, which slightly boosted the PET film degradation performance of LCC and substantially boosted the performance of the more thermostable ICCG. A promising future direction will be to modify the yeast display procedure to incorporate pressure for the selection of thermostable mutants.

This modular chemogenetic platform for directed evolution is extendable to diverse polymer chains as targets for enzymatic degradation, as the probe is assembled on the yeast surface in two biocompatible steps. The fact that probe cleavage occurs extracellularly means that the platform is not limited to physiological reaction conditions, opening future opportunities to select for catalysts that exhibit high activity under conditions relevant for target processes.

### REFERENCES

(1) Tournier, V; Duquesne, S.; Guillamot, F.; Cramail, H.; Taton, D.; Marty, A.; André, I. Enzymes' Power for Plastics Degradation. Chem. Rev. 2023. https://doi.org/10.1021/acs.chemrev.2c00644.
(2) Ronkvist, Å. M.; Xie, W.; Lu, W.; Gross, R. A. Cutinase-Catalyzed Hydrolysis of Poly(Ethylene Terephthalate). Macromolecules 2009, 42 (14), 5128-5138. https://doi.org/10.1021/ma9005318.
(3) Müller, R.-J.; Schrader, H.; Profe, J.; Dresler, K.; Deckwer, W.-D. Enzymatic Degradation of Poly(Ethylene Terephthalate): Rapid Hydrolyse Using a Hydrolase from T. Fusca. Macromolecular Rapid Communications 2005, 26 (17), 1400-1405. https://doi.org/10.1002/marc.200500410.
(4) Sulaiman, S.; Yamato, S.; Kanaya, E.; Kim, J.-J.; Koga, Y; Takano, K.; Kanaya, S. Isolation of a Novel Cutinase Homolog with Polyethylene Terephthalate-Degrading Activity from Leaf-Branch Compost by Using a Metagenomic Approach. Applied and Environmental Microbiology 2012, 78 (5), 1556-1562. https://doi.org/10.1128/AEM.06725-11.
(5) Thomsen, T. B.; Hunt, C. J.; Meyer, A. S. Influence of Substrate Crystallinity and Glass Transition Temperature on Enzymatic Degradation of Polyethylene Terephthalate (PET). New Biotechnology 2022, 69, 28-35. https://doi.org/10.1016/j.nbt.2022.02.006.
(6) Marten, E.; Müller, R.-J.; Deckwer, W.-D. Studies on the Enzymatic Hydrolysis of Polyesters. II. Aliphatic-Aromatic Copolyesters. Polymer Degradation and Stability 2005, 88 (3), 371-381. https://doi.org/10.1016/j.polymdegradstab.2004.12.001.
(7) Yoshida, S.; Hiraga, K.; Takehana, T.; Taniguchi, I.; Yamaji, H.; Maeda, Y; Toyohara, K.; Miyamoto, K.; Kimura, Y; Oda, K. A Bacterium That Degrades and Assimilates Poly(Ethylene Terephthalate). Science 2016, 351 (6278), 1196-1199. https://doi.org/10.1126/science.aad6359.
(8) Tournier, V; Topham, C. M.; Gilles, A.; David, B.; Folgoas, C.; Moya-Leclair, E.; Kamionka, E.; Desrousseaux, M.-L.; Texier, H.; Gavalda, S.; Cot, M.; Guémard, E.; Dalibey, M.; Nomme, J.; Cioci, G.; Barbe, S.; Chateau, M.; André, I.; Duquesne, S.; Marty, A. An Engineered PET Depolymerase to Break down and Recycle Plastic Bottles. Nature 2020, 580 (7802), 216-219. https://doi.org/10.1038/s41586-020-2149-4.
(9) Zeng, W.; Li, X.; Yang, Y; Min, J.; Huang, J.-W.; Liu, W.; Niu, D.; Yang, X.; Han, X.; Zhang, L.; Dai, L.; Chen, C.-C.; Guo, R.-T. Substrate-Binding Mode of a Thermophilic PET Hydrolase and Engineering the Enzyme to Enhance the Hydrolytic Efficacy. ACS Catal. 2022, 12 (5), 3033-3040. https://doi.org/10.1021/acscatal.1c05800.
(10) Ding, Z.; Xu, G.; Miao, R.; Wu, N.; Zhang, W.; Yao, B.; Guan, F.; Huang, H.; Tian, J. Rational Redesign of Thermophilic PET Hydrolase LCCICCG to Enhance Hydrolysis of High Crystallinity Polyethylene Terephthalates. Journal of Hazardous Materials 2023, 453, 131386. https://doi.org/10.1016/j.jhazmat.2023.131386.
(11) Pirillo, V.; Orlando, M.; Battaglia, C.; Pollegioni, L.; Molla, G. Efficient Polyethylene Terephthalate Degradation at Moderate Temperature: A Protein Engineering Study of LC-Cutinase Highlights the Key Role of Residue 243. The FEBS Journal 2023, 290 (12), 3185-3202. https://doi.org/10.1111/febs.16736.
(12) Lu, H.; Diaz, D. J.; Czarnecki, N. J.; Zhu, C.; Kim, W.; Shroff, R.; Acosta, D. J.; Alexander, B. R.; Cole, H. O.; Zhang, Y; Lynd, N. A.; Ellington, A. D.; Alper, H. S. Machine Learning-Aided Engineering of Hydrolases for PET Depolymerization. Nature 2022, 604 (7907), 662-667. https://doi.org/10.1038/s41586-022-04599-z.
(13) Bell, E. L.; Smithson, R.; Kilbride, S.; Foster, J.; Hardy, F. J.; Ramachandran, S.; Tedstone, A. A.; Haigh, S. J.; Garforth, A. A.; Day, P. J. R.; Levy, C.; Shaver, M. P.; Green, A. P. Directed Evolution of an Efficient and Thermostable PET Depolymerase. Nat Catal 2022, 5 (8), 673-681. https://doi.org/10.1038/s41929-022-00821-3.
(14) Cui, Y; Chen, Y; Liu, X.; Dong, S.; Tian, Y; Qiao, Y; Mitra, R.; Han, J.; Li, C.; Han, X.; Liu, W.; Chen, Q.; Wei, W.; Wang, X.; Du, W.; Tang, S.; Xiang, H.; Liu, H.; Liang, Y; Houk, K. N.; Wu, B. Computational Redesign of a PETase for Plastic Biodegradation under Ambient Condition by the GRAPE Strategy. ACS Catal. 2021, 11 (3), 1340-1350. https://doi.org/10.1021/acscatal.0c05126.
(15) Shi, L.; Liu, P.; Tan, Z.; Zhao, W.; Gao, J.; Gu, Q.; Ma, H.; Liu, H.; Zhu, L. Complete Depolymerization of PET Wastes by an Evolved PET Hydrolase from Directed Evolution. Angewandte Chemie International Edition 2023, 62 (14), e202218390. https://doi.org/10.1002/anie.202218390.
(16) Liu, B.; He, L.; Wang, L.; Li, T.; Li, C.; Liu, H.; Luo, Y; Bao, R. Protein Crystallography and Site-Direct Mutagenesis Analysis of the Poly(Ethylene Terephthalate) Hydrolase PETase from Ideonella sakaiensis. ChemBioChem 2018, 19 (14), 1471-1475. https://doi.org/10.1002/cbic.201800097.
(17) Rennison, A.; Winther, J. R.; Varrone, C. Rational Protein Engineering to Increase the Activity and Stability of IsPETase Using the PROSS Algorithm. Polymers 2021, 13 (22), 3884. https://doi.org/10.3390/polym13223884.
(18) Erickson, E.; Gado, J. E.; Avilán, L.; Bratti, F.; Brizendine, R. K.; Cox, P. A.; Gill, R.; Graham, R.; Kim, D.-J.; König, G.; Michener, W. E.; Poudel, S.; Ramirez, K. J.; Shakespeare, T. J.; Zahn, M.; Boyd, E. S.; Payne, C. M.; DuBois, J. L.; Pickford, A. R.; Beckham, G. T.; McGeehan, J. E. Sourcing Thermotolerant Poly(Ethylene Terephthalate) Hydrolase Scaffolds from Natural Diversity. Nat Commun 2022, 13 (1), 7850. https://doi.org/10.1038/s41467-022-35237-x.
(19) Pfaff, L.; Gao, J.; Li, Z.; Jackering, A.; Weber, G.; Mican, J.; Chen, Y; Dong, W.; Han, X.; Feiler, C. G.; Ao, Y-F.; Badenhorst, C. P. S.; Bednar, D.; Palm, G. J.; Lammers, M.; Damborsky, J.; Strodel, B.; Liu, W.; Bornscheuer, U. T.; Wei, R. Multiple Substrate Binding Mode-Guided Engineering of a Thermophilic PET Hydrolase. ACS Catal. 2022, 12 (15), 9790-9800. https://doi.org/10.1021/acscatal.2c02275.
(20) Hong, H.; Ki, D.; Seo, H.; Park, J.; Jang, J.; Kim, K.-J. Discovery and Rational Engineering of PET Hydrolase with Both Mesophilic and Thermophilic PET Hydrolase Properties. Nature Communications 2023, 14 (1), 4556. https://doi.org/10.1038/s41467-023-40233-w.
(21) Su, T.; Zheng, Y; Li, Q.; Liu, P.; Yuan, Y; Dian, L.; Wang, Q.; Liang, Q.; Qi. Dynamic Docking Assisted Engineering of Hydrolase for Efficient PET Depolymerization. PREPRINT (Version 1) available at Research Square. https://doi.org/10.21203/rs.3.rs-2349308/v1.
(22) Yi, L.; Gebhard, M. C.; Li, Q.; Taft, J. M.; Georgiou, G.; Iverson, B. L. Engineering of TEV Protease Variants by Yeast ER Sequestration Screening (YESS) of Combinatorial Libraries. Proceedings of the National Academy of Sciences 2013, 110 (18), 7229-7234. https://doi.org/10.1073/pnas.1215994110.
(23) Antipov, E.; Cho, A. E.; Wittrup, K. D.; Klibanov, A. M. Highly 1 and d Enantioselective Variants of Horseradish Peroxidase Discovered by an Ultrahigh-Throughput Selection Method. Proceedings of the National Academy of Sciences 2008, 105 (46), 17694-17699. https://doi.org/10.1073/pnas.0809851105.
(24) Lam, S. S.; Martell, J. D.; Kamer, K. J.; Deerinck, T. J.; Ellisman, M. H.; Mootha, V. K.; Ting, A. Y Directed Evolution of APEX2 for Electron Microscopy and Proximity Labeling. Nat Methods 2015, 12 (1), 51-54. https://doi.org/10.1038/nmeth.3179.
(25) Chen, I.; Dorr, B. M.; Liu, D. R. A General Strategy for the Evolution of Bond-Forming Enzymes Using Yeast Display. Proceedings of the National Academy of Sciences 2011, 108 (28), 11399-11404. https://doi.org/10.1073/pnas.1101046108.
(26) Presolski, S. I.; Hong, V.; Cho, S.-H.; Finn, M. G. Tailored Ligand Acceleration of the Cu-Catalyzed Azide-Alkyne Cycloaddition Reaction: Practical and Mechanistic Implications. J. Am. Chem. Soc. 2010, 132 (41), 14570-14576. https://doi.org/10.1021/ja105743g.
(27) Van Deventer, J. A.; Le, D. N.; Zhao, J.; Kehoe, H. P.; Kelly, R. L. A Platform for Constructing, Evaluating, and Screening Bioconjugates on the Yeast Surface. Protein Engineering, Design and Selection 2016, 29 (11), 485-494. https://doi.org/i0.1093/protein/gzw029.
(28) Sulaiman, S.; You, D.-J.; Kanaya, E.; Koga, Y; Kanaya, S. Crystal Structure and Thermodynamic and Kinetic Stability of Metagenome-Derived LC-Cutinase. Biochemistry 2014, 53 (11), 1858-1869. https://doi.org/10.1021/bi401561p.
(29) Boder, E. T.; Wittrup, K. D. Yeast Surface Display for Screening Combinatorial Polypeptide Libraries. Nat Biotechnol 1997, 15 (6), 553-557. https://doi.org/10.1038/nbt0697-553.
(30) Shirke, A. N.; White, C.; Englaender, J. A.; Zwarycz, A.; Butterfoss, G. L.; Linhardt, R. J.; Gross, R. A. Stabilizing Leaf and Branch Compost Cutinase (LCC) with Glycosylation: Mechanism and Effect on PET Hydrolysis. Biochemistry 2018, 57 (7), 1190-1200. https://doi.org/10.1021/acs.biochem.7b01189.
(31) Crnjar, A.; Griñen, A.; Kamerlin, S. C. L.; Ramirez-Sarmiento, C. A. Conformational Selection of a Tryptophan Side Chain Drives the Generalized Increase in Activity of PET Hydrolases through a Ser/Ile Double Mutation. ACS Org. Inorg. Au 2023, 3 (2), 109-119. https://doi.org/10.1021/acsorginorgau.2c00054.
(32) Chen, Z.; Wang, Y; Cheng, Y; Wang, X.; Tong, S.; Yang, H.; Wang, Z. Efficient Biodegradation of Highly Crystallized Polyethylene Terephthalate through Cell Surface Display of Bacterial PETase. Science of The Total Environment 2020, 709, 136138. https://doi.org/10.1016/j.scitotenv.2019.136138.
(33) Avilan, L.; Lichtenstein, B. R.; König, G.; Zahn, M.; Allen, M. D.; Oliveira, L.; Clark, M.; Bemmer, V.; Graham, R.; Austin, H. P.; Dominick, G.; Johnson, C. W.; Beckham, G. T.; McGeehan, J. E.; Pickford, A. R. Concentration-Dependent Inhibition of Mesophilic PETases on Poly(Ethylene Terephthalate) Can Be Eliminated by Enzyme Engineering. ChemSusChem 2023, 16 (8), e202202277. https://doi.org/10.1002/cssc.202202277.
(34) Brizendine, R. K.; Erickson, E.; Haugen, S. J.; Ramirez, K. J.; Miscall, J.; Salvachúa, D.; Pickford, A. R.; Sobkowicz, M. J.; McGeehan, J. E.; Beckham, G. T. Particle Size Reduction of Poly(Ethylene Terephthalate) Increases the Rate of Enzymatic Depolymerization But Does Not Increase the Overall Conversion Extent. ACS Sustainable Chem. Eng. 2022, 10 (28), 9131-9140. https://doi.org/10.1021/acssuschemeng.2c01961.
(35) Thomsen, T. B.; Schubert, S.; Hunt, C. J.; Borch, K.; Jensen, K.; Brask, J.; Westh, P.; Meyer, A. S. Rate Response of Poly(Ethylene Terephthalate)-Hydrolases to Substrate Crystallinity: Basis for Understanding the Lag Phase. ChemSusChem 2023, 16 (13), e202300291. https://doi.org/10.1002/cssc.202300291.
(36) Erickson, E.; Shakespeare, T. J.; Bratti, F.; Buss, B. L.; Graham, R.; Hawkins, M. A.; König, G.; Michener, W. E.; Miscall, J.; Ramirez, K. J.; Rorrer, N. A.; Zahn, M.; Pickford, A. R.; McGeehan, J. E.; Beckham, G. T. Comparative Performance of PETase as a Function of Reaction Conditions, Substrate Properties, and Product Accumulation. ChemSusChem 2022, 15 (1), e202101932. https://doi.org/10.1002/cssc.202101932.
(37) Bååth, J. A.; Borch, K.; Jensen, K.; Brask, J.; Westh, P. Comparative Biochemistry of Four Polyester (PET) Hydrolases**. ChemBioChem 2021, 22 (9), 1627-1637. https://doi.org/10.1002/cbic.202000793.
(38) Kaabel, S.; Therien, J. P. D.; Deschênes, C. E.; Duncan, D.; Friščić, T.; Auclair, K. Enzymatic Depolymerization of Highly Crystalline Polyethylene Terephthalate Enabled in Moist-Solid Reaction Mixtures. Proceedings of the National Academy of Sciences 2021, 118 (29), e2026452118. https://doi.org/10.1073/pnas.2026452118.
(39) Fecker, T.; Galaz-Davison, P.; Engelberger, F.; Narui, Y; Sotomayor, M.; Parra, L. P.; Ramirez-Sarmiento, C. A. Active Site Flexibility as a Hallmark for Efficient PET Degradation by I. sakaiensis PETase. Biophysical Journal 2018, 114 (6), 1302-1312. https://doi.org/10.1016/j.bpj.2018.02.005.
(40) Chen, C.-C.; Han, X.; Li, X.; Jiang, P.; Niu, D.; Ma, L.; Liu, W.; Li, S.; Qu, Y; Hu, H.; Min, J.; Yang, Y; Zhang, L.; Zeng, W.; Huang, J.-W.; Dai, L.; Guo, R.-T. General Features to Enhance Enzymatic Activity of Poly(Ethylene Terephthalate) Hydrolysis. Nat Catal 2021, 4 (5), 425-430. https://doi.org/10.1038/s41929-021-00616-y.
(41) McGaughey, G. B.; Gagné, M.; Rappé, A. K. π-Stacking Interactions: ALIVE AND WELL IN PROTEINS *. Journal of Biological Chemistry 1998, 273 (25), 15458-15463. https://doi.org/10.1074/jbc.273.25.15458.

### General Information

### Materials

All chemicals, solvents and other reagents were obtained from commercial suppliers (Sigma Aldrich, ThermoFisher, TCI, Santa Cruz, NEB, DOT Scientific, US Biological) and used as received unless otherwise indicated. Phosphate buffered saline (PBS) was prepared as follows: 137 mM NaCl, 2.7 mM KCl, 10 mM Na₂HPO₄, 1.8 mM KH₂PO₄ in ultrapure water. Where indicated, the pH was adjusted to 5.5 via addition of HCl. For cell washing steps of yeast labeling experiments, 1 mg/mL bovine serum albumin (BSA) was added to PBS, referred to as PBS-B. LCC purification buffer was prepared as follows: 25 mM Tris, 200 mM NaCl, with pH was adjusted to 7.5 via addition of HCl.¹

Polyethylene terephthalate (PET) was acquired in two forms from Goodfellow Cambridge Ltd.: amorphous film (0.25 mm thickness, product code ES30-FM-000145, “Batch 1” lot number LS520621, “Batch 2” lot number LS584191), and semicrystalline powder (300 μm maximum particle size, product code ES30-PD-000132). “Batch 2” film was used for all main text FIGS. 1-5, other than FIG. 4b where “Batch 1” was used. PET film batch used in supporting experiments is indicated in the figure description.

S. cerevisiae strain BJ5465 was obtained from ATCC (product no. 208289, batch no. 70003929). E. coli strain DH5α was obtained from ThermoFisher Scientific (catalog no. 12034013, lot no. 2014131). E. coli strain C41(DE3) was obtained from LGC (catalog no. 60442-1, lot no. 18357)

Yeast culture media (YPD or SDCAA) and yeast induction media (SGCAA) were prepared following a published procedure.²

Rotary evaporation was performed using a Buchi R-300 Rotovap.

### HPLC Analysis

Reverse-phase HPLC analysis was performed on a Shimadzu Prominence HPLC system with detection based on absorbance at 254 nm. Small molecule analysis was performed using a 4.6×50 mm XBridge C18 (3.5 μm) column (with or without at Shim-Pack GIST C18 guard column (5 μm, 4.0×10 mm)) using a 1.0 mL/min flow rate using ultrapure water with 0.1% trifluoroacetic acid as mobile phase A and acetonitrile as mobile phase B (with one of the following gradients: 1: no guard column, 10-40% B over 3.5 minutes, then 40-90% B over 0.5 minutes, or 2: with guard column, 10-25% B over 0.5 minutes, then 25-40% B over 3.5 minutes, then 40-90% B over 0.5 minutes. Both gradients were followed by a 1-minute wash at 90% B and then re-equilibration at 10% B), with a column oven temperature of 30° C. Samples of small molecule and PET film reactions were prepared for HPLC analysis by diluting the reaction 1:10 or 1:20 (v/v) in MeOH. Sample injection volume was 5 μL.

### Preparation of HPLC Calibration Curves

Terephthalic acid (TPA), mono(2-hydroxyethyl) terephthalic acid (MHET), and bis(2-hydroxyethyl terephthalate (BHET) were obtained commercially in order to prepare standard solutions for HPLC calibration curves. A 100 mM solution of each compound in DMSO was prepared, then sequentially diluted in 500 mM HEPES buffer pH 8 to create a concentration series of 50 to 0.78 mM. The dilution series was prepared in duplicate from individual stocks. Each solution was then analyzed by HPLC as described above. A calibration curve was prepared by taking the average peak area at each concentration plotted against the concentration value. Standard linear-regression fitting was applied with the y-intercept set to 0.

### NMR Spectroscopy

¹H-NMR spectra were recorded on a Bruker Avance 500 MHz spectrometer. Chemical shifts (δ) are given in parts per million and referenced to TMS.

¹³C{¹H}-NMR spectra were recorded on a Bruker Avance 500 MHz spectrometer. Chemical shifts (δ) are given in parts per million and referenced to TMS.

### Mass Spectrometry

ESI-MS small molecule high resolution mass spectrometry data were obtained on a Thermo Q Exactive™ Plus by the mass spectrometry facility at the University of Wisconsin-Madison. The purchase of the Thermo Q Exactive™ Plus in 2015 was funded by NIH Award 1S10 OD0200221 to the Department of Chemistry.

### Fluorescence Activated Cell Sorting

Flow cytometry analysis was performed using an Attune NxT V6 Flow Cytometer (ThermoFisher Scientific) equipped with 561 and 633 nm lasers and appropriate emission filters (585/16 for phycoerythrin, and 670/14 for AlexaFluor647). Cell sorting was performed using a FACSAria Cell Sorter (BD Biosciences) with 561 and 633 nm lasers and corresponding filters (582/15 for phycoerythrin, 660/20 for AlexaFluor647).

### Differential Scanning Calorimetry (DSC) of PET Films

PET sample crystallinity was determined using a DSC 250 (TA Instruments). PET samples (2-10 mg) were placed in aluminum Tzero pans with a Tzero sample lid. Samples were heated from 20 to 290° C. at a rate of 10° C./min. The percent crystallinity was determined by comparing the measured melting enthalpy to the reference value for 100% crystalline PET (140 J/g) according to the following equation¹:

$${\%}_{crystallini} = \frac{( Δ {H}_{m} - Δ {H}_{cc} )}{{140}_{g}^{L}} * 100$$

Where ΔHₘ is the measured melting enthalpy and ΔH_{cc} is the measured enthalpy of cold crystallization. T_{g} and normalized enthalpy values were calculated using TA Instruments TRIOS software.

### Experimental Procedures

### BHET Hydrolysis Assay

BHET reactions were performed at 70° C. with 1 mM final concentration BHET and 50 nM final concentration enzyme in 500 mM HEPES pH 8.0.³ 940 μL of HEPES buffer was added to a 1.5 mL polypropylene tube followed by the addition of 50 μL of 1 μM enzyme in PBS pH 7.4. Enzyme solutions were placed in a 70° C. heat block for 30 seconds before initiating the reaction by adding 10 μL of 100 mM BHET in DMSO. Reactions were vortexed briefly to mix then placed back in the heat block. Timing was initiated immediately upon vortexing, and 20 μL aliquots were withdrawn after 5, 10, 15, and 30 minutes and diluted into 180 μL of methanol. The samples were analyzed via HPLC using the method described above.

For BHET hydrolysis using enzyme expressed on yeast cell surface: 30 million yeast (based on OD₆₀₀ measurement on a ThermoFisher Scientific Nanodrop One) containing pCTCON2-Aga2P-LCC (expression of which was either induced or uninduced; see yeast display protocols below) were washed twice with PBS-B. For each wash, the yeast cell suspension was spun down at 13,000×g for 1 min, the supernatant was removed, and the pellet was suspended in 200 μL of PBS-B. After the two washes, yeast pellets were suspended in 200 μL of 1 mM BHET in PBS pH 7.4 in a 1.5 mL Eppendorf tube. The samples were vortexed thoroughly and incubated in a thermomixer (Eppendorf) set to 24° C. with 800 rpm shaking. Time points were taken by pelleting the yeast and removing 10 μL of supernatant to be combined with 90 μL of methanol for HPLC analysis (see section above). After the time point was taken, the yeast were suspended and returned to the thermomixer. Time points were taken at 30, 60, 120, and 1440 minutes.

### PET Hydrolysis Reactions

Standard PET film digests were performed with 6 mm (˜8 mg) PET film disks (Goodfellow, untreated) and 500 nM enzyme in 500 mM HEPES buffer pH 8 at 1 mL total volume. For inverse Michaelis-Menten experiment, constant PET film loading with various enzyme concentrations were used, as indicated in corresponding figure. Samples were vortexed thoroughly and incubated stationary at 70° C. for the indicated durations. Reactions were carried out at 1 mL total volume in 1.5 mL polypropylene tubes. Reactions were sampled and analyzed as described in the HPLC section.

For pseudo Michaelis-Menten experiment (FIG. 4b), PET film strips were weighed into 1.5 mL Eppendorf tubes with four replicates of each target mass. The actual masses of each replicate were averaged to give the masses seen on the x-axis. The actual masses were within 1.1 mg of the averaged mass. Two replicates were performed for each enzyme, ICCG and ICCG (H218Y) at each target mass. Initial rates were measured by total TPA, MHET, and BHET concentration measured after 2, 3, and 4 hours of incubation with 100 nM enzyme at a 1 mL volume in HEPES buffer, pH 8 and analyzed as described in the HPLC methods section.

GraphPad Prism was used to analyze the pseudo Michaelis-Menten data using the “Enzyme kinetics—Michaelis-Menten” function. Nonlinear regression was done to determine the Michaelis-Menten fit, shown in dashed lines in FIG. 4b.

Semicrystalline PET powder experiments (FIG. 4c) were performed with 8-10 mg of semicrystalline PET powder (Goodfellow, untreated) and 1 μM enzyme in 500 mM HEPES buffer pH 8 at 1 mL total volume. Samples were incubated in a thermomixer at 70° C., 1500 rpm. Reactions were carried out in 1.5 mL polypropylene tubes. At 30, 60, 120, 180, and 240 minutes, samples were removed from the thermomixer, centrifuged for 1 minute at 13,000×g to pellet the PET powder, and 10 μL of the supernatant was removed for analysis, as described in the HPLC section.

### Yeast Cell Culture

Saccharomyces cerevisiae strain BJ5465 was cultured according to previously published protocols.^{2,4} Cells were propagated at 30° C. in yeast extract peptone dextrose (YPD) complete medium. BJ5465 yeast were first transformed with a YIP plasmid that had been linearized by digestion with the restriction enzyme BsiWI. The YIP plasmid is designed for integration into the yeast genome, and contains the native Aga1P protein sequence.^{5,24} The YIP plasmid was modified as follows: after the Aga1P signal sequence (residue 1-32), an AflII restriction site (LK), followed by the V5 epitope tag (GKPIPNPLLGLDST) (SEQ ID NO: 2), followed by a SacII restriction site (PR), followed by the AP epitope tag (GLNDIFEAQKIEWHE) (SEQ ID NO: 3), followed by an NheI restriction site (AS) was inserted, followed by the rest of the mature Aga1P sequence. Transformation was performed using the Frozen E-Z Yeast Transformation II Kit (Zymo Research) according to the manufacturer's protocols. Transformed yeast were plated on SDCAA media lacking uracil, allowing for selection of individual colonies constitutively expressing Aga1P. Aga1P-expressing yeast were then made competent for transformation using the Frozen E-Z Yeast Transformation II Kit, followed by transformation with the appropriate pCTCON2 plasmid using the same kit. Transformed cells containing the Trp1 gene were selected on synthetic dextrose plus casein amino acid (SDCAA) plates. Yeast cell culture and induction of pCTCON2 construct expression were performed as described previously.⁶

### Generation of Error-Prone Libraries for Yeast Display

Libraries of LCC mutants were generated using error-prone PCR (epPCR), following previously published protocols.^{2,4,6} 830 ng of pCTCON2 vector containing the wild-type LCC gene was used as the template for the first round of epPCR. Amplification proceeded for 10 rounds with 0.4 μM Con.2 forward and reverse primers (F: 5′ CTAGTGGTGGAGGAGGCTCTGGTGGAG GCGGTAGCGGAGGCTGGAGGGTCGGCTAGC (SEQ ID NO: 4), R: 5′-TATCMATCTCGAGCTATTACAAGTCCTCTTCAGAAATAAGCTTTTGTTCGGATCC-3′) (SEQ ID NO: 5),⁶ 2 mM MgCl₂, 5 units of Taq polymerase (NEB), and 2 μM each of the mutagenic nucleotide analogs 8-oxo-2′-deoxyguaniosine-5′-triphosphate (8-oxo-dGTP) and 6H,8H-3,4-dihydro-pyrimido(4,5-c)(1,2)oxazin-7-one-8-β-D.-2-deoxy-ribofuranoside-5-triphosphate (dPTP) (Jena Bioscience). The error-prone LCC gene library was then gel purified (QIAquick Gel Extraction Kit, Qiagen) and re-amplified for another 30 cycles under normal PCR conditions using the HomR forward and reverse primers (F: 5′ CAAGGTCTGCAGGCTAGTTGGTGGAGGAGGCTCTGGTG-3 (SEQ ID NO: 6) R: 5′CTACACTGTTGTTIATCAGATCTCGAGCTATTACAAGTC-3′) (SEQ ID NO: 7)⁶ the mutated gene library was combined with linearized pCTCON2 vector backbone (6 μg insert:1 μg vector) and subjected to ethanol precipitation. The precipitated DNA mixture was resuspended in nuclease-free water and electroporated into electrocompetent S. cerevisiae BJ5465 yeast constitutively expressing Aga1P. Electroporation was performed using an Eppendorf Eporator. The electroporated culture was rescued in 125 mL of SDCAA media supplemented with 100 units/mL penicillin, 100 μg/mL streptomycin and 50 μg/mL kanamycin for 2 d at 30° C. Transformation efficiency (i.e, maximum library diversity) was determined to be 27×10⁷ Sanger sequencing of representative clones showed an average of 2.3 mutated nucleotides per clone with a range of 0-4; see Method section below for details on plasmid extraction and sequencing,

### Directed Evolution & CuAAC Cell Labeling Protocol:

Below is a general procedure describing cell labeling for monoclonal wild-type LCC-displaying yeast for analytical experiments, as well as the initial error-prone LCC library and subsequent sorted libraries (rounds 1-3) for sorting experiments. A table at the end provides specific solution volumes used at each step for the different samples. Some procedural details also differed for labeling small, non-library samples. Specifically, a thermomixer at 900 rpm was used for mixing during the labeling steps instead of horizontal shaking on a rotating platform. Also, the labeling was done in a 1.5 mL Eppendorf tube in place of a 15 or 50 mL conical used for the library samples. A standardized number of cells was chosen for the non-library samples, provided in Table 1.

S. cerevisiae cells containing wild-type LCC, the original mutated LCC library, or subsequently sorted libraries were thawed on ice and added to a sterile baffled flask with SDCAA media. The cells were incubated at 30° C. for 48 hours to grow to saturation and then a sufficient volume to maintain ten-fold library diversity coverage was diluted 1:9 in SGCAA media to induce surface expression. The culture was allowed to grow for 20 hours to saturation. OD₆₀₀ was measured to determine cell density. An aliquot of cells equal to ten-fold over coverage of library was removed and spun down for 2 minutes at 4000×g at 20° C. The supernatant was removed, and the cells washed twice with PBS-B pH 7.4. The cell pellet was resuspended in PBS pH 7.4 containing 100 mM NaHCO₃ and 0.665 mM NHS-PEG₈-Azide (Quanta Biodesign). The pellet was mixed via inversion and placed horizontally on a rotating platform for 30 minutes at room temperature. The solution was also manually mixed by inversion after 10 and 20 minutes.

After the NHS-azide labeling, the sample was removed from rotation and spun down for 2 minutes at 4,000×g at 20° C. The supernatant solution was removed, and the cells washed twice with PBS-B pH 7.4. Separately, a solution containing 1:100 μL anti-c-myc antibody (EMD Millipore Co., AB3253) in PBS pH 7.4 was prepared. The pellet following the second wash was resuspended in the anti-c-myc antibody solution. The tube was placed on ice and set horizontally on a rotating platform to label for 30 minutes with manual inversion at 10 minutes and 20 minutes.

After 30 minutes, the sample was removed from rotation and ice and spun down for 2 minutes at 4,000×g at 20° C. The supernatant solution was removed, and the cells were washed twice with PBS-B pH 7.4. Separately, a solution containing 1:100 AlexaFluor™ 647 Goat anti-chicken IgY (H+L) (Invitrogen, A21449) in PBS pH 7.4 was prepared and added to the washed pellet. The pellet was resuspended and placed on ice on a shaking platform for 30 minutes to label.

After 30 minutes, the sample was removed from rotation and spun down for 2 minutes at 4,000×g at 20° C. The supernatant solution was removed, and the cells washed twice with PBS-B pH 7.4. The pellet following the second wash was resuspended in PBS pH 5.5 (adjusted via addition of HCl), containing 5 mM sodium ascorbate in ultrapure water. A premade solution of Cu:THPTA 1:5 (prepared by combining 150 μL of 20 mM CuSO₄ in ultrapure water and 300 μL of 50 mM tris-hydroxypropyltriazolylmethylamine (THPTA) in DMSO) was added to reach 0.150 Cu and 0.750 mM THPTA.^{7,8} This was followed by addition of probe 3 (see chemical structure below) in DMSO to reach 0.01 mM. The solution was mixed via inversion and placed horizontally on a rotating platform for 1 hour at room temperature with rotation sufficient to prevent the cells from settling to the bottom of the tube.

After 1 hour, the sample was removed from rotation and spun down for 2 minutes at 4,000×g at 20° C. The supernatant solution was removed, and the cells were washed twice with PBS-B pH 5.5. A fluorescent labeling solution was prepared containing 1:100 streptavidin-phycoerythrin (sAv-PE, Invitrogen, S866) in PBS pH 5.5. The sAv-PE solution was transferred to the cell pellet and vortexed briefly to resuspend. The solution was placed horizontally on ice on a rotating platform for 30 minutes with rotation sufficient to prevent the cells from settling to the bottom of the tube.

After 30 minutes, the solution was removed from rotation and ice and spun down for 2 minutes at 4,000×g at 20° C. The supernatant of the solution was removed, and the cells washed twice with PBS-B pH 5.5. The cells were then resuspended in PBS-B pH 5.5 to a density of approximately 12 million cell/mL and then passed through a 35 μm sterile filter. The solution was stored at 4° C. until it was analyzed on the FACS instrumentation. Gates were drawn to collect cells with positive AF647 signal that also had low PE signal. Sorted cells were collected into a 5 mL solution of 2.5 mL SDCAA media and 2.5 mL PBS-B pH 7.4 containing 1% penicillin-streptomycin, 50 μg/mL kanamycin and 10 μg/mL tetracyline. The cells were allowed to grow to saturation in SDCAA for 48 hours before being passaged once between rounds of sorting.

Table of solution volumes:

| Sample: | General | Rd 1 Sort | Rd 2 Sort | Rd 3 Sort |
|---|---|---|---|---|
| Approximate Number of Cells Labeled | 3,000,000 | 300,000,000 | 48,000,000 | 24,000,000 |
| PBS-B washes (mL) | 0.2 | 15 | 3 | 1.5 |
| NHS-PEG₈-azide labeling solution (mL) | 0.2 | 15 | 3 | 1.5 |
| Antibody labeling solutions (mL) | 0.05 | 3.5 | 0.625 | 0.313 |
| CuAAC click labeling solution (mL) | 0.2 | 15 | 3 | 1.5 |
| sAv-PE labeling solution (mL) | 0.05 | 3.5 | 0.625 | 0.313 |
| Final resuspension volume (mL) | 1 | 25 | 4 | 2 |

### Sequencing of Sorted Libraries

S. cerevisiae cells following the third sort were grown in 5 mL of SDCAA media to saturation before four 500 μL aliquots were removed and separately added to four 15 mL culture tubes containing 4.5 mL SDCAA media. After overnight growth the saturated yeast cultures were centrifuged for 2 minutes at 4000×g at room temperature, and the supernatant was discarded. Plasmids were extracted from the yeast using the Zymoprep Yeast Plasmid Miniprep I kit (Zymo Research) according to the manufacturer's instructions. The recovered plasmids were transformed into chemically competent DH5α E. coli. Monoclonal E. coli colonies from each plate were grown to saturation in 5 mL LB/amp media, then miniprepped using the Omega Bio-tek plasmid DNA purification kit (Omega Bio-tek Inc.) according to the manufacturer's instructions. Purified plasmids were submitted for Sanger sequencing. Sequencing data were analyzed using the program MUTATO.⁹

### Protein Purification

A C41 DE3 colony was inoculated from a monoclonal LB/amp plate into 5 mL of LB/amp and incubated at 37° C. overnight with shaking at 220 rpm. The following day after the culture had grown to saturation, 1% of the volume of the cell suspension was transferred into TB/amp media—typically 0.5 mL into 50 mL or all 5 mL into 500 mL—into a baffled flask that was sufficiently large—typically 125 or 1000 mL—to provide sufficient head space to fully oxygenate the media. The culture was grown in an incubator at 37° C. with shaking at 220 rpm until it reached an OD of 0.6-0.8, at which time 1000×IPTG was added to a final concentration of 1 mM, and the flask was transferred to an incubator set to 18° C. with shaking at 200 rpm. The culture was grown for 16-24 hours. After the expression period, the cells were pelleted via centrifugation for 30 minutes at 4,000×g at 4° C. The supernatant was discarded, and the cell pellet was resuspended in lysis buffer equivalent to 4 mL total buffer/gram of cells. The LCC lysis buffer is composed of 25 mM Tris 200 mM NaCl pH adjusted to 7.5 with HCl, 1 mg/mL lysozyme, 1 μL/mL 1 M MgCl₂, and 0.6 μL/mL DNAse I. The pellet and lysis solution were vigorously vortexed to resuspend until homogeneity was achieved. The solution was placed on ice on an orbital shaker to lyse for 45 minutes at a high rate of rotation with vortexing every 15 minutes to ensure the pellet remained completely suspended. The solution was then subjected to sonication with a ¼″ probe at 30% amplitude for 10 min of on time (1 second on, 1 second off). Following sonication, the lysate was spun down to pellet the cell debris for 30 minutes at 4° C. at 41,300×g in a fixed angle rotor.

In a protein purification column, 1-3 mL of Ni-NTA beads (GoldBio) were washed with ˜5 mL of LCC purification buffer before the clarified lysate supernatant was transferred to the washed beads. The column with the beads and clarified lysate supernatant were placed on ice for 30 minutes on an orbital shaker with moderate shaking intensity. After the nutation period, clarified lysate supernatant was run through the column. The beads were then washed with at least 10 mL of 60 mM imidazole LCC purification buffer before the protein was eluted with ˜6 mL of 300 mM imidazole LCC purification buffer. The elution buffer was exchanged into PBS pH 7.4 or LCC purification buffer by either Amicon (Millipore Sigma) filtration/concentration (3 washes of ˜20 mL of PBS pH 7.4) or dialysis in 3.5 k MW SnakeSkin Dialysis Tubing (ThermoFischer Scientific). For dialysis, the protein solution was transferred to a dialysis tubing pouch and placed into 1 L of desired buffer for 1 hour then transferred to 1 L of fresh buffer for 2 hours before being transferred to an additional fresh 1 L of desired buffer and dialyzed overnight. Generally, enzyme solution was then aliquoted and flash frozen in liquid nitrogen for storage at −80° C. In some enzyme preparations a precipitate of insoluble protein aggregates formed during dialysis. After thawing, all samples were centrifuged to pellet the precipitate, and the soluble supernatant portion was moved to a new epi-tube and used for all reactions and concentration determinations.

Enzyme concentration was determined using the bicinchoninc acid (BCA) assay.¹⁰ Protein purity was determined via 12% SDS-PAGE gels. Protein gels were visualized via Coomassie Brilliant Blue staining and destaining following manufacturer's recommended protocol and imaged with an Azure C400 Gel Imaging System (Azure Biosystems) using the visible setting.

### Synthesis

### Overview of Synthetic Scheme

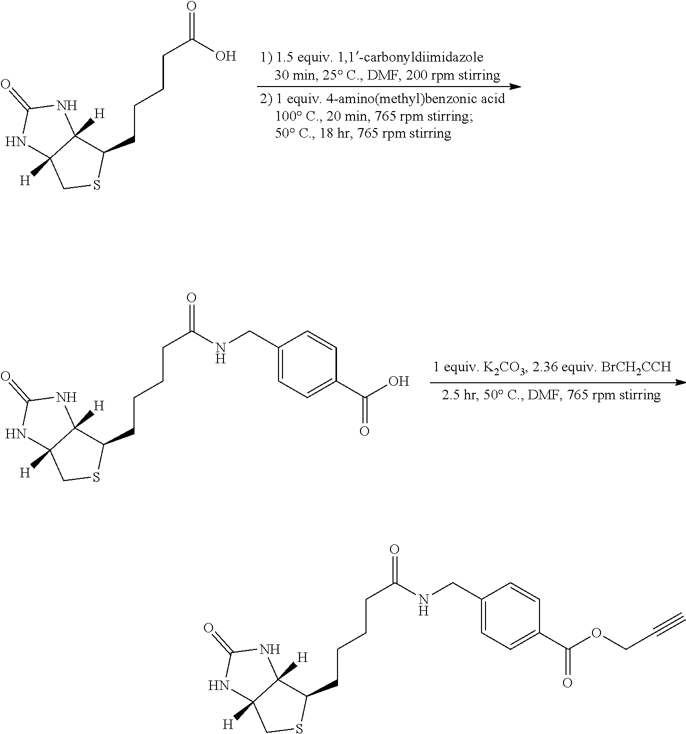 [SMILES: O=C(O)CCCC[C@H]1SC[C@@H]2NC(=O)N[C@@H]21 ; O=C(CCCC[C@H]1SC[C@@H]2NC(=O)N[C@@H]21)NCc1ccc(C(=O)O)cc1 ; C#CCOC(=O)c1ccc(CNC(=O)CCCC[C@H]2SC[C@@H]3NC(=O)N[C@@H]32)cc1]

### Synthesis of Propargyl Amide of Biotin (1)

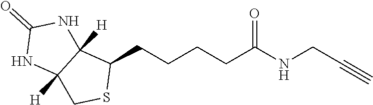 [SMILES: C#CCNC(=O)CCCC[C@H]1SC[C@@H]2NC(=O)N[C@@H]21]

4-((5-((3aS,4S,6aR)-2-oxohexahydro-1H-thieno[3,4-d]imidazol-4-yl)pentanamide (1) was synthesized according to a previously published procedure.¹¹ 200 mg (0.819 mmol) of D-biotin was dissolved in 8 mL 3:1 acetonitrile:methanol, and 62.5 μL (1.2 equiv., 0.982 mmol) of propargyl amine was added dropwise over 1 minute. 204.0 mg (1.3 equiv., 1.064 mmol) EDC-HCl was added over the course of 5 minutes to the solution, stirring at 850 rpm at room temperature. The vial was then sealed with a septum and connected to a nitrogen line to proceed overnight. The solvent was removed via rotary evaporation, and 2 mL methanol was added to the solid. The crude product solution was filtered through diatomaceous earth to remove the EDC-urea byproduct. The filtrate was concentrated via rotary evaporation and added to CH₂Cl₂ causing a precipitate to form. This was filtered once more, concentrated again, and loaded onto a silica gel column run with a constant mobile phase of 10:1 CH₂Cl₂:methanol. Fractions containing the desired product were combined, and the solvent was removed via rotary evaporation to afford 60 mg (26% yield) of 1. ¹H NMR results matched literature reported values.¹¹

### Synthesis of Biotin Aminomethylbenzoic Acid (2)

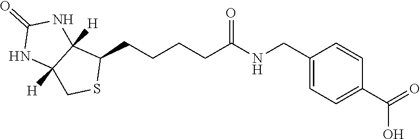 [SMILES: O=C(CCCC[C@H]1SC[C@@H]2NC(=O)N[C@@H]21)NCc1ccc(C(=O)O)cc1]

[4-((5-((3aS,4S,6aR)-2-oxohexahydro-1H-thieno[3,4-d]imidazol-4-yl)pentanamido)methyl)benzoic acid](2) was synthesized according to a previously reported procedure.¹² 202.6 mg (0.829 mmol) of D-biotin was dissolved in 5 mL of dry DMF in a 25 mL glass vial. The solution was heated to 70° C. while stirred at 765 rpm for 15 minutes until the biotin dissolved. The vial was removed from heat and allowed to cool to room temperature. 205.6 mg (1.5 equiv., 1.27 mmol) of 1,1′-carbonyldiimidazole was added and stirred at room temperature for 30 minutes at 200 rpm. Next, 131.6 mg (1 equiv., 0.871 mmol) of 4-amino(methyl)benzoic acid was added, the vial was sealed with a septum and connected to a nitrogen line, heated to 100° C. for 20 minutes, then reduced to 50° C. for 18 hours while stirring at 765 rpm. The reaction was cooled to room temperature, and 6 mL of CH₂Cl₂ was added to yield a white precipitate. The precipitate was isolated via vacuum filtration, washed with 4 mL of CH₂Cl₂, and left on a frit until dry. 246 mg (0.651 mmol, 78.4%) of 2 was afforded with high purity. ¹H NMR results matched literature reported values.¹²

### Synthesis of Propargyl Biotin Aminomethylbenzoate (3)

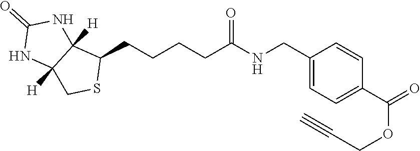 [SMILES: C#CCOC(=O)c1ccc(CNC(=O)CCCC[C@H]2SC[C@@H]3NC(=O)N[C@@H]32)cc1]

prop-2-yn-1-yl 4-((5-((3aS,4S,6aR)-2-oxohexahydro-1H-thieno[3,4-d]imidazol-4-yl)pentanamido)methyl)benzoate] (3). 73.9 mg (0.196 mmol) of 2 was added to a 25 mL vial and dissolved in 4 mL of dry dimethylformamide. 28 mg (1 equiv., 0.202 mmol) of potassium carbonate was added and the solution heated to 50° C. with stirring at 765 rpm. The vial was sealed with a septum and connected to a nitrogen line. 35 μL (2.36 equiv., 0.462 mmol) of propargyl bromide was added dropwise over 45 seconds via a glass syringe. The solution was left to react for 2.5 hours at 50° C. with stirring at 765 rpm. The solution was subsequently removed from the heat and cooled to room temperature. 15 mL of ultrapure water was added to quench the reaction. The mixture was transferred to a 125 mL separatory funnel and the product extracted 3 times with 20 mL of ethyl acetate. The combined extraction was washed with 20 mL of saturated aqueous brine. The organic phase was collected, dried with Na₂SO₄, and decanted into a tared vial. The ethyl acetate was removed via rotary evaporation to yield a beige powder. 78.4 mg were isolated (0.189 mmol, 96.4% yield).

¹H NMR (500 MHz, DMSO) δ 8.41 (t, J=6.0 Hz, 1H), 7.97-7.90 (m, 2H), 7.43-7.37 (m, 2H), 6.41 (t, J=1.8 Hz, 1H), 6.34 (s, 1H), 4.94 (d, J=2.5 Hz, 2H), 4.36-4.27 (m, 3H), 4.12 (ddd, J=7.7, 4.4, 1.9 Hz, 1H), 3.60 (t, J=2.4 Hz, 1H), 3.10 (ddd, J=8.6, 6.1, 4.4 Hz, 1H), 2.83 (dd, J=12.4, 5.1 Hz, 1H), 2.58 (d, J=12.4 Hz, 1H), 2.16 (t, J=7.4 Hz, 2H), 1.68-1.42 (m, 4H), 1.41-1.25 (m, 2H).

¹³C NMR (126 MHz, DMSO) δ 172.24, 164.86, 162.68, 145.97, 129.36, 127.40, 78.45, 77.92, 61.03, 59.19, 55.42, 52.39, 41.75, 35.11, 28.22, 28.02, 25.25.

MS: [(M+H)⁺] calculated: 416.1644, found: 416.1632; [(M+Na)⁺] calculated: 438.1458, found: 438.1452

### Docking of PET₄ to Cutinase

The 3D structure of PET₄ molecule was built using PyMOL 2.5.2. The ICCG structure was obtained from PDB ID: 7VVE,¹³ with mono(2-hydroxyethyl) terephthalic acid removed and residue 165 mutated from alanine to serine. The binding cavity for docking was defined using Autodock 4.2.¹⁴ Finally, docking was performed using Autodock vina.¹⁵

### Assignment of Force Field Parameters for PET₄

To obtain the force field of PET₄, geometry optimization and electrostatic potential calculation were done using Gaussian 16. Geometry optimization was performed using Becke, three-parameter, Lee-Yang-Parr method and basis set 6-31 g* (no. B3LYP/6-31 G*). We then performed electrostatic potential calculation using the Hartree-Fock method with basis set 6-31 g* (no. HF/6-31 g*). The partial charges of PET₄ were then fitted using RESP module in AmberTools21. The non-bonded and bonded parameters of PET₄ were obtained from the GAFF forcefield.¹⁶

Molecular Dynamics (MD) simulations of ICCG or ICCG variant in complex with PET₄ Molecular Dynamics (MD) simulations were performed using Gromacs 2021.5.¹⁷ We adopted the Amber14sb forcefield for proteins.¹⁸ We performed two kinds of MD simulations, a complex of either ICCG or ICCG (H218Y) with PET₄. For the ICCG systems, His218 is in HIE protonation state (neutral and N^{ε} is protonated). For the ICCG (H218Y), ICCG (H218R), and ICCG (H218N) systems, His218 is mutated to tyrosine, arginine, and asparagine, respectively, using PyMOL 2.5.2.¹⁹ The structure from docking is used as the initial structure for MD simulations of both systems. For both systems, the Coulombic interactions were computed using the Particle Mesh Ewald (MWE) algorithm, with the short-range cut off at 1 nm. The Lennard-Jones interactions were also cut off at 1 nm. The initial structure was put in a dodecahedron box such that the distance between the complex and the edge of the box was at least 1 nm. The system was then solvated in TIP3P water,²⁰ and 26 sodium ions and 32 chloride ions were added to neutralize the systems and create a 0.15 mol/L salt concentration environment. The total system sizes are 27788, 27789, 27778, and 27773 atoms for ICCG, ICCG (H218Y), ICCG (H218R), and ICCG (H218N) systems, respectively. For each kind of simulation, the energy of the system was then minimized using the steepest descent algorithm. This was followed by NVT equilibration for 2 ns with position restraints (force constant=1,000 kJ mol⁻¹ nm⁻²) on the all the heavy atoms of the complex at 343 K. V-rescale thermostat was used with a coupling constant of 0.1 ps.²¹ The system was then equilibrated in NPT condition with position restraint (force constant=1,000 kJ mol⁻¹ nm⁻²) on all heavy atoms of the complex at 1 bar. Parrinello-Rahman barostat with a coupling constant of 2 ps was used.²² This is followed by four sequential 200-ps MD simulations in NPT condition with reducing force constant for the position restraints on the complex, i.e.: 800, 600, 400, 200 kJ mol⁻¹ nm⁻². Then, the position restraint is released, and an MD simulation was performed for 50 ns with temperature annealing from 0 to 343K for the first 10 ns. Finally, the 10 production MD simulations were performed for 800 ns in NPT condition at 343 K for each system. Only the last 400 ns from each simulation were used for data analysis. For all simulations, hydrogen atoms were constrained with LINCs,²³ and the Leap-frog integrator with a step size of 2 fs was used

Calculation of Percent Frames with Dissociated PET₄ and Percent Frames with Catalytically Active Conformation

The PET₄ is described as dissociated from the active site when the distance between the S165 hydroxyl oxygen to the nearest C carbonyl atom of PET₄ (d1) is larger than 12 Å. The frames that follow this criterion were counted and divided by the total frames to obtain the percent frames with dissociated PET₄. To describe the catalytically active conformation of PET₄ in ICCG or ICCG variant, we calculated two more distances, i.e.: the distance between the amide nitrogen of Y95 (d2) and M166 (d3) to the nearest O carbonyl of PET₄. The PET₄ is described to be in a catalytically active conformation when i) d₁ is within 3.5 Å and ii) both d₂ and d₃ are within a threshold, ranging from 3 to 4 Å. The percentages of frames that follow these criteria were calculated. The reported percentage of catalytically relevant conformation is the average of these percentages of frames over the range of distances used in criteria ii). The averages and error bars of the percent frames were calculated from bootstrapping. The MD trajectories were concatenated and split into 80 groups. Bootstrapping was done 80 times resulting in 80 bootstrap samples. For each bootstrap sample, the 80 groups were randomly sampled with replacement and then concatenated to calculate the percent frames. The average and standard deviation of the percent frames were calculated across all bootstrap samples and plotted as average and error bar, respectively, in FIG. 5b and FIG. 5d.

### REFERENCES

1. Tournier, V. et al. An engineered PET depolymerase to break down and recycle plastic bottles. Nature 580, 216-219 (2020).
2. Chao, G. et al. Isolating and engineering human antibodies using yeast surface display. Nat. Protoc. 1, 755-768 (2006).
3. Shirke, A. N. et al. Stabilizing Leaf and Branch Compost Cutinase (LCC) with Glycosylation: Mechanism and Effect on PET Hydrolysis. Biochemistry 57, 1190-1200 (2018).
4. Martell, J. D. et al. A split horseradish peroxidase for the detection of intercellular protein-protein interactions and sensitive visualization of synapses. Nat. Biotechnol. 34, 774-780 (2016).
5. Chen, I., Dorr, B. M. & Liu, D. R. A general strategy for the evolution of bond-forming enzymes using yeast display. Proc. Natl. Acad. Sci. 108, 11399-11404 (2011).
6. Lam, S. S. et al. Directed evolution of APEX2 for electron microscopy and proximity labeling. Nat. Methods 12, 51-54 (2015).
7. Hong, V., Presolski, S. I., Ma, C. & Finn, M. G. Analysis and Optimization of Copper-Catalyzed Azide-Alkyne Cycloaddition for Bioconjugation. Angew. Chem. Int. Ed Engl. 48, 9879-9883 (2009).
8. Presolski, S. I., Hong, V. P. & Finn, M. g. Copper-Catalyzed Azide-Alkyne Click Chemistry for Bioconjugation. Curr. Protoc. Chem. Biol. 3, 153-162 (2011).
9. Mok, B. Y. et al. CRISPR-free base editors with enhanced activity and expanded targeting scope in mitochondrial and nuclear DNA. Nat. Biotechnol. 40, 1378-1387 (2022).
10. Walker, J. M. The Bicinchoninic Acid (BCA) Assay for Protein Quantitation, in The Protein Protocols Handbook (ed. Walker, J. M.) 11-14 (Humana Press, 1996). doi:10.1007/978-1-60327-259-9_3.
11. Nainar, S. et al. Temporal Labeling of Nascent RNA Using Photoclick Chemistry in Live Cells. J. Am. Chem. Soc. 139, 8090-8093 (2017).
12. Chindarkar, N. S. & Franz, A. H. Synthesis and characterization of biotin derivatives as multifunctional oligosaccharide tags. Arkivoc 2008, 21-33 (2008).
13. Zeng, W. et al. Substrate-Binding Mode of a Thermophilic PET Hydrolase and Engineering the Enzyme to Enhance the Hydrolytic Efficacy. ACS Catal. 12, 3033-3040 (2022).
14. Morris, G. M. et al. AutoDock4 and AutoDockTools4: Automated docking with selective receptor flexibility. J. Comput. Chem. 30, 2785-2791 (2009).
15. Trott, O. & Olson, A. J. AutoDock Vina: Improving the speed and accuracy of docking with a new scoring function, efficient optimization, and multithreading. J. Comput. Chem. 31, 455-461 (2010).
16. Wang, J., Wolf, R. M., Caldwell, J. W., Kollman, P. A. & Case, D. A. Development and testing of a general amber force field. J. Comput. Chem. 25, 1157-1174 (2004).
17. Abraham, M. J. et al. GROMACS: High performance molecular simulations through multi-level parallelism from laptops to supercomputers. SoftwareX 1-2, 19-25 (2015).
18. Maier, J. A. et al. ff14SB: Improving the Accuracy of Protein Side Chain and Backbone Parameters from ff99SB. J. Chem. Theory Comput. 11, 3696-3713 (2015).
19. The PyMOL Molecular Graphics System, Version 2.0 Schrödinger, LLC.
20. Jorgensen, W. L., Chandrasekhar, J., Madura, J. D., Impey, R. W. & Klein, M. L. Comparison of simple potential functions for simulating liquid water. J. Chem. Phys. 79, 926-935 (1983).
21. Bussi, G., Donadio, D. & Parrinello, M. Canonical sampling through velocity rescaling. J. Chem. Phys. 126, 014101 (2007).
22. Parrinello, M. & Rahman, A. Polymorphic transitions in single crystals: A new molecular dynamics method. J. Appl. Phys. 52, 7182-7190 (1981).
23. Hess, B., Bekker, H., Berendsen, H. J. C. & Fraaije, J. G. E. M. LINCS: A linear constraint solver for molecular simulations. J. Comput. Chem. 18, 1463-1472 (1997).
24. Boder, E. T. & Wittrup, K. D. Yeast surface display for screening combinatorial polypeptide libraries. Nat. Biotechnol. 15, 553-557 (1997).

### Example 2. Probe Association by Phenol Coupling

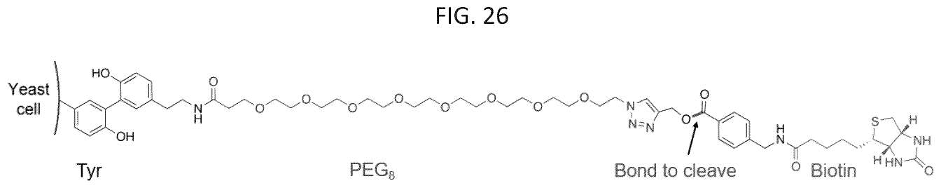

A biotin-ester probe attachment strategy using UV activated, ruthenium-catalyzed, or horseradish peroxidase (HRP) phenol coupling is shown in FIG. 26. The described probe attachment strategy may advantageously be used at a pH of 4 or below.

S. cerevisiae cells containing the Round 3 LCC sorted library were diluted 1:9 in SGCAA media to induce surface expression. The culture was allowed to grow for 20 hours to saturation. 5 mL cultures were spun down for 1.5 minutes at 4,300×g at 24° C. The supernatant was removed and the cells were washed once with PBS pH 7.4. The cells were then suspended in 2 mL of PBS pH 7.4 and OD600 was measured to determine cell density. Aliquots of 60 million cells were spun down for 1 minute at 13000×g at 24° C. The supernatant was removed, and the cell pellet was resuspended in 100 μL of reaction mixture containing 10 mM MES buffer pH 7.4, 6.2, 5.5, or 4.2 supplemented with 10 mM MgCl2, 10% DMSO, 10 μM biotin-ester-peg-phenol probe (shown in FIG. 26), 1 mM Ammonium Persulfate (APS), and 1 μM Ru(bpy)3. The suspended yeast samples were then irradiated on their side, 2.5 inches from a 456 nM Kessil lamp set to 50% intensity while shaking vigorously over ice for 5 minutes. After irradiation, the cells were spun down for 1 minute at 13000×g at 24° C. The supernatant was removed, and cells were washed twice with PBS-B at the corresponding pHs listed above. The cell pellet was then suspended in 1 mL of PBS-B at corresponding pH and incubated for 2 hours stationary at room temperature to allow for probe cleavage. The cells were spun down, the supernatant removed, and washed once with PBS-B at corresponding pH. Separately, a solution containing 1:100 μL anti-c-myc antibody (EMD Millipore Co., AB3253) in PBS-B pH at corresponding pH was prepared. The pellet following the wash was resuspended in 50 μL of the anti-c-myc antibody solution and incubated in a thermomixer set to 24° C., 800 rpm shaking for 30 minutes. The cells were washed twice with PBS-B at corresponding pH. Separately, a solution containing 1:100 AlexaFluor™ 647 Goat anti-chicken IgY (H+L) (Invitrogen, A21449) and 1:100 streptavidin-phycoerythrin (sAv-PE, Invitrogen, S866) in PBS—at corresponding pH was prepared and added to the washed pellet. The pellet was resuspended and incubated in a thermomixer set to 24° C., 800 rpm shaking for 30 minutes. The cells were washed twice with PBS-B at corresponding pH and analyzed via flow cytometry.

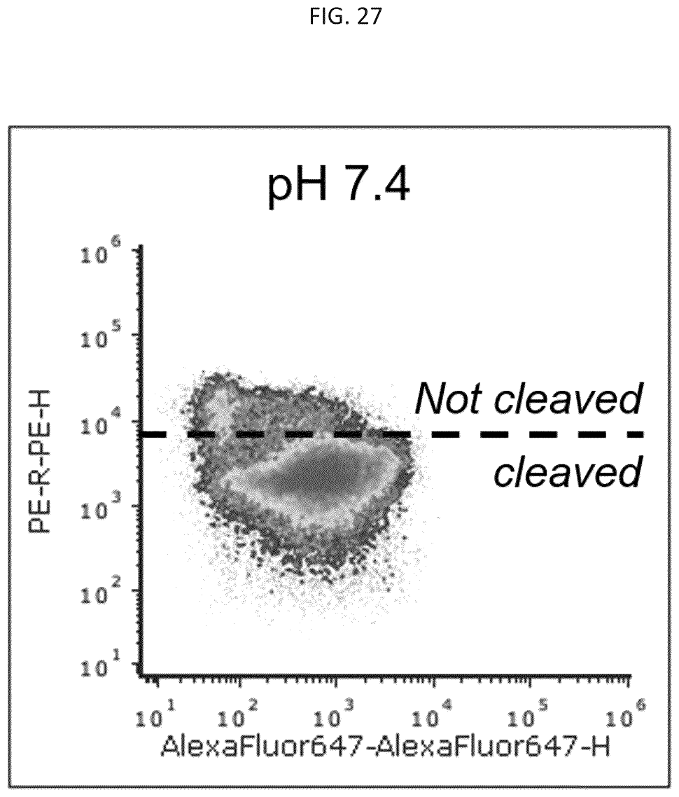

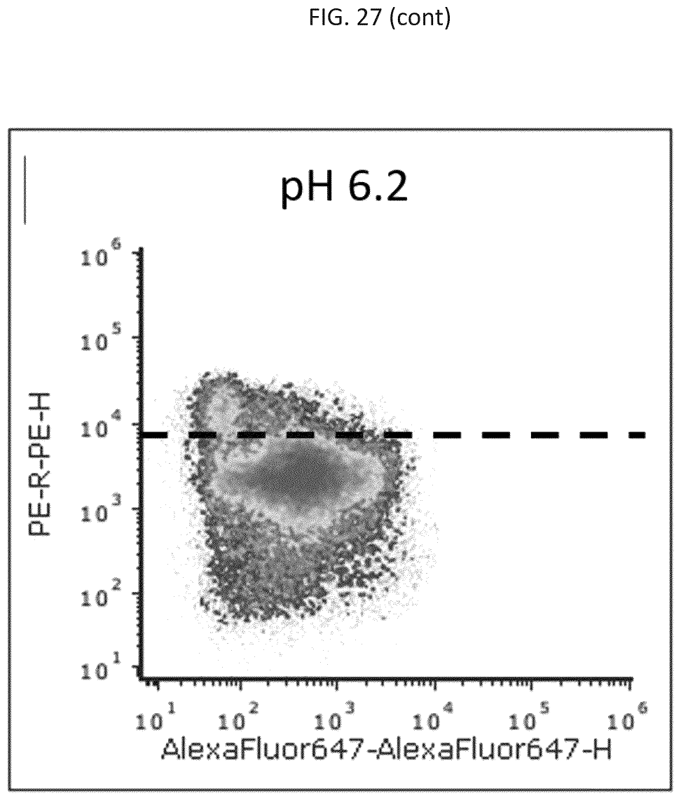

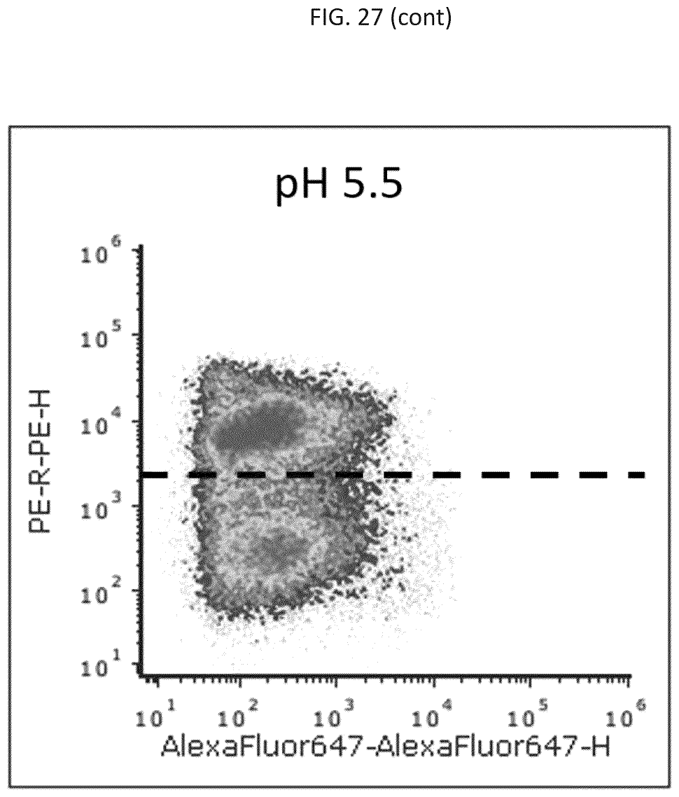

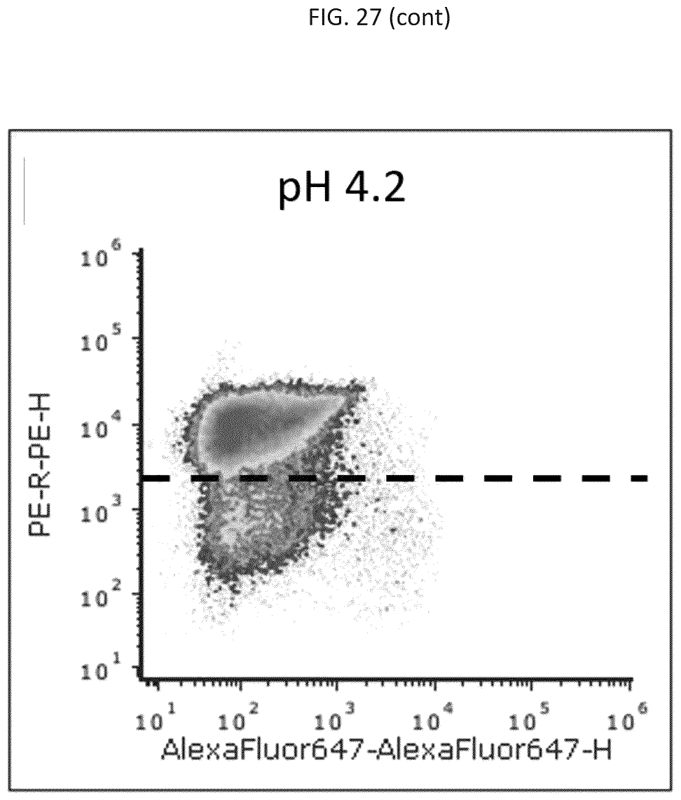

The same sorting protocol was used as described above for the amine associated probes. FIG. 27 shows flow cytometry analysis of round 3 sorted yeast enzyme expression and hydrolysis activity against FIG. 26 probe on the yeast surface for at a pH of a) 7.4, b) 6.2, c) 5.5, and d) 4.2. Enzyme expression is characterized on the x-axis by myc-epitope tag staining and activity is characterized on the y-axis via fluorescent streptavidin (phycoerythrin) labeling of biotins.

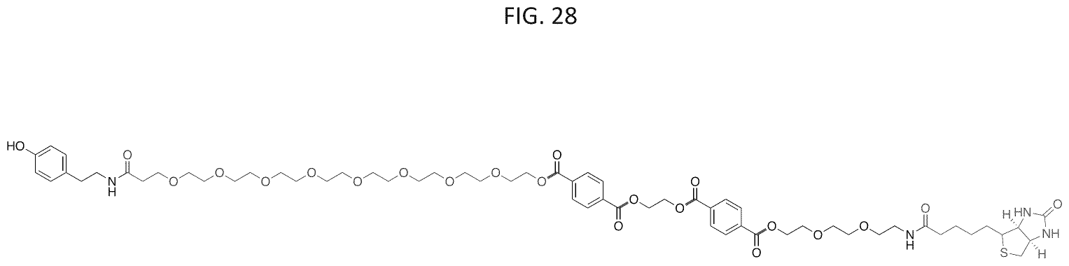

FIG. 28 depicts a synthetic probe featuring two PET units in the linker, with four aromatic ester functional groups. One end of the probe consists of biotin, which is used for fluorescence staining of yeast cells (high fluorescence indicates low activity). The other end features a phenol, which is used for coupling to proteins on the yeast surface. Polyethylene glycol spacers are included to minimize steric hinderance.

### Example 3. Alternative Fusion Protein Labels

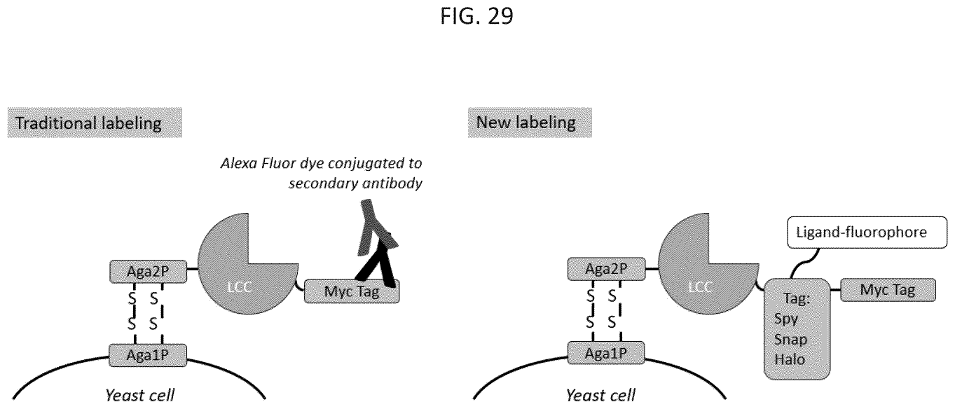

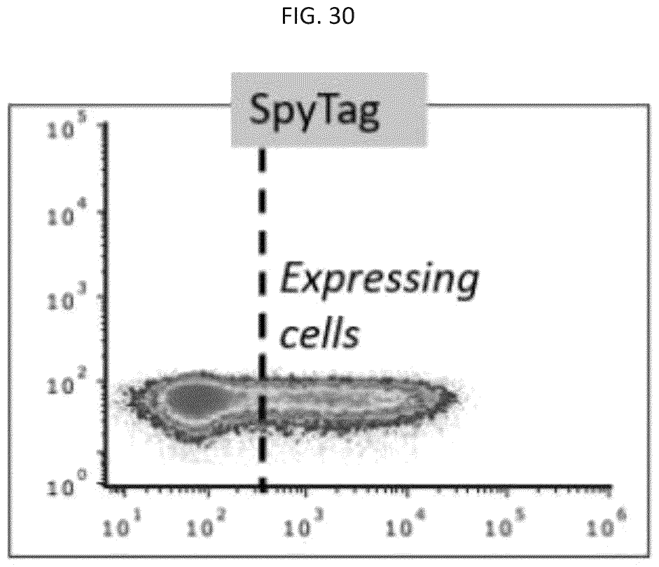

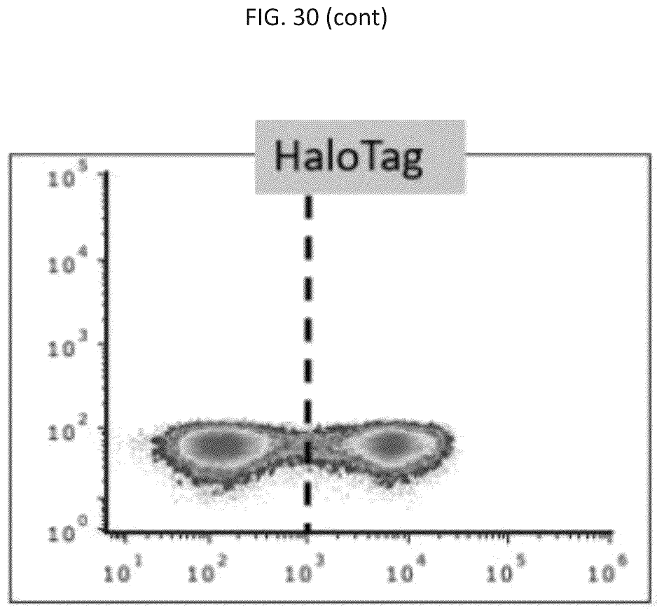

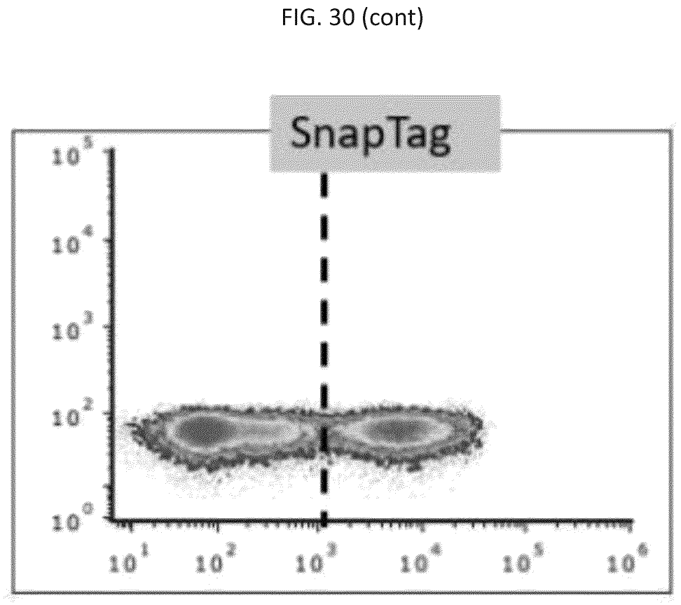

FIG. 29 illustrates that alternative fusion protein may be used, e.g., spy, halo, and snap tags alone or in combination with another tag, e.g., a myc tag.

HaloTag Ligand—Biotin was purchased from Promega (G8282) and used to label cells by diluting 1:1000 into PBS-B. 50 μL of diluted ligand was incubated with cells for one hour in a thermomixer set to 24° C., 800 rpm shaking for 1 hour. The cells were washed twice with PBS-B pH 7.4. Separately, a solution containing 1:100 streptavidin-phycoerythrin (sAv-PE, Invitrogen, S866) in PBS—at pH 7.4 was prepared and added to the washed pellet. The pellet was resuspended and incubated in a thermomixer set to 24° C., 800 rpm shaking for 30 minutes. The cells were washed twice with PBS-B at corresponding pH and analyzed via flow cytometry.

Snap-Biotin was purchased from New England BioLabs (S9110S) and was diluted to 10 μMinto PBS-B pH 7.4. 50 μL of diluted ligand was incubated with cells for one hour in a thermomixer set to 24° C., 800 rpm shaking for 1 hour. The cells were washed twice with PBS-B pH 7.4. Separately, a solution containing 1:100 streptavidin-phycoerythrin (sAv-PE, Invitrogen, S866) in PBS—at pH 7.4 was prepared and added to the washed pellet. The pellet was resuspended and incubated in a thermomixer set to 24° C., 800 rpm shaking for 30 minutes. The cells were washed twice with PBS-B at corresponding pH and analyzed via flow cytometry.

SpyCatcher3-CYS was purchased from Bio-Rad (TZC025CYS). To make the SpyCatcher-fluorescein conjugate, 20 μL of the undiluted SC3-CYS was reduced by using 30 μL of 50 mM TCEP and 100 μL of PBS pH 5 with 10 mM EDTA (PBS-E) for 2 hours at 37° C. with 300 rpm shaking in a thermomixer. The reduced protein was then purified using an Amicon 3K ultracentrifuge device by diluting the reduced protein mixture to 500 μL with PBS-E pH 7.4 then loading it on the centrifugal device. 6 spins were performed at 14000×g for 6 minutes at 4° C., removing the flow through and diluting the sample to 500 μL using PBS-E, pH 7.4 after each spin. The purified reduced protein was then reacted with 5 equivalence of fluorescein maleimide in a thermomixer set to 37° C. with 300 rpm shaking for 2 horns and 45 minutes. The mixture was then incubated in a 4° C. refrigerator overnight. The conjugate was then purified using an Amicon 3K ultracentrifuge device by diluting the reduced protein mixture to 500 μL with PBS-E pH 7.4 then loading it on the centrifugal device. 6 spins were performed at 14000×g for 6 minutes at 4° C., removing the flow through and diluting the sample to 500 μL using PBS-E, pH 7.4 after each spin. The final concentration was determined via UV-Vis absorbance at 280 and 495 nm.

To label SpyTag-displaying cells, samples were suspended in a mixture of 1 μM Spy-Catcher-Fluorescein in PBS-B pH 7.4. 50 μL of diluted SC-fluorescein was incubated with cells for one hour in a thermomixer set to 24° C., 800 rpm shaking for 1 hour. The cells were washed twice with PBS-B and analyzed via flow cytometry.

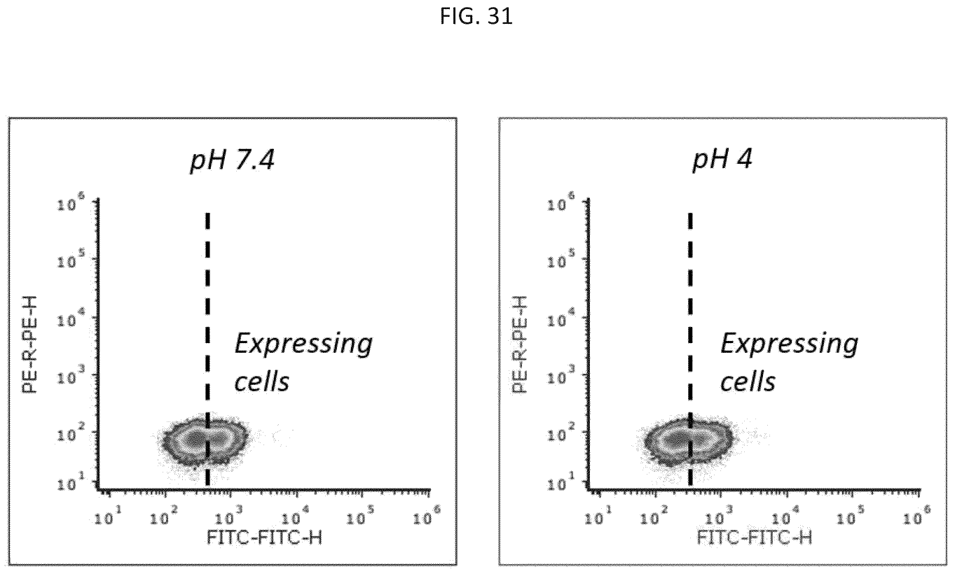

FIG. 31 was prepared by using the same SpyCatcher-fluorescein labeling procedure, but also including a sample where Citrate-BSA buffer, pH 4 was used in place of PBS-B pH 7.4.

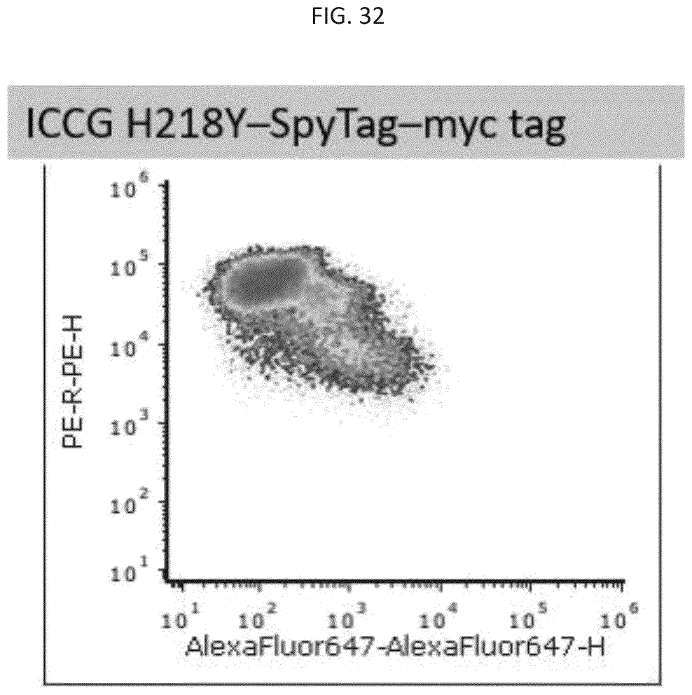

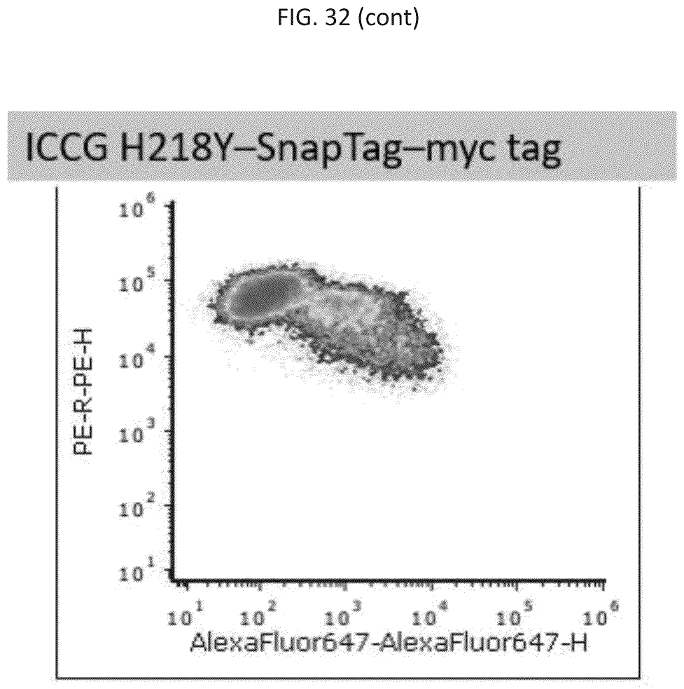

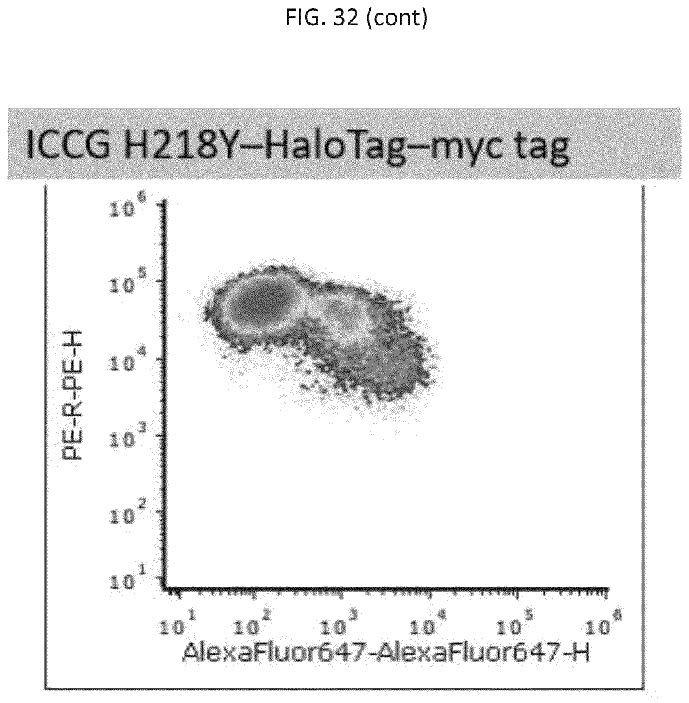

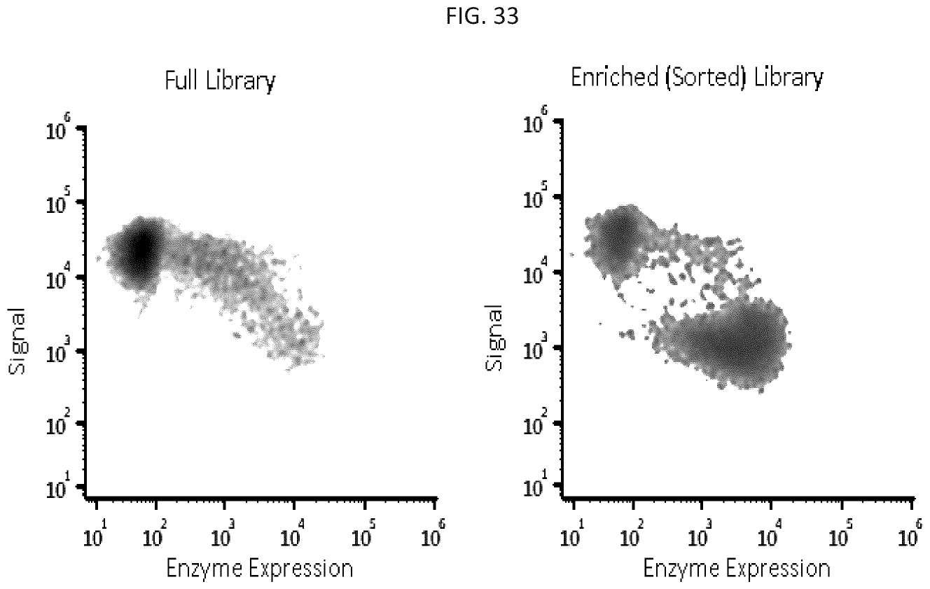

FIG. 32 experiments were done using yeast displaying Aga2p-LCC ICCG-H183Y-Epitope tag-Myc Tag where the Epitope tag is either the SnapTag, SpyTag, or HaloTag. The yeast were labeled as described beforehand using the amine-linked cleavable probe and Myc staining. The purpose here was to ensure that the additional epitope tags didn't interfere with the activity of the enzyme.

FIG. 33 depicts flow cytometry results in which enzyme expression was detected by staining of the myc epitope tag.

### Example 4. Probes with Lactic Acid Polymer Substrates

Probes with alternative polymer substrates may be incorporated into the probe. The present Example illustrates the incorporation of a lactic acid polymer substrate.

### Synthesis of 4b

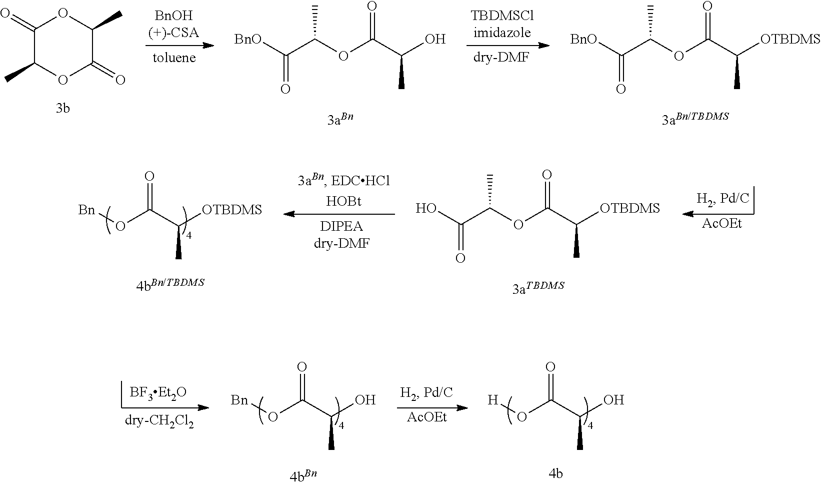

Synthesis of 4b from 3b was carried out using previously reported syntheses.¹⁻⁵

Synthesis of 3a^{Bn}: L-Lactide (3b; 5.01 g, 34.7 mmol), benzyl alcohol (BnOH; 4.3 mL, 41.5 mmol), and (+)-10-camphorsulfonic acid ((+)-CSA; 44.6 mg, 0.17 mmol) were dissolved in toluene (21 mL) in a 50 mL round-bottom glass flask. The resultant mixture was stirred at 80° C. for 8 h and then the solution was washed with 0.2 M NaHCO₃ aq. The organic phase was dried over Na₂SO₄, filtrated, and concentrated under reduced pressure. The crude product was purified by silica-gel column chromatography (hexane:AcOEt=1:1) to give 3a^{Bn} as colorless oil (7.32 g, 29.0 mmol; 83% yield). NMR

Synthesis of 3a^{Bn/TBDMS}: Compound 3a^{Bn} (3.99 g, 15.8 mmol) and imidazole (2.71 g, 39.8 mmol) was dissolved in dry DMF (24 mL) in a 50 mL round-bottom glass flask under N₂ and then tert-butyldimethylchlorosilane (TBDMSCl; 4.08 g, 27.1 mmol) was added to the flask. The resultant mixture was stirred at r.t. overnight. Sat. NaHCO₃ aq. was added to the solution and then the product was extracted with hexane. The organic phase was dried over Na₂SO₄, filtrated, and concentrated under reduced pressure. The crude product was purified by silica-gel column chromatography (hexane:AcOEt=1:0 to 1:1) to give 3a^{Bn/TBDMS} as colorless oil (5.16 g, 14.0 mmol; 89% yield). NMR

Synthesis of 3a^{TBDMS}: Compound 3a^{Bn/TBDMS} (615 mg, 1.68 mmol) and Pd/C (5%) (74.3 mg) was dissolved in AcOEt (3 mL) in a 25 mL round-bottom glass flask under H₂. The resultant mixture was stirred at r.t. for 19 h. The black suspension was filtered through a Celite plug with AcOEt. The filtrate was concentrated under reduced pressure. The crude product was purified by silica-gel column chromatography (AcOEt) to give 3a^{TBDMS} as colorless oil (251 mg, 14.0 mmol; 54% yield). NMR

Synthesis of 4b^{Bn/TBDMS}: Compounds 3a^{TBDMS} (251 mg, 0.90 mmol) and 3a^{Bn} (264 mg, 1.04 mmol) were dissolved in dry DMF (3 mL) in a 50 mL round-bottom glass flask under N₂ and then the solution was cooled to 0° C. A mixture of 1-(3-dimethylaminopropyl)-3-ethylcarbodiimide hydrochloride (EDC·HCl; 195 mg, 1.02 mmol), 1-hydroxybenzotriazole (147 mg, 1.09 mmol), and dry DMF (3 mL) were added dropwise to the 50 mL flask, and then N,N-diisopropylethylamine (DIPEA; 340 μL, 1.99 mmol) was added dropwise to the same flask. The resultant mixture was stirred at r.t. for 19 h. AcOEt (7 mL) was added to the solution and then the resultant solution was washed with brine (2 mL×4). The organic phase was dried over Na₂SO₄, filtrated, and concentrated under reduced pressure. The crude product was purified by silica-gel column chromatography (hexane:AcOEt=3:1) to give 4b^{Bn/TBDMS} as colorless oil (321 mg, 0.62 mmol; 69% yield). NMR

Synthesis of 4b^{Bn}: Compound 4b^{Bn/TBDMS} (92.6 mg, 0.18 mmol) was dissolved in dry CH₂Cl₂ (3 mL) in a 50 mL round-bottom glass flask under N₂. BF₃·Et₂O (110 μL, 0.87 mmol) was added dropwise to the solution at 0° C. The resultant mixture was stirred at r.t. for 4 h. The solution was washed with a mixture of sat. NaHCO₃ aq. and brine (3:1). The resulted organic layer was separated and washed with brine. The organic phase was dried over Na₂SO₄, filtrated, and concentrated under reduced pressure. The crude product was purified by silica-gel column chromatography (hexane:AcOEt=1:1) to give 4b^{Bn} as colorless oil (67.4 mg, 0.17 mmol; 83% yield). NMR

Synthesis of 4b: From compound 4b^{Bn} (11.7 mg, 29.5 μmol), pure lactic acid tetramer (4b) was obtained as colorless oil (8.6 mg, 28.3 μmol; 96% yield) using the same method for the deprotection of 3a^{Bn/TBDMS}. NMR

While biologically relevant hydrophilic molecules rarely interact with hydrophobic compounds and surfaces in water owing to effective hydration, the hydrophobic cavity of a polyaromatic capsule formed through coordination-driven self-assembly can encapsulate hydrophilic oligo(lactic acid)s in water with relatively high binding constants (up to Kₐ=3×10⁵ M⁻¹). The unusual host-guest behavior is caused by enthalpic stabilization through multiple CH-π and hydrogen-bonding interactions. The polyaromatic cavity stabilizes hydrolyzable cyclic di(lactic acid) and captures tetra(lactic acid) preferentially from a mixture of oligo(lactic acid)s even in water.⁵ Herein, the stereospecific polyamide 4b^{Bn} is coupled with biotin, followed by deprotection of the amide, and finally coupled to a phenol probe design as illustrated below.

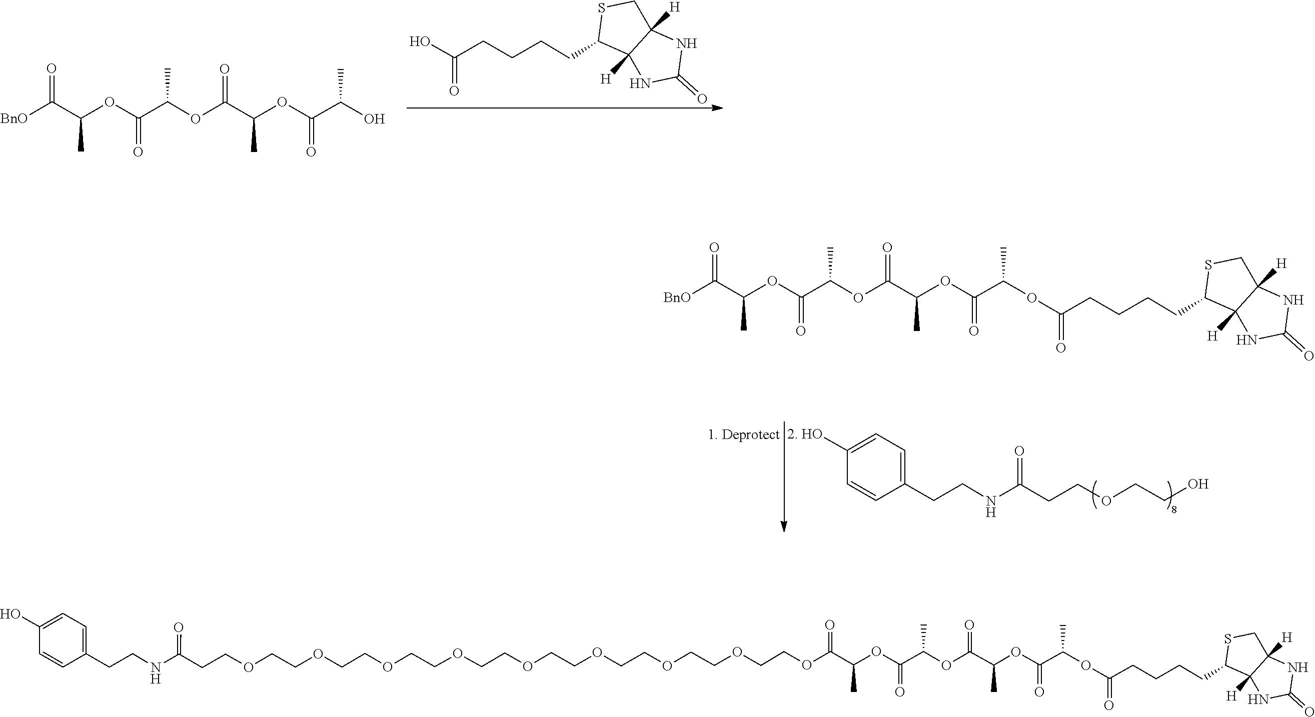

The scheme above depicts a general plan for synthesizing a probe containing an oligomer of poly-lactic acid in the linker, with biotin attached to one end and a phenol attached to the other end to allow coupling to the cell surface. In the first step, an alcohol on a poly-lactic acid oligomer is attached to a carboxylic acid on biotin, forming an aliphatic ester linkage. In the second step, a protecting group is removed from an ester on the other end of the probe, revealing a carboxylic acid, which is in turn coupled to an alcohol on a phenol-containing molecule, forming another new aliphatic ester linkage and yielding the final product.

### REFERENCES

[1] H. Zhu, X. Xu, W. Cui, Y. Zhang, H. Mo, Y.-M. Shen, J. Polym. Sci. Part A: Polym. Chem. 2011, 49, 1745-1752.
[2] M. Singh, N. P. Argade, Synthesis 2012, 44, 3797-3804.
[3] T. I. Al-Warhi, H. M. A. Al-Hazimi, A. El-Faham, J. Saudi Chem. 2012, 16, 97-116.
[4] B. van Genabeek, B. F. M. de Waal, M. M. J. Gosens, L. M. Pitet, A. R. A. Palamans, E. W. Meijer, J. Am. Chem. Soc. 2016, 138, 4210-4218.
[5] Kusaba. Et al. Angew. Chem. Int. Ed. 2018, 57,3706-3710.

### Example 5

While the initial platform worked as intended, there was still a desired to find LCC mutants with even higher activity. Acid tolerance is an attribute that many LCC degrading enzymes lack but is desirable. The initial selection (published in JACS 2023) was performed at pH 5.5 to induce some pressure via an acidic environment, but the resultant winning mutant did not exhibit a large increase in acid tolerance when expressed recombinantly and assessed via a PET film degradation assay.

Two main adaptations need to be made to make the platform functional below pH 5.5. The first of which is the way that enzyme expression levels were monitored. The initial platform utilized a c-myc tag fused to the C-terminus of the protein followed by fluorescent antibody staining to tag expressing cells. However, because antibodies rely on non-covalent interactions that are susceptible to disruption at low pH, antibody staining failed to work sufficiently at pH 4. Secondly, the PET mimicking probe, harboring an alkyne handle, was covalently attached via a CuAAC reaction to an NHS ester-PEG₈-Azide functionalized yeast cell surface. Similar to the antibodies, this technique worked at pH 5.5, but failed at pH 4.

### Acid-Tolerant Modifications to the Yeast Display Platform

To overcome the acid-intolerant issues in the first platform, a new platform was developed that would enable the selection process to occur at pH 4 aside from a one-minute labeling step at pH 5. Several covalent epitope tags were screened for their compatibility with pH 4 buffers. The SpyTag/SpyCatcher system offered the best solution as it is small in size to limit protein folding disruption and can withstand subsequent labeling steps at an acidic pH. SpyTag is a small, 13 amino acid tag that reacts with a specific lysine residue on SpyCatcher to form an isopeptide bond, and a cysteine containing SpyCatcher is commercially available to enable AlexaFluor-maleimide dye bioconjugation.¹ Additionally, we chose to use oxidative phenol coupling to attach to the probe to the cell surface instead of the copper click. It was serendipitously discovered in the Ru(II) proximity labeling work described in Chapter 3 that this chemistry works very well at pH 4. Ultimately horseradish peroxidase (HRP) was chosen to catalyze the phenol coupling to tyrosine residues on the cell surface over a Ru photocatalyst to avoid photobleaching the Spy-Catcher dye conjugate. Due to unwanted side reactions with the dye attached to SpyCatcher under pH 4 conditions, the HRP labeling step was performed at pH 5 for only one minute, and the rest of the selection continued at pH 4.

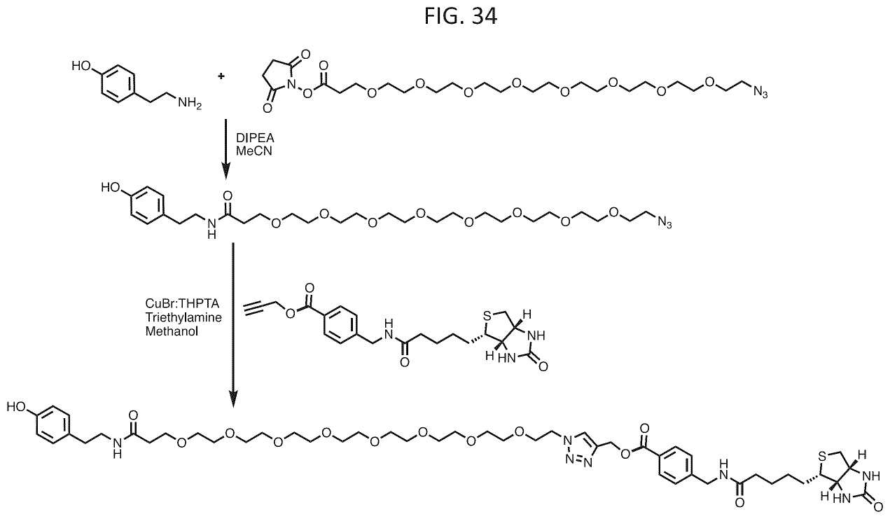

To validate the platform, a plasmid was cloned containing ICCG^{H218Y} with SpyTag fused to the C-terminus of the protein. Additionally, a phenol version of the biotin ester alkyne probe used in the first sorting campaign was synthesized by further reacting the alkyne probe with azide-PEG₈ tyramine to afford the probe with a phenol coupling handle (FIG. 34). We validated the new labeling protocols by first labeling the cells with

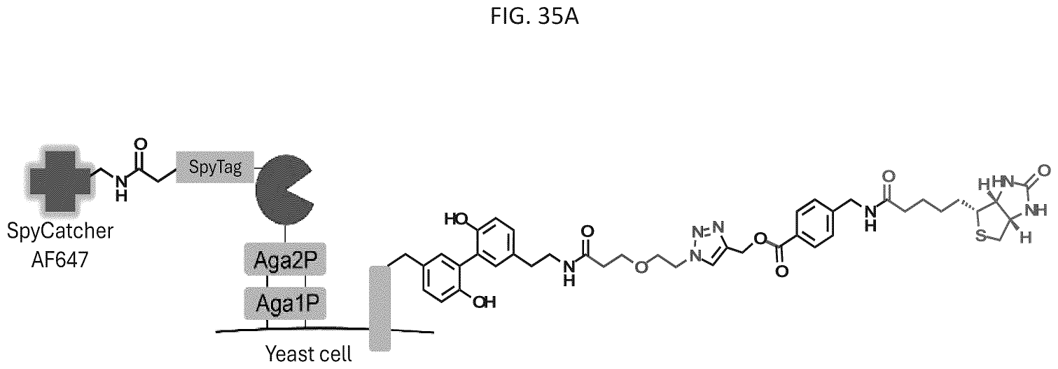

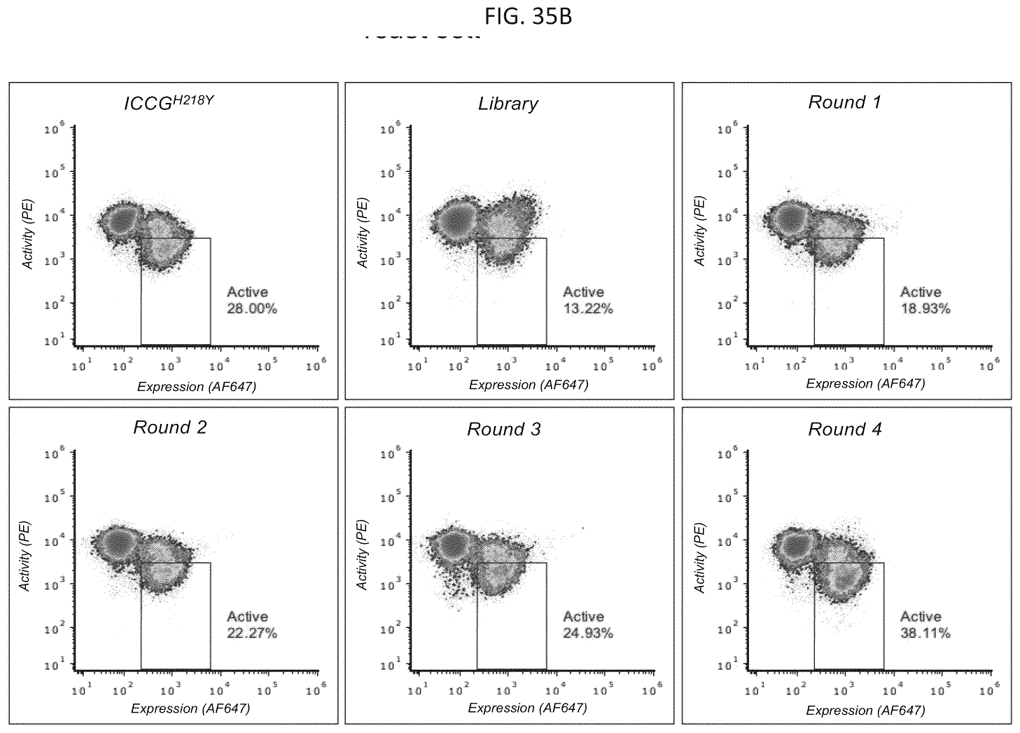

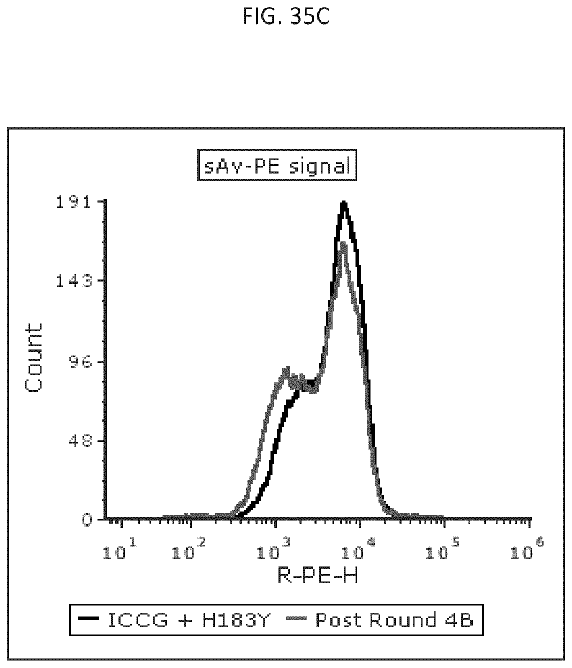

SpyCatcher003-Cys functionalized with AF-647 maleimide. Next, cells were exposed to HRP catalyzed phenol labeling with the biotin ester phenol probe at pH 5 for one minute. Immediately after this one-minute labeling, the pH of the solution was lowered to pH 4 where it remained for a two-hour cleavage period and subsequent sAv-PE staining. We observed a subtle decrease in fluorescence of the expressing, SpyTag-positive cells compared to the non-expressing cells, indicating that ICCG^{H183Y} had some activity under these conditions (FIG. 35B, upper left panel).

A library was then created using ICCG^{H218Y} as the parent enzyme using error-prone PCR and transformed into yeast to produce a 4.5×10⁷-member library with an average of 1.85 amino acid mutations per gene. This library underwent 5 rounds of sorting using the aforementioned labeling techniques, keeping only the most active mutants each round. After 4 rounds of sorting the relative proportion of yeast displaying activity against the probe increased as evidenced by decreased sAv-PE signal (FIG. 35B). Additionally, the level of probe cleaved also increased as shown by the lowest fluorescence of expressing cells dropping in the post round 4 yeast compared to the parent ICCG^{H183Y} (FIG. 35C).

After rounds 4 and 5, sequencing of 80 mutants was performed for each round. When comparing both rounds of sequencing, C238R was the most common in both sets of sequencing data, while C238Y was also found. C238 is one of the cysteine residues that make up the LCC “ICCG” quadruple mutant. The double cysteine mutation in ICCG relative to LCC was installed to increase thermostability, and the removal of this disulfide bond may indicate that perhaps there is trade-off between thermostability and acid tolerance. An A97V/T mutation was also very prevalent in the post round 5 mutants. Although this residues is not directly in the active site cavity, this position has been hypothesized to interact with the substrate, and mutating the 97 position to threonine was found to increase activity of LCC.² Due to the poor structure similarity of the probe used for evolution and actual PET polymer, it is possible that the A97V mutation increases activity towards our probe, but not the desired substrate, PET plastic. Another interesting mutation away from ICCG was an I243T mutation. This mutation has also been reported in the past, but in the context of WT LCC where an F243T mutation lead to a 175% increase in activity on PET nanoparticles.² Additionally, a new double cysteine mutant, Y61C+R151C, was present in the post-round 5 sequencing, albeit in low frequency. While a novel disulfide bond seems improbable based on the current crystal structure of ICCG, it is possible that the secondary structure of this particular mutant is slightly shifted to accommodate a new disulfide bond. Finally, an L117S mutation piqued our interest because the leucine is positioned in a relatively hydrophobic pocket, and mutation to serine would increase hydrophilicity.

Several mutants were selected for further analysis using monoclonal yeast populations and recombinant enzyme purified from E. coli for subsequent PET film experiments. From this point forward, mutants will be referred to using an alpha-numeric code (Table 1).

TABLE 1
Select mutations found in sequences of post-round 4 or 5 sorting.

| Round of sequencing | Mutant Name | Mutations |
|---|---|---|
| Round 4 | A14 | S69N, L117S, C238R |
| Round 4 | C2 | C238Y |
| Round 4 | C5 | A97V, L117S |
| Round 5 | A1 | A97T, N266S, L66P, Y61F |
| Round 5 | A6 | A97V, T51P |
| Round 5 | B16 | Y61C, R151C |
| Round 5 | D4 | I243T |

Mutations of interest are reported in bold font.

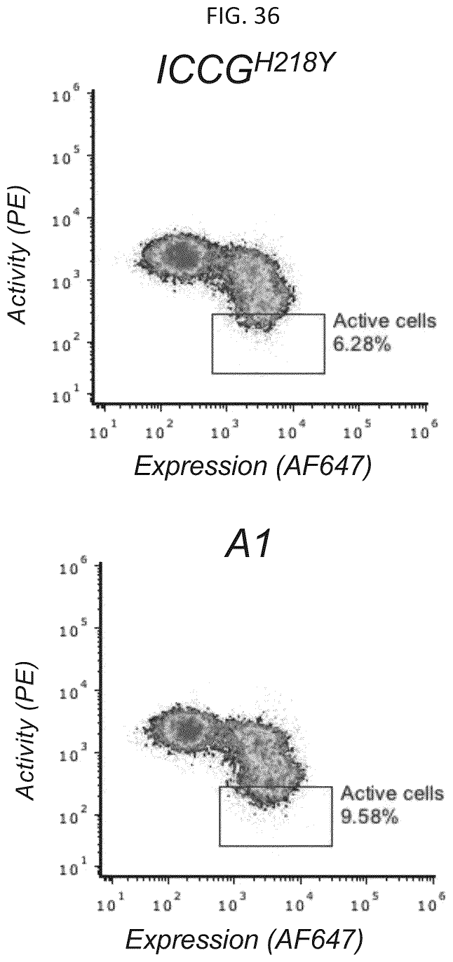

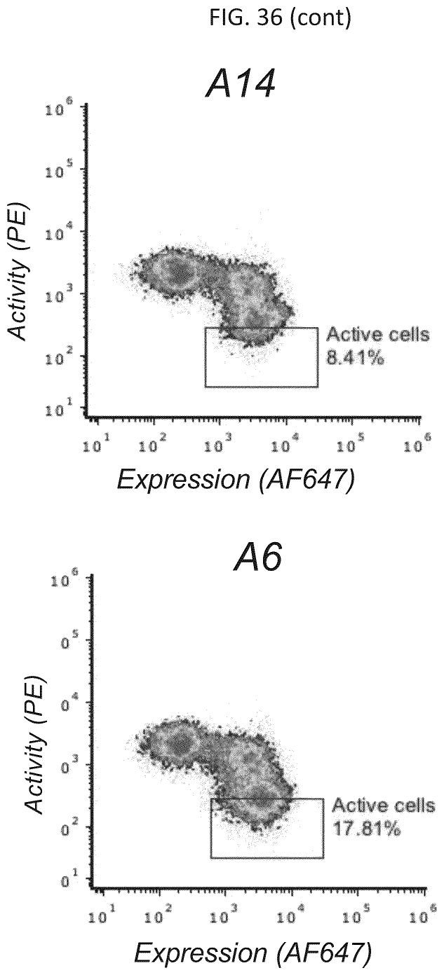

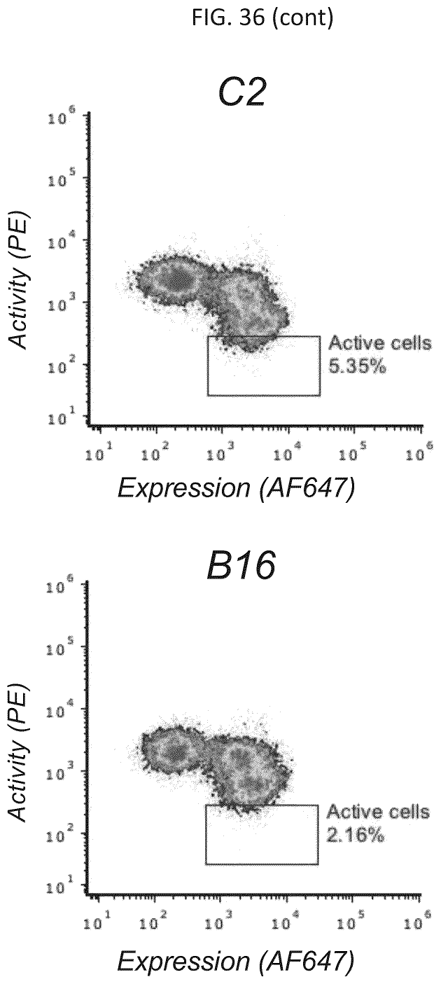

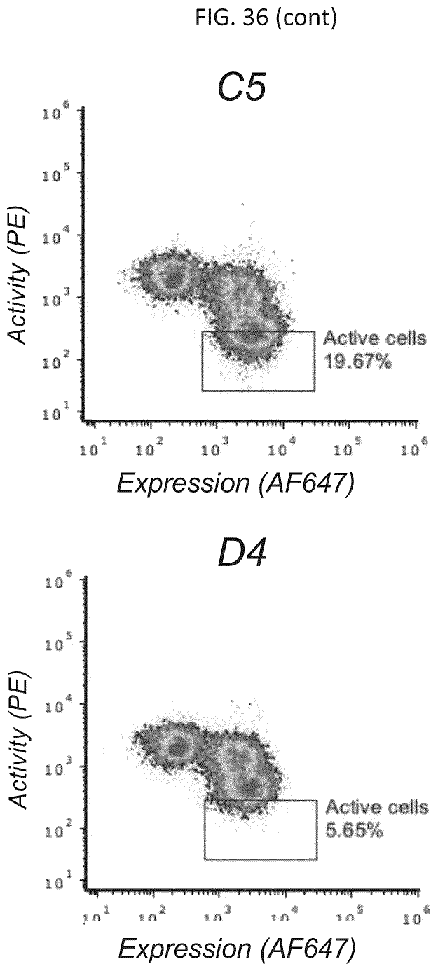

We first evaluated the activity of the mutants on the yeast cell surface in monoclonal yeast populations to test their activity to cleave the biotin ester phenol probe. The yeast were exposed to the same labeling conditions used during the selection process and analyzed via flow cytometry (FIG. 36). Mutants C5 and A6 had the highest activity against the biotin ester phenol probe, with 19.7 and 17.8% of cells, respectively. They had very high activity, defined as the percentage of cells having the same sAv-PE fluorescence as unstained cells, indicating almost complete cleavage of the probe. Interestingly, both C5 and A6 have A97V mutations with different co-mutations. Mutants A14, C2, A1, and D4 have similar activity to the parent ICCG^{H218Y} while mutant B16 seems to have worse activity against this probe, as evidenced by higher sAv-PE fluorescence of the expressing population compared to the parent enzyme. We next sought to evaluate the activity of these mutants against amorphous PET film.

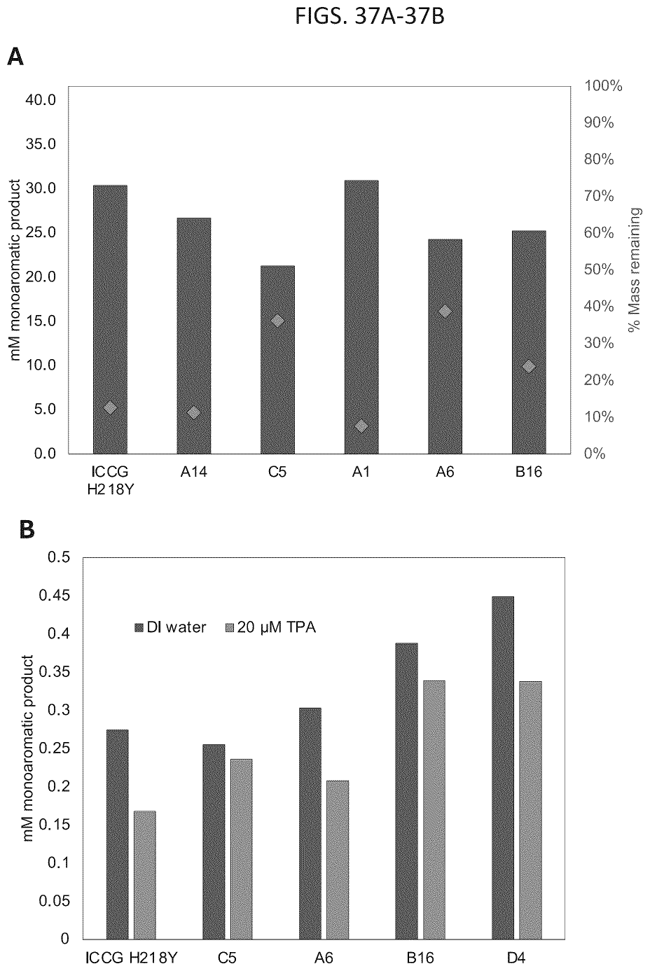

Initial tests with recombinant enzyme included an 8 mg PET film degradation assay using 500 nM enzyme at 70° C. and 500 mM HEPES buffer, pH 8. A solution of pH 8 was initially tested as this is a commonly used starting pH for PET degrading enzymes to counteract the acid accumulation over time. A temperature of 70° C. was used because it is close to the glass transition temperature of PET where the amorphous regions of the plastic become more accessible to the enzyme, increasing the rate of degradation. After 16.5 hours the solution phase was analyzed via HPLC to determine total concentration of mono-aromatic product (i.e. terephthalic acid (TPA), mono(2-hydroxyethyl) terephthalate (MHET), or bis(2-hydroxyethyl) terephthalate (BHET)) and the remaining plastic film was massed to determine the percent mass remaining compared to the starting mass of 8 mg (FIG. 37A). Using these conditions, we observed that mutants A14 and A1 had similar levels of activity compared to the parent ICCG^{H218Y}. Mutants C5, A6, and B16 were found to have worse activity, with 36, 39, and 24% of mass remaining, respectively, compared to 13% mass remaining for ICCG^{H218Y}.

We next tested the activity of these mutants under acidic or non-buffered conditions since that is the characteristic we were aiming to evolve for. To test if the mutants were still active in the presence of terephthalic acid, which is a product of PET degradation and lowers the pH of the solution, we performed film degradation using a 20 μM TPA solution, pH 4. Additionally, to see if an un-buffered solution could be employed, which would decrease the barrier to using enzymes industrially for PET degradation, we used deionized water. Mutants B16 and D4 showed more product release under both conditions compared to parent ICCG^{H218Y} while mutants A6 and C5 had similar to marginally better activity under these conditions (FIG. 37B). While the total amount of product released corresponds to less than 1% of total possible product, it is encouraging that the B16 and D4 mutants are capable of enzymatic degradation under these challenging conditions. It is also interesting that these mutants did not out-perform parent ICCG^{H218Y} under high buffering, high pH conditions but did under conditions more similar to the selection.

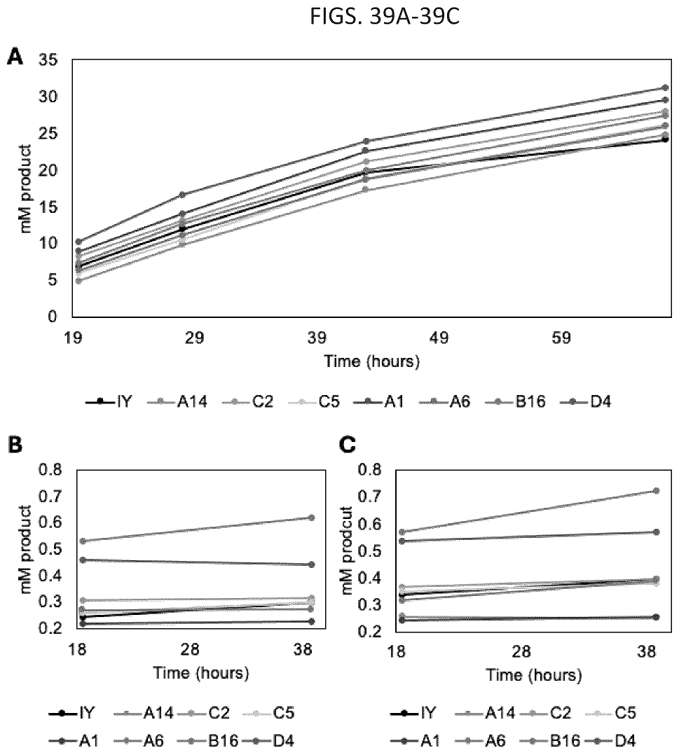

We also tested the activity of the mutants at a lower temperature, 55° C., that is below the glass transition temperature of PET which has been demonstrated to enable full degradation of PET, but at a slower rate. We hypothesized that because the selection itself was performed at room temperature, it is possible that mutants which were selected for acid-tolerance lost the thermostability afforded by the ICCG mutations. We tested a potassium phosphate buffer at pH 8, a 20 μM TPA solution at pH 4.2, and deionized water as the solvent with 500 nM enzyme and an 8 mg PET film disk at 55° C. After 67.5 hours of incubation at 55° C., we observed that all mutants tested yielded higher product formation than the parent ICCG^{H218Y}(IY). This differs from the pH 8 data in FIG. 37 at 70° C., suggesting that the mutants from the sorted yeast were more active than parent, but not at increased temperatures. The data using a TPA solution or DI water at 55° C. was similar to the 70° C. data with mutants B16 and D4 producing the most aromatic monomers. Additionally, mutant B16 remained active during the two time points tested while D4 did not, suggesting that after the 18-hour time point, D4 was deactivated while B16 as well as several other mutants continued to degrade the film (FIG. 39).

### Apply the Platform to Increase Acid Tolerance in Polymer-Degrading Enzymes.

Having validated the performance of our unique evolution platform at pH 4, the next objective is to use it to screen millions of mutants of polyester-degrading enzymes (with mutations throughout the structure) for enhanced activity under acidic conditions, assess which mutations are enriched, and test whether these mutations enhance activity at pH 4 on the target polymer material. Toward this goal, I propose that a promising starting point for enzyme evolution is the enhanced-activity enzyme, “ICCG+Y”, because it exhibits superior activity to ICCG on solid PET. In parallel, a variety of other polymer-degrading enzymes—with varying degrees of homology to ICCG (referenced above)—will be incorporated into the platform, subjected to mutagenesis, and investigated for activity under acidic conditions using our ultrahigh-throughput workflow.

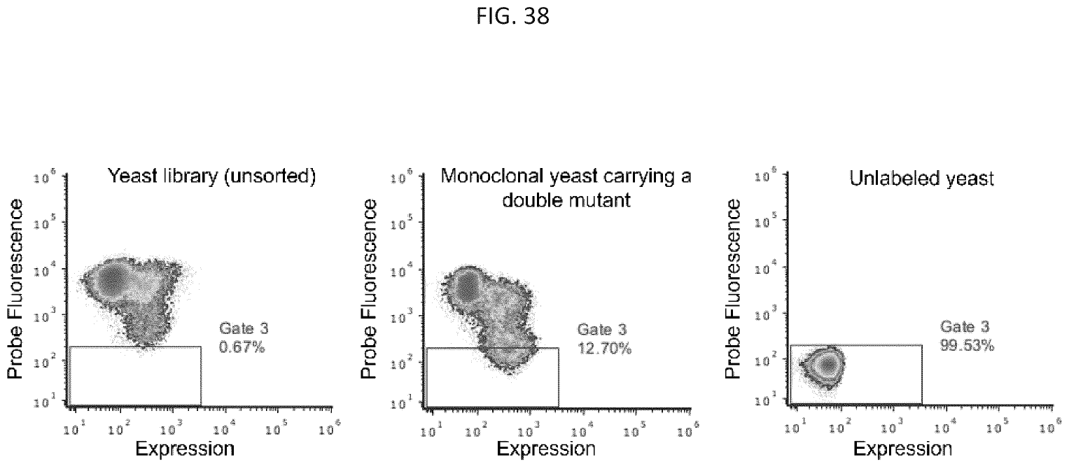

To pursue this proposal, we prepared an error-prone library of “ICCG+Y” mutants and subjected it to five rounds of cell sorting, in which the labeling and probe hydrolysis steps all occurred at acidic pH. After completing these four rounds of sorting, we sequenced the yeast DNA to assess which mutations were enriched, and the enriched mutants were transformed into yeast to create monoclonal populations for activity testing. FIG. 38 shows flow cytometry data on the increase in enzymatic activity observed by monoclonal yeast carrying a promising double mutant. A dramatic increase in activity was evident based on the lower y-axis fluorescence signal.

Importantly, we needed to investigate whether the improvements observed on the yeast surface in cleavage of the PET-mimicking probe at pH 4 would translate to improvement in PET film degradation. We cloned, overexpressed, and purified the mutants from bacteria, and then tested their ability to degrade PET films at pH 4. Promisingly, two of the mutants exhibited improvements in PET depolymerization at pH 4 relative to the parent ICCG+H218Y (FIG. 39), confirming the effectiveness of our ultrahigh-throughput platform. Given these promising findings, these mutants are being subjected to combined experimental and computational investigations to understand their mechanisms and identify which active site features influence acid sensitivity the most.

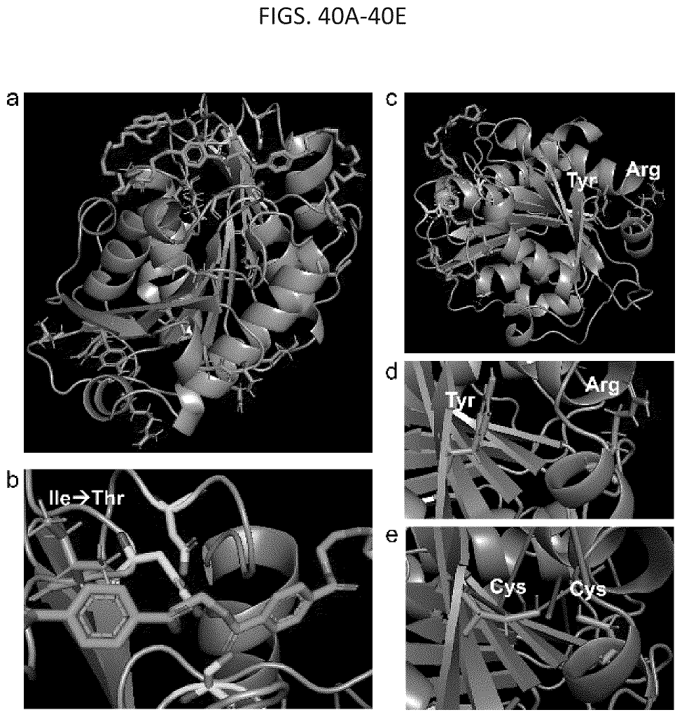

FIG. 40 depicts a structural model highlighting mutations that were identified using our platform for pH 4 evolution below. Mutations were identified throughout the ICCG+H218Y structure, including positions adjacent to the catalytic residues as well as several more distant mutations. In one example, a hydrophobic residue was mutated to a polar Thr residue immediately adjacent to the catalytic triad (FIG. 40B), suggesting a change in hydrogen-bonding with catalytic residues and/or solvent. In another example, a double mutant was identified in which two neighboring residues (Tyr and Arg) were both mutated to Cys, at a location distal from the catalytic residues (FIGS. 40C and D). This finding raises the possibility that a novel disulfide may have been introduced into this mutant (FIG. 40E), which we are investigating further.

### Second Generation Acid-Tolerant Platform

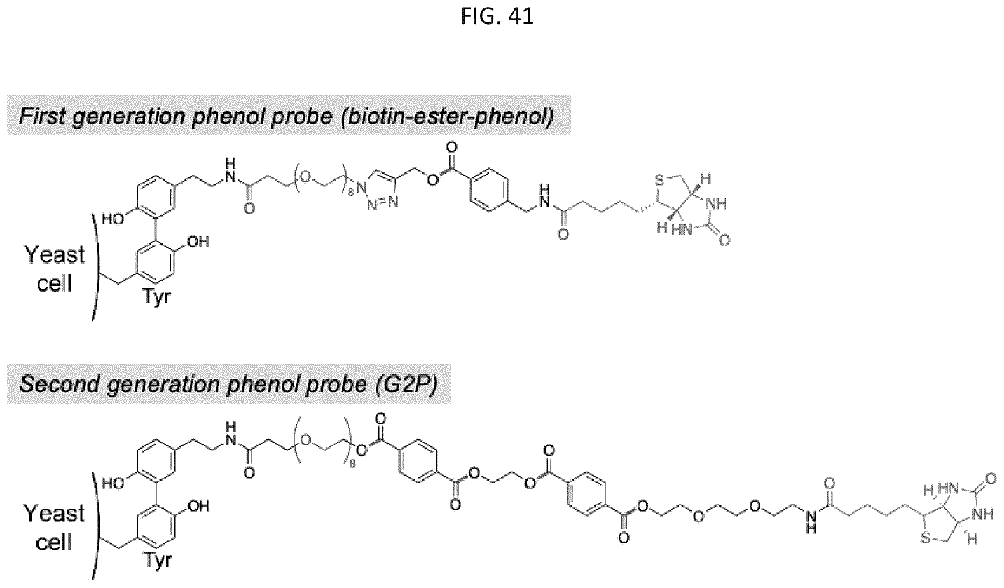

We sought to make further improvements to the platform to perform the whole selection at pH 4. We sought to improve the probe design further to include multiple cleavable aromatic ester bonds such that the portion of the probe in the active site of the enzyme could more closely model actual PET film by having two aromatic rings, as opposed to one in the first phenol probe. In the future, this probe design could be altered to test the importance of terminal ester bonds flanking the aromatic rings as these may be easier to cleave (FIG. 41).

### Materials and Methods

Materials used were the same as an Example 1, except methods of Example 5 also employed:

Citrate buffer pH 4 prepared as follows: 33.8 mM sodium citrate dihydrate and 66.2 mM citric acid monohydrate. For cell labeling and washing steps, citrate buffer pH 4 was supplemented with 1 mg/mL BSA, referred to as Cit-B. Citrate buffer pH 5 was prepared as follows: 58.1 mM sodium citrate dihydrate and 41.9 mM citric acid monohydrate.

Flow cytometry analysis was performed using an Attune NxT V6 Flow Cytometer (ThermoFisher Scientific) equipped with 488, 561, and 633 nm lasers and appropriate emission filters (530/30 for GFP (Gamillus), 585/16 for phycoerythrin, and 670/14 for AlexaFluor647). Cell sorting was performed using a FACSAria Cell Sorter (BD Biosciences) with 488, 561 and 633 nm lasers and corresponding filters (530/30 for Gamillus, 582/15 for phycoerythrin, 660/20 for AlexaFluor647).

Additional PET Hydrolysis experiments involved the use of 100 mM potassium phosphate buffer (kPi) pH 8, deionized water, or a 20 μM solution of terephthalic acid (TPA) in deionized water.

### General Labeling Protocol for Phenol-Based Probe Cell Labeling

Yeast were induced into SGCAA media as described above, then subjected to the following labeling protocol. The SGCAA culture was allowed to grow for 18 hours to saturation at 30° C. (or 24 hours for 20° C. expression of Gamillus yeast). The entire culture was spun down for 3 minutes at 4300×g at 24° C. The supernatant was discarded, and the cells were washed twice with PBS pH 7.4. The OD600 was measured to determine cell density. An aliquot of cells equal to at least ten-fold over coverage of library was removed and spun down. From this point forward, samples in 1.5 mL Eppendorf tubes were spun down for 1 minute at 10,000×g at 24° C. and samples in 5-, 15-, or 50-mL vessels were spun down for 3 minutes at 4300×g at 24° C.

For SpyTag cells: The cell pellet was suspended in a PBS-B pH 7.4 solution (50 μL per 60 million yeast) containing 1 μM final concentration of SpyCatcher-AlexaFluor 647 conjugate. The tube was placed horizontally on an orbital shaker with 180 rpm shaking for 45 minutes at room temperature followed by two PBS-B pH 7.4 washes (200 μL per 60 million yeast).

Biotin ester phenol or Gen2 Probe labeling: The cell pellet was suspended in a citrate pH 4 (for Gamillus yeast) or pH 5 (for SpyTag yeast) solution containing 10 μM horse radish peroxidase (HRP, Sigma), 50 μL per 60 million yeast. The pellet was thoroughly vortexed before addition of citrate buffer (at same pH as HRP solution) containing 2 mM H₂O₂ and 20 μM phenol probe (50 μL per 60 million yeast). The sample was immediately vortexed, initiating a one-minute reaction time with 180 rpm shaking followed by quenching with 100 mM sodium ascorbate (10 μL per 60 million yeast). The solution was then immediately diluted with cit-B pH 4 (1 mL per 60 million yeast). The sample was spun down and the supernatant was removed. The sample was then washed once with cit-B pH 4 and once with citrate buffer pH 4 (200 μL per 60 million yeast). The samples were then suspended in citrate buffer pH 4 (1 mL per 60 million yeast) for a 2-hour (biotin ester phenol probe) or 30-minute (Gen2 probe) incubation period. After the incubation period, the cells were spun down and the supernatant was removed. The samples were washed twice with cit-B pH 4 (200 μL per 60 million yeast).

Separately, a solution containing 1:00 streptavidin-phycoerythrin (sAv-PE, Invitrogen, S866) in cit-B pH 4 was prepared and added to the washed pellet (50 μL per 60 million yeast). The pellet was resuspended and placed horizontally on a rotating platform to label for 30 minutes at room temperature. The sample was spun down and the cells were washed twice with cit-B pH 4.

After removing the last wash, the cells were then resuspended in cit-B pH 4 to a density of approximately 60 million cell/mL and then passed through a 35 μm sterile filter. The solution was stored at 4° C. until it was analyzed on the FACS instrumentation. Gates were drawn to collect cells with positive AF647 (for SpyTag) or GFP (for Gamillus) and low in PE signal. Sorted cells were collected into a 15 mL culture tube coated with PBS-B and containing 1 mL of SDCAA media. Subsequently 9 mL of SDCAA was added as well as 1% penicillin-streptomycin, 50 μg/mL kanamycin and 10 μg/mL tetracycline. The cells were allowed to grow to saturation in SDCAA for 48 hours before being passaged for the next round of sorting.

### Synthesis

Overview of Synthetic Scheme is shown at Example 1.

### Synthesis of Tyramine PEG₈ Azide (4)

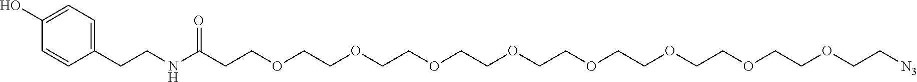 [SMILES: [N-]=[N+]=NCCOCCOCCOCCOCCOCCOCCOCCOCCC(=O)NCCc1ccc(O)cc1]

1-azido-N-(4-hydroxyphenethyl)-3,6,9,12,15,18,21,24-octaoxaheptacosan-27-amide (4). 4.5 mg of tyramine (1.11 eq, 0.032 mmol) was dissolved in a mixture of 300 μL of acetonitrile and azide PEG₈ NHS ester (16.7 mg, 1 eq, 0.029 mmol). While stirring at 600 rpm, N,N-Diisopropylethylamine (DIPEA, 69.6 mg, 18.2 eq, 0.539 mmol) was added. After two hours of stirring, 100 μL of anhydrous DMF was added to encourage solvation of reagents. The solution stirred for 18 hours overnight. The crude reaction mixture was concentrated via rotary evaporation, dissolved in 80 μL of 50/50 v/v water:MeCN, and purified via HPLC fraction collection. 10 separate injections of 95 μL of crude product mixture was injected and the product was collected from 4.4 to 5 minutes retention time using the method described in the HPLC analysis section above. The combined fractions were concentrated via rotary evaporation and further dried under high vacuum for 2 hours to a colorless oil residue (15.7 mg isolated, 0.027 mmol, 90.8% yield).

1H NMR (400 MHz, CDCl3) δ 7.07-6.99 (m, 2H), 6.87-6.80 (m, 2H), 6.64 (s, 1H), 3.63 (dh, J=10.6, 5.2 Hz, 26H), 3.55 (dd, J=6.1, 3.5 Hz, 2H), 3.53-3.44 (m, 4H), 3.42-3.34 (m, 4H), 2.73 (t, J=6.4 Hz, 2H), 2.45 (t, J=5.5 Hz, 2H).

### Synthesis of Biotin Ester Triazole Phenol (5)

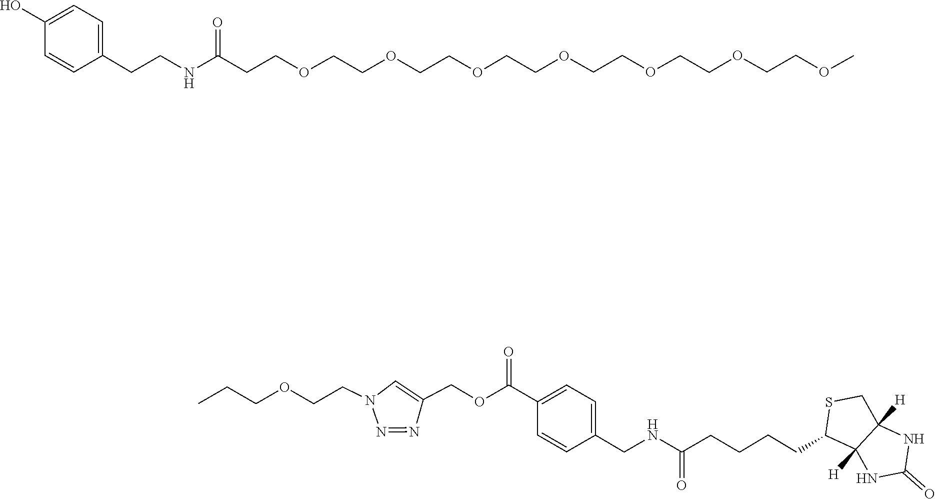

(1-(30-(4-hydroxyphenyl)-27-oxo-3,6,9,12,15,18,21,24-octaoxa-28-azatriacontyl)-1H-1,2,3-triazol-4-yl)methyl 4-((5-((3aS,4S,6aR)-2-oxohexahydro-1H-thieno[3,4-d]imidazol-4-yl)pentanamido)methyl)benzoate (5). To 12.5 mg of biotin aminomethylbenzoate (3) was added 77 μL of a solution containing 3 mg of TBTA and 0.33 mg of CuBr in 83% methanol, 17% DMSO. 200 μL of DMSO was then added to facilitate solvation of reagents. 15 mg of tyramine PEG₈ azide (4) was then added and stirred at 500 rpm. 1 mg of triethylamine was then added, and the reaction solution was placed under nitrogen and heated to 55° C. overnight for 18 hours. After cooling to room temperature, the crude reaction mixture was diluted ten-fold with 50% v/v water:MeCN. 19 separate injections of 95 μL of crude product mixture were injected and the product was collected from 3.23 to 3.45 minutes retention time using the following method with instrumentation and column described above: ultrapure water with 0.1% trifluoroacetic acid as mobile phase A and acetonitrile as mobile phase B (10-40% B over 1 minute, then 40-70% B over 3.4 minutes, then 70-90% B over 0.1 minutes. The gradients were followed by a 1.5-minute wash at 90% B and then re-equilibration at 10% B), with a column oven temperature of 30° C. The combined fractions were concentrated via rotary evaporation to a colorless oil residue (1.6 mg isolated, 0.027 mmol, 6.2% yield).

MS: [(M+2H)2+]calculated: 501.7463, found: 501.7458; [(M+H)+] calculated: 1002.4853, found: 1002.4840

1H NMR (400 MHz, DMSO) δ 9.17 (s, 1H), 8.41 (t, 1H), 8.21 (s, 1H), 7.90 (d, J=8.2 Hz, 3H), 7.86 (s, 1H), 7.37 (d, J=8.1 Hz, 2H), 6.98 (d, J=8.3 Hz, 2H), 6.67 (d, J=8.4 Hz, 2H), 6.43 (s, 1H), 6.36 (s, 1H), 5.38 (s, 2H), 4.54 (t, J=5.2 Hz, 2H), 4.40-4.25 (m, 3H), 4.12 (s, 1H), 3.82 (t, J=5.2 Hz, 2H), 3.69-3.43 (m, 28H), 3.18 (q, J=6.9 Hz, 3H), 3.09 (s, 1H), 2.82 (dd, J=12.5, 5.0 Hz, 1H), 2.57 (t, J=8.3 Hz, 4H), 2.28 (t, J=6.4 Hz, 2H), 2.16 (t, J=7.4 Hz, 2H), 1.72-1.17 (m, 6H).

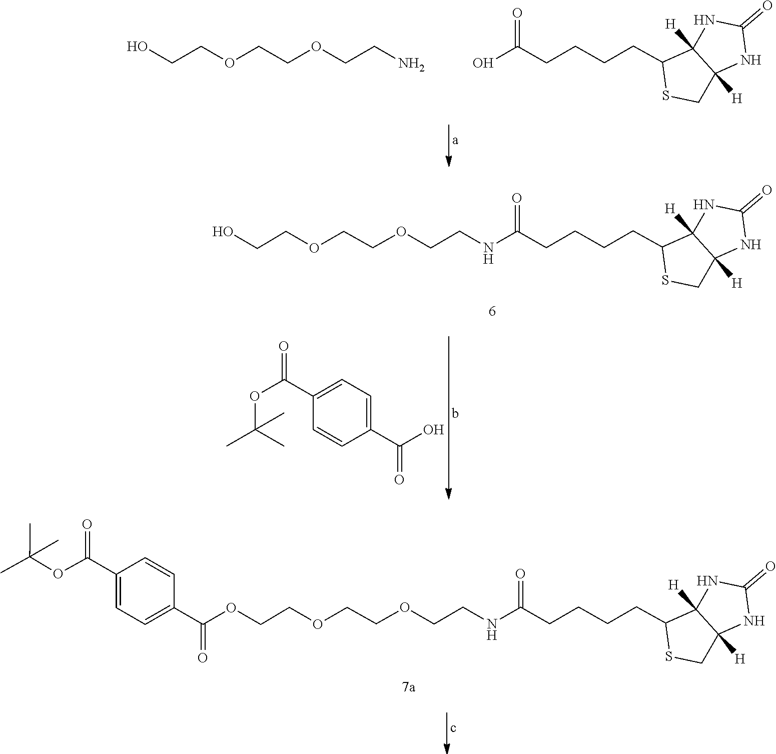 [SMILES: NCCOCCOCCO ; O=C(O)CCCCC1SC[C@H]2NC(=O)N[C@@H]12 ; O=C(CCCCC1SC[C@H]2NC(=O)N[C@@H]12)NCCOCCOCCO ; CC(C)(C)OC(=O)c1ccc(C(=O)O)cc1 ; CC(C)(C)OC(=O)c1ccc(C(=O)OCCOCCOCCNC(=O)CCCCC2SC[C@H]3NC(=O)N[C@@H]23)cc1]

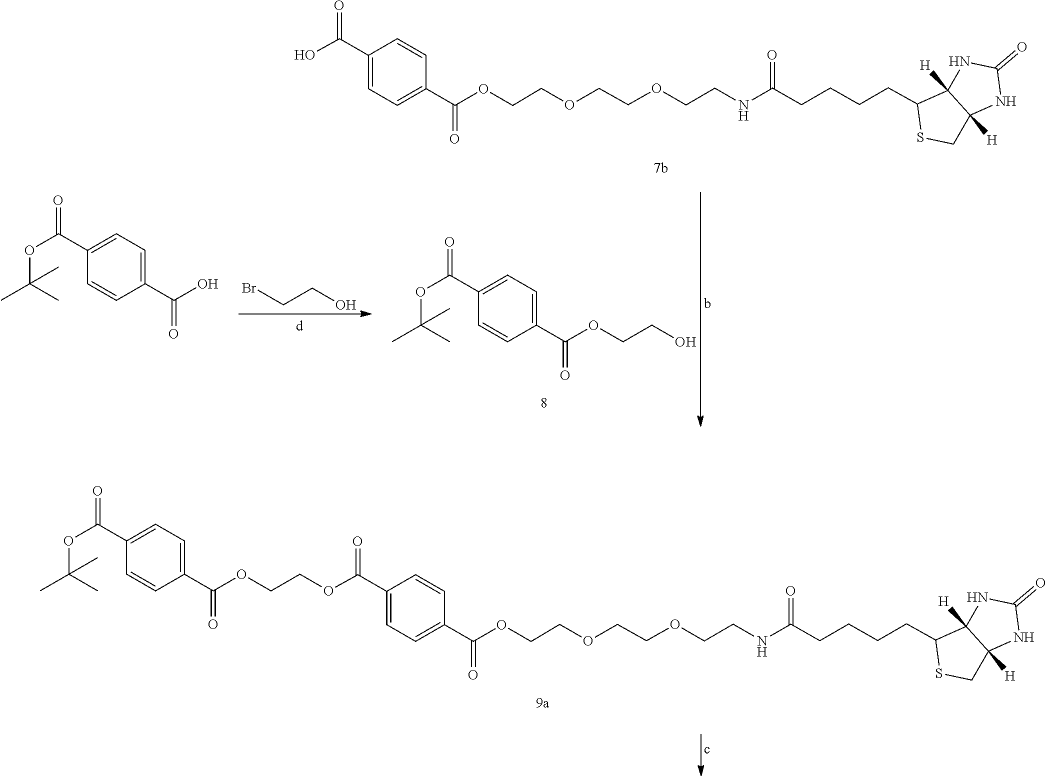

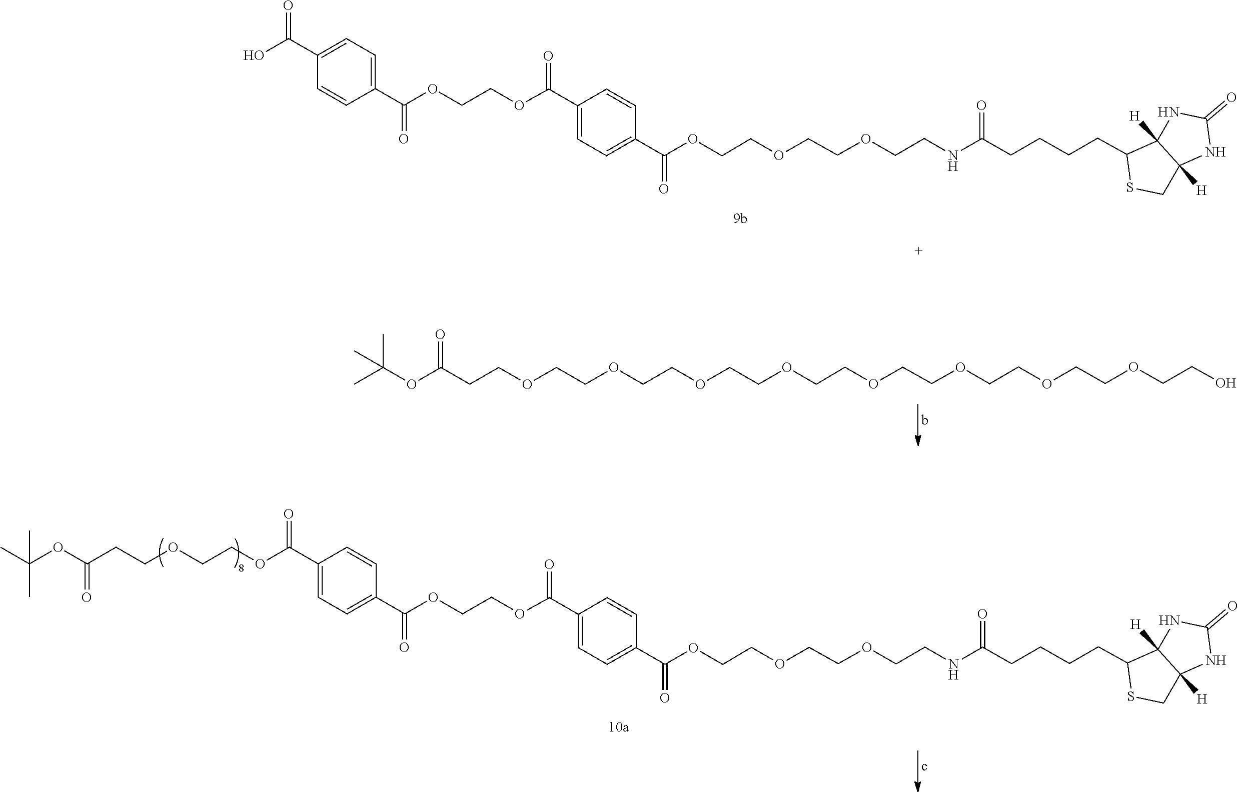

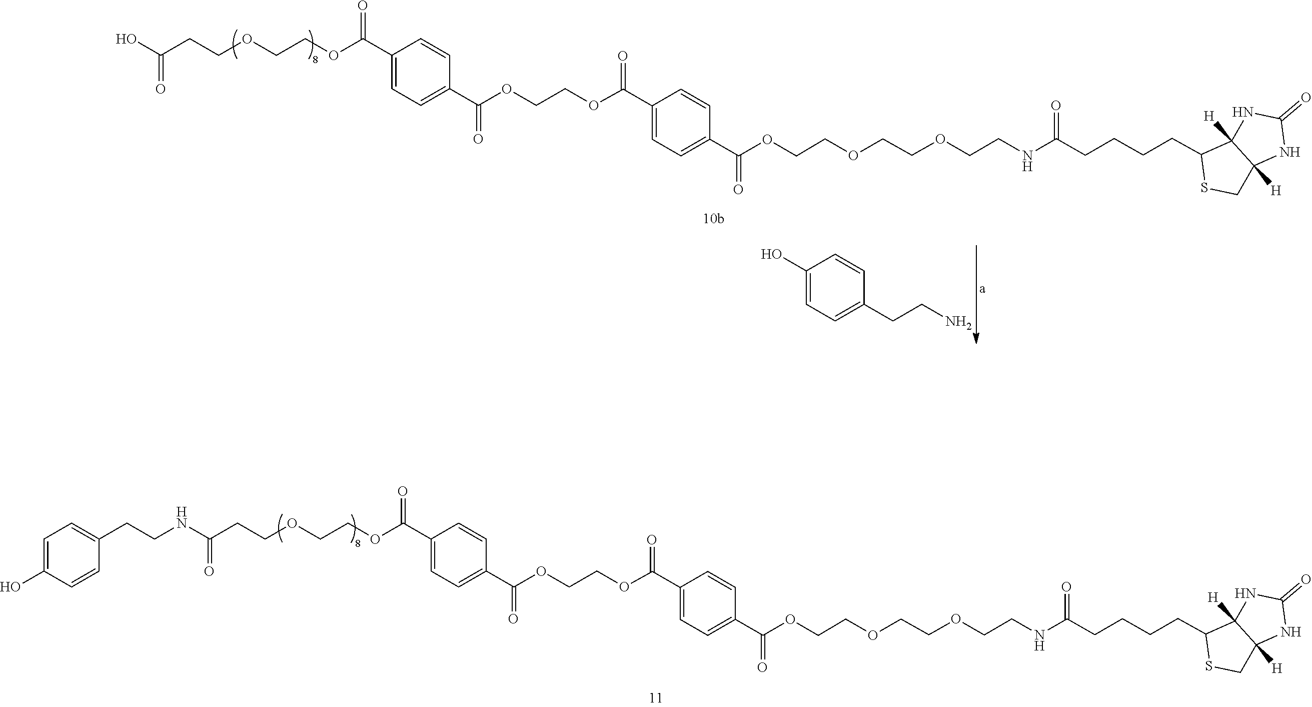

Synthetic scheme for synthesis of biotin PET₂ phenol (11). A. HBTU, TEA, DMA. B. EDC, DMAP, pyridine. C. Phosphoric acid in MeCN. D. DMF, 125° C.

### General Procedure A—Peptide Coupling

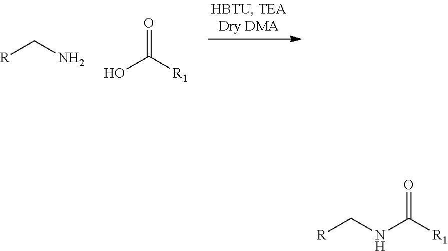

Carboxylic acid compound (1 eq.) was dissolved in dry dimethylacetamide (DMA) under nitrogen. The solution was cooled to 0° C. with an ice bath, then HBTU (1.3 eq.) was added, and the solution stirred on an ice bath for 15 minutes under nitrogen. Triethylamine (4 eq.) was then added followed by amine compound (1.1 eq.). The reaction solution stirred at 0° C.→room temperature for 18 hours. The reaction was worked up as indicated in individual syntheses.

Biotin PEG₃ alcohol (6)

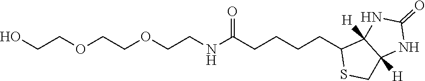 [SMILES: O=C(CCCCC1SC[C@H]2NC(=O)N[C@@H]12)NCCOCCOCCO]

N-(2-(2-(2-hydroxyethoxy)ethoxy)ethyl)-5-((3aS,6aR)-2-oxohexahydro-1H-thieno[3,4-d]imidazol-4-yl)pentanamide (6) was synthesized according to General Procedure A using 1 g of biotin (4.09 mmol) and 672 mg of amino-PEG₃-alcohol (4.5 mmol). The crude reaction mixture was dripped into 50 mL of diethyl ether and stirred for 30 minutes to afford a brown precipitate. The ether was decanted from the precipitate and the precipitate was sonicated in 50 mL of DCM for 15 minutes. The resultant white precipitate was isolated via vacuum filtration and washed twice with 15 mL of diethyl ether to afford a white powder (1.052 g, 2.94 mmol, 71.9% yield).

¹H NMR (400 MHz, DMSO) δ 7.84 (t, J=5.7 Hz, 1H), 6.42 (s, 1H), 6.36 (s, 1H), 4.60 (s, 1H), 4.30 (dd, J=7.7, 5.0 Hz, 1H), 4.12 (ddd, J=7.4, 4.5, 2.0 Hz, 1H), 3.50 (s, 4H), 3.40 (dt, J=8.8, 5.5 Hz, 6H), 3.22-3.13 (m, 2H), 3.13-3.04 (m, 1H), 2.82 (dd, J=12.4, 5.0 Hz, 1H), 2.57 (d, J=12.4 Hz, 1H), 2.06 (t, J=7.4 Hz, 2H), 1.67-1.21 (m, 6H).

### Biotin PET₂ Phenol (11)

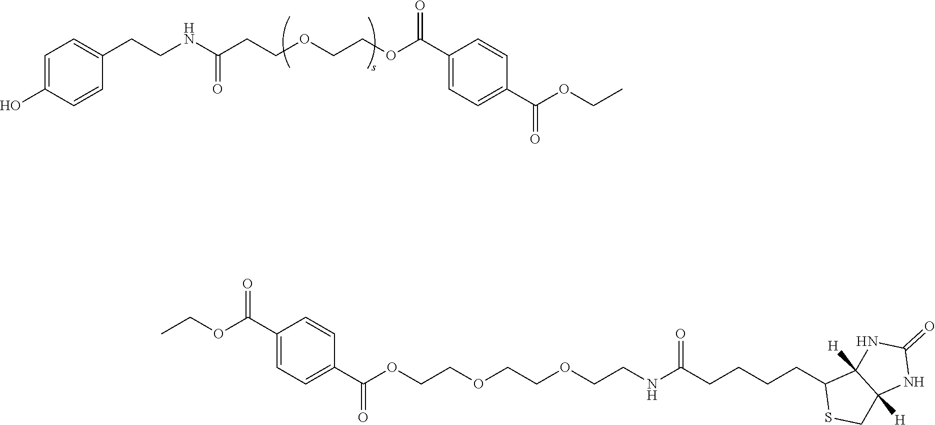

22-ethyl-28-(4-hydroxyphenyl)-25-oxo-3,6,9,12,15,18,21,22λ³-octaoxa-26-azaoctacosyl (2-((4-(12-oxo-16-((3aS,6aR)-2-oxohexahydro-1H-thieno[3,4-d]imidazol-4-yl)-2,5,8-trioxa-11-azahexadecanoyl)benzoyl)oxy)ethyl) terephthalate (11) was synthesized according to General Procedure A using 53 mg (0.05 mmol) of biotin PET₂ PEG₈ (10b), 34 mg (0.25 mmol, 5.3 eq.) of tyramine, 112 mg of HBTU (0.29 mmol, 6.3 eq.), and 94 mg of triethylamine (0.93 mmol, 20 eq). After 14 hours of stirring the crude reaction mixture was dripped into 10 mL of diethyl ether, and an orange oil crashed out of solution. The ether was decanted leaving behind the oil, and the oil was purified via HPLC fraction collection by solvation in 8 mL of 50% v/v water in acetonitrile followed by 60 separate injections of 95 μL of crude product mixture. The product was collected from 5.56 to 5.86 minutes retention time using the following method with instrumentation and column described above: ultrapure water with 0.1% trifluoroacetic acid as mobile phase A and acetonitrile as mobile phase B (10-60% B over 4.4 minutes, then 60-90% B over 0.1 minutes. The gradients were followed by a 1.5-minute wash at 90% B and then re-equilibration at 10% B), with a column oven temperature of 30° C. The combined fractions were concentrated via rotary evaporation to a colorless oil residue (10 mg isolated from ¾ of the crude reaction mixture, 0.008 mmol, 21.3% yield on amount purified). MS: [(M+H)+]calculated: 1259.5527, found: 1259.5524.

¹H NMR (400 MHz, CD₃CN) δ 8.12 (d, J=2.0 Hz, 8H), 7.05 (d, J=8.4 Hz, 2H), 6.79-6.69 (m, 2H), 6.65 (s, 1H), 6.49 (s, 1H), 5.28 (s, 1H), 5.06 (s, 1H), 4.70 (s, 4H), 4.50-4.44 (m, 4H), 4.41 (d, J=4.9 Hz, 1H), 4.24 (t, J=6.1 Hz, 1H), 3.82 (dt, J=6.8, 2.6 Hz, 4H), 3.66 (dd, J=5.9, 3.2 Hz, 5H), 3.63-3.50 (m, 28H), 3.48 (t, J=5.6 Hz, 2H), 3.34 (q, J=6.8 Hz, 2H), 3.28 (q, J=5.6 Hz, 2H), 3.19-3.08 (m, 1H), 2.89 (dd, J=12.8, 5.0 Hz, 1H), 2.69-2.64 (m, 2H), 2.34 (t, J=5.8 Hz, 2H), 2.15-2.06 (m, 4H), 1.72-1.30 (m, 6H).

### General Procedure B—Ester Bond Formation

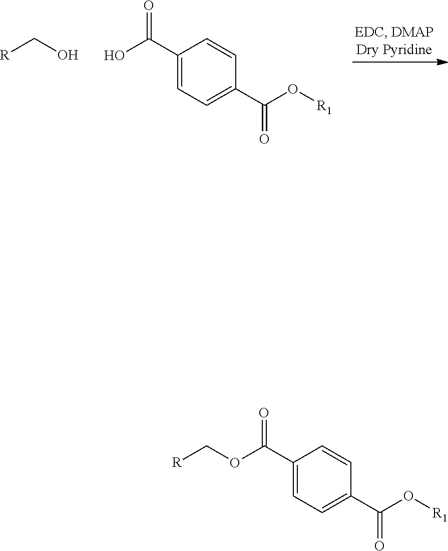

Alcohol compound (1 eq.) was dissolved in dry pyridine under nitrogen. EDC (1.05 eq.) followed by DMAP (0.25 eq.) and carboxylic acid compound (1 eq.) were then added. The reaction solution stirred under nitrogen at room temperature for 18 hours. The reaction was worked up as indicated in individual syntheses.

### T-Butyl Biotin TPA (7a)

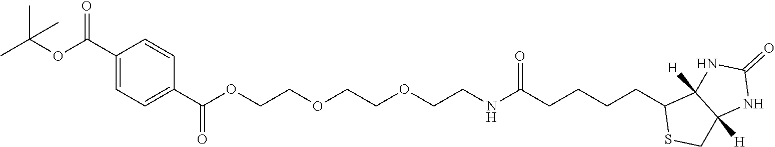 [SMILES: CC(C)(C)OC(=O)c1ccc(C(=O)OCCOCCOCCNC(=O)CCCCC2SC[C@H]3NC(=O)N[C@@H]23)cc1]

tert-butyl(2-(2-(2-(5-((3aS,6aR)-2-oxohexahydro-1H-thieno[3,4-d]imidazol-4 yl)pentanamido) ethoxy)ethoxy)ethyl) terephthalate (7a) was synthesized according to General Procedure B using 426.3 mg (1.1 mmol) of Biotin PEG₃ alcohol (6) and 252.3 mg (1.1 mmol) of 4-(tert-butoxycarbonyl)benzoic acid. The reaction mixture was dripped into 20 mL of diethyl ether then 20 mL of saturated sodium bicarbonate was added. The ether layer was isolated, and the aqueous layer was extracted once with 5 mL diethyl ether then twice with 5 mL of ethyl acetate. The combined organic layers were concentrated via rotary evaporation to afford a white solid (420 mg, 0.85 mmol, 77.3% yield).

¹H NMR (400 MHz, DMSO) δ 8.09-7.99 (m, 4H), 7.86-7.75 (m, 1H), 6.41 (s, 1H), 6.35 (s, 1H), 4.47-4.37 (m, 2H), 4.32-4.25 (m, 1H), 4.11 (ddd, J=7.5, 4.6, 1.8 Hz, 1H), 3.81-3.72 (m, 2H), 3.59 (dd, J=5.9, 3.5 Hz, 2H), 3.52 (dd, J=5.9, 3.5 Hz, 2H), 3.40 (d, J=6.0 Hz, 2H), 3.16 (q, J=5.9 Hz, 2H), 3.08 (ddd, J=10.5, 8.6, 5.0 Hz, 1H), 2.80 (dd, J=12.5, 5.1 Hz, 1H), 2.56 (d, J=16.9 Hz, 1H), 2.04 (t, J=7.4 Hz, 2H), 1.56 (s, 9H), 1.52-1.19 (m, 6H).

### T-Butyl Biotin PET₂ (9a)

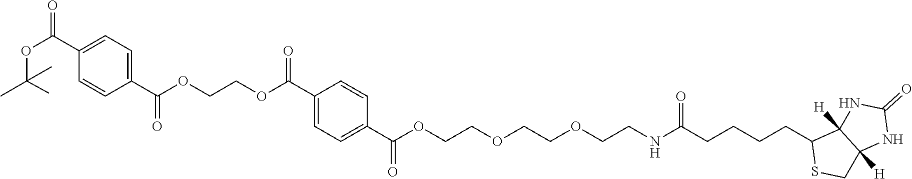 [SMILES: CC(C)(C)OC(=O)c1ccc(C(=O)OCCOC(=O)c2ccc(C(=O)OCCOCCOCCNC(=O)CCCCC3SC[C@H]4NC(=O)N[C@@H]34)cc2)cc1]

2-((4-(tert-butoxycarbonyl)benzoyl)oxy)ethyl (2-(2-(2-(5-((3aS,6aR)-2-oxohexahydro-1H-thieno[3,4-d]imidazol-4-yl)pentanamido)ethoxy)ethoxy)ethyl) terephthalate (9a) was synthesized according to General Procedure B using 220 mg (0.42 mmol) of biotin TPA (7b), 134.8 mg (0.5 mmol) of T-butyl MHET (8), 15.5 mg (0.12 mmol) of DMAP, and 82.5 mg (0.53 mmol) of EDC. The reaction mixture was dripped into 20 mL of diethyl ether and the resultant precipitate was isolated via vacuum filtration and washed twice with 5 mL of saturated sodium bicarbonate solution followed by 10 mL of diethyl ether. The precipitate was then dissolved in acetonitrile and the soluble portion was concentrated via rotary evaporation to afford an off-white solid (263 mg, 0.34 mmol, 81.1% yield).

¹H NMR (400 MHz, DMSO) δ 8.19-7.95 (m, 8H), 7.81 (t, J=5.5 Hz, 1H), 6.40 (s, 1H), 6.35 (s, 1H), 4.68 (s, 3H), 4.41 (dd, J=5.8, 3.5 Hz, 2H), 4.29 (t, J=6.4 Hz, 1H), 4.11 (ddd, J=7.5, 4.6, 1.9 Hz, 1H), 3.80-3.72 (m, 2H), 3.59 (dq, J=6.8, 3.7 Hz, 2H), 3.51 (dd, J=5.9, 3.4 Hz, 3H), 3.38 (d, J=5.8 Hz, 2H), 3.16 (q, J=5.8 Hz, 2H), 3.11-3.05 (m, 1H), 2.83-2.72 (m, 2H), 2.06-2.00 (m, 2H), 1.56 (d, J=4.2 Hz, 9H), 1.52-1.15 (m, 6H).

T-Butyl Biotin PET₂ PEG₈ (10a)

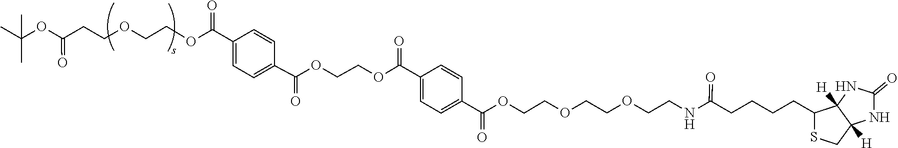

22-ethyl-27,27-dimethyl-25-oxo-3,6,9,12,15,18,21,22λ³,26-nonaoxaoctacosyl (2-((4-(12-oxo-16-((3aS,6aR)-2-oxohexahydro-1H-thieno[3,4-d]imidazol-4-yl)-2,5,8-trioxa-11-azahexadecanoyl)benzoyl)oxy)ethyl) terephthalate (10a) was synthesized according to General Procedure B using 128.9 mg (0.18 mmol) of biotin PET₂ (9b) and 89.9 mg (0.18 mmol) of tert-butyl 1-hydroxy-3,6,9,12,15,18,21,24-octaoxaheptacosan-27-oate. 10 mL of diethyl ether was added to the crude reaction mixture, causing a precipitate to crash out. The ether layer was decanted and concentrated via rotary evaporation and deprotection following General Procedure C was carried out without characterization of intermediate 10a.

### General Procedure C—T-Butyl Deprotection of Carboxylic Acids

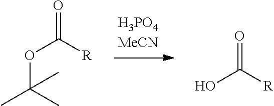

Tert-butyl protected carboxylic acid was dissolved in 200 μL of acetonitrile per 100 mg of t-butyl material followed by addition of 0.5 eq by volume of phosphoric acid (85% wt. % in water). The reaction stirred overnight for 18 hours. See individual syntheses below for work up protocols.

### Biotin TPA (7b)

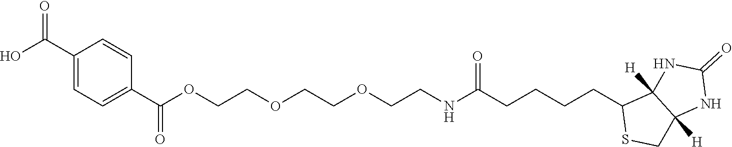 [SMILES: O=C(CCCCC1SC[C@H]2NC(=O)N[C@@H]12)NCCOCCOCCOC(=O)c1ccc(C(=O)O)cc1]

4-(12-oxo-16-((3aS,6aR)-2-oxohexahydro-1H-thieno[3,4-d]imidazol-4-yl)-2,5,8-trioxa-11-azahexadecanoyl)benzoic acid (7b) was synthesized according to General Procedure C using 420 mg (0.85 mmol) of T-butyl biotin TPA (7a), 800 μL of acetonitrile and 400 μL phosphoric acid. After overnight reaction 10 mL of deionized water was added and stirred for 15 minutes to precipitate out the product which was isolated via vacuum filtration as an off-white solid (282 mg, 0.54 mmol, 63.5% yield). MS: [(M+H)+]calculated: 524.2061, found: 524.2060.

¹H NMR (400 MHz, DMSO) δ 8.05 (d, J=12.4 Hz, 4H), 7.81 (t, J=5.7 Hz, 1H), 6.41 (s, 1H), 6.36 (s, 1H), 4.49-4.39 (m, 2H), 4.29 (dd, J=7.9, 5.0 Hz, 1H), 4.11 (t, J=6.4 Hz, 1H), 3.76 (t, J=4.7 Hz, 2H), 3.59 (dd, J=5.9, 3.5 Hz, 2H), 3.52 (dd, J=5.9, 3.5 Hz, 2H), 3.38 (d, J=5.9 Hz, 2H), 3.16 (q, J=5.9 Hz, 2H), 3.07 (dt, J=10.4, 5.3 Hz, 1H), 2.81 (dd, J=12.4, 5.1 Hz, 1H), 2.56 (d, J=16.9 Hz, 1H), 2.05 (dd, J=13.2, 5.9 Hz, 2H), 1.53-1.16 (m, 6H).

### Biotin PET₂ (9b)

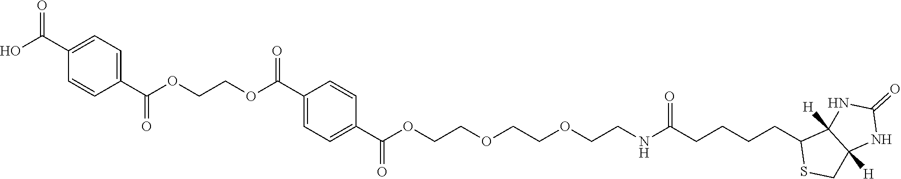 [SMILES: O=C(CCCCC1SC[C@H]2NC(=O)N[C@@H]12)NCCOCCOCCOC(=O)c1ccc(C(=O)OCCOC(=O)c2ccc(C(=O)O)cc2)cc1]

4-((2-((4-(12-oxo-16-((3aS,6aR)-2-oxohexahydro-1H-thieno[3,4-d]imidazol-4-yl)-2,5,8-trioxa-11-azahexadecanoyl)benzoyl)oxy)ethoxy)carbonyl)benzoic acid (9b) was synthesized according to General Procedure C using 263 mg (0.34 mmol) of t-butyl biotin PET₂ (9a), 600 μL of acetonitrile and 400 μL of phosphoric acid. After stirring for 18 hours, 10 mL of deionized water was added to the vial and stirred for 30 minutes, after which a precipitate formed that was isolated via vacuum filtration and washed with 5 mL of deionized water, 10 mL of diethyl ether, and 10 mL of acetonitrile. The precipitate was dried in a 60° C. oven for 12 hours to afford a white solid (128.2 mg, 0.18 mmol, 52.9% yield). MS: [(M+H)+]calculated: 716.2484, found: 716.2484.

¹H NMR (400 MHz, DMSO) δ 8.17-7.98 (m, 8H), 7.81 (t, J=5.5 Hz, 1H), 6.41 (s, 1H), 6.35 (s, 1H), 4.68 (s, 3H), 4.42 (t, J=4.6 Hz, 2H), 4.29 (t, J=6.4 Hz, 1H), 4.11 (t, J=6.5 Hz, 1H), 3.75 (t, J=4.6 Hz, 2H), 3.59 (dd, J=5.8, 3.4 Hz, 2H), 3.52 (dt, J=6.7, 3.8 Hz, 2H), 3.16 (q, J=5.8 Hz, 3H), 3.08 (d, J=10.1 Hz, 2H), 2.80 (dd, J=12.6, 5.0 Hz, 1H), 2.58 (s, 1H), 2.04 (t, J=7.4 Hz, 2H), 1.67-1.16 (m, 7H).

Biotin PET₂ PEG₈ Acid (10b)

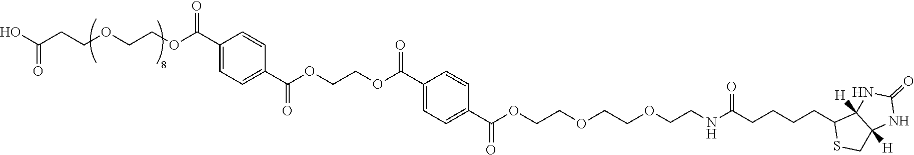

24-ethyl-1-oxo-1-(4-((2-((4-(12-oxo-16-((3aS,6aR)-2-oxohexahydro-1H-thieno[3,4-d]imidazol-4-yl)-2,5,8-trioxa-11-azahexadecanoyl)benzoyl)oxy)ethoxy)carbonyl)phenyl)-2,5,8,11,14,17,20,23,24λ³-nonaoxaheptacosan-27-oic acid (10b) was synthesized according to General Procedure C using t-butyl biotin PET₂ PEG₈ as soluble ether portion from preceding reaction. Assuming 100% retention of product, 0.18 mmol, approximately 215 mg, was used as the starting material. 800 μL of acetonitrile followed by 400 μL of phosphoric acid was added and stirred for 18 hours. 10 mL of deionized water was added and stirred for 30 minutes after which a precipitate was isolated via vacuum filtration. The precipitate was then washed with acetonitrile and the filtrate was concentrated via rotary evaporation to afford a colorless oil residue (53.2 mg, 0.05 mmol, 27.8% yield).

¹H NMR (400 MHz, CD₃CN) δ 8.11 (dd, J=5.6, 2.5 Hz, 8H), 6.57 (s, 1H), 4.68 (d, J=1.3 Hz, 4H), 4.50-4.39 (m, 5H), 4.26 (dd, J=7.8, 4.5 Hz, 1H), 3.85-3.75 (m, 4H), 3.70-3.62 (m, 5H), 3.58 (ddd, J=6.7, 3.4, 1.1 Hz, 4H), 3.53 (d, J=7.6 Hz, 28H), 3.46 (q, J=6.3 Hz, 2H), 3.31-3.22 (m, 2H), 3.20-3.09 (m, 1H), 2.88 (dd, J=12.8, 4.9 Hz, 1H), 2.14 (d, J=7.6 Hz, 2H), 1.71-1.22 (m, 6H).

Procedure D: Bromoethanol t-Butyl TPA Coupling

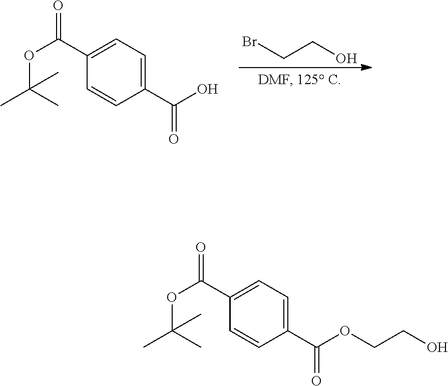

tert-butyl (2-hydroxyethyl) terephthalate (8). 93.1 mg (0.75 mmol, 1.2 eq.) of 2-bromoethanol followed by 104.3 mg (1.24 mmol, 2 eq.) NaHCO₃ was added to a 6-dram vial containing 138 mg (0.62 mmol, 1 eq.) of 4-(tert-butoxycarbonyl)benzoic acid dissolved in 2 mL of DMF while stirring at 300 rpm. The reaction was heated at 125° C. for 18 hours. After cooling to room temperature, 10 mL of deionized water was added, and the aqueous layer was extracted 3 times with 10 mL ethyl acetate. The combined organic layers were then washed with 10 mL of saturated sodium bicarbonate solution. The organic layer was concentrated via rotary evaporation to a yellow oil (152.9 mg, 0.57 mmol, 91.9% yield). MS: [(M+Na)+]calculated: 289.1046, found: 289.1044.

¹H NMR (400 MHz, CD₃CN) δ8.20-7.93 (m, 4H), 4.35 (t, J=4.8 Hz, 2H), 3.89-3.72 (m, 2H), 1.58 (s, 9H).

### REFERENCES

(1) Zakeri, B.; Fierer, J. O.; Celik, E.; Chittock, E. C.; Schwarz-Linek, U.; Moy, V. T.; Howarth, M. Peptide Tag Forming a Rapid Covalent Bond to a Protein, through Engineering a Bacterial Adhesin. Proceedings of the National Academy of Sciences 2012, 109 (12), E690-E697. https://doi.org/10.1073/pnas.1115485109.

(2) Pirillo, V.; Orlando, M.; Battaglia, C.; Pollegioni, L.; Molla, G. Efficient Polyethylene Terephthalate Degradation at Moderate Temperature: A Protein Engineering Study of LC-Cutinase Highlights the Key Role of Residue 243. The FEBS Journal 2023, 290 (12), 3185-3202. https://doi.org/10.1111/febs.16736.

What is claimed is:

1. A cell comprising:
a cell anchoring protein associated with an outer surface of the cell;
a nucleic acid sequence encoding a fusion protein within the cell, wherein the fusion protein comprises a candidate polymer-degrading enzyme and an anchoring sequence capable of associating with the cell anchoring protein; and
a probe associated with the outer surface of the cell, wherein the probe comprises a polymer substrate and a detectable probe label.

2. The cell of claim 1, wherein the fusion protein further comprises a detectable fusion protein label.

3. The cell of claim 2, wherein the detectable fusion protein label is a myc tag, a spy tag, a snap tag, or a halo tag.

4. The cell of claim 1, further comprising the fusion protein, wherein the anchoring sequence is associated with the cell anchoring protein.

5. The cell of claim 1, wherein the probe is associated with a primary amine or a phenol on the outer surface of the cell.

6. The cell of claim 5, wherein the polymer substrate is positioned between a linker associated with the primary amine or the phenol and the detectable probe label.

7. The cell of claim 1, wherein the candidate polymer-degrading enzyme is leaf and branch compost cutinase (LCC) comprising an amino acid sequence having at least 90% identity to SEQ ID NO: 1.

8. The cell of claim 7, wherein the LCC comprises a H218 mutation.

9. The cell of claim 8, wherein the LCC comprises a H218Y, H218R, H218L, or H218N mutation.

10. The cell of claim 7, wherein the LCC comprises a Y61C mutation and a R151C mutation.

11. The cell of claim 1, wherein the polymer substrate comprises an ester bond.

12. The cell of claim 11, wherein the polymer substrate is selected from an aromatic ester substrate, a lactic acid substrate, a polyamide substrate, a polyurethane substrate, and a polycarbonate substrate.

13. The cell of claim 1, wherein the cell anchoring protein is an Aga1P protein or a fragment thereof and the anchoring sequence is an Aga2P protein or a fragment thereof.

14. The cell of claim 1, wherein the cell is a yeast cell.

15. A composition comprising the cell of claim 1 and a first signal generator capable of binding with the detectable probe label.

16. The composition of claim 15, wherein the composition further comprises a second signal generator capable of binding with the detectable fusion protein label.

17. The composition of claim 15, further comprising an acidic media surrounding the cell.

18. The composition of claim 17, wherein the acidic media has a pH less than 4.

19. The composition of claim 15, wherein the composition comprises a heterogenous population of the cell, wherein the nucleic acid sequences encoding the fusion protein is different in each cell of the population; and wherein each fusion protein comprises a different candidate polymer-degrading enzyme.

20. A method comprising:
associating a probe with an outer surface of each of a plurality of cells, each probe comprising a polymer substrate and a detectable probe label,
wherein each cell comprises:
an cell anchoring protein associated with the outer surface of the cell, and
a fusion protein comprising an anchoring sequence and a candidate polymer-degrading enzyme associated with the cell anchoring protein, wherein each cell comprises a different candidate polymer-degrading enzyme; and
contacting the cells with a first signal generator capable of binding the detectable probe label;
removing unbound first signal generator; and
detecting a probe signal from the signal generator, wherein a reduced probe signal compared to a control probe signal from a cell that does not express the fusion protein indicates that the candidate polymer-degrading enzyme has polymer degrading activity.

21. The method of claim 20, wherein the fusion protein further comprises a detectable fusion protein label and the method further comprises:
contacting the cells with a second signal generator capable of binding with the detectable fusion protein label;
removing unbound second signal generator; and
detecting a fusion protein signal from the second signal generator, wherein detected fusion protein signal indicates that the cell expresses the fusion protein.

22. The method of claim 21, further comprising isolating cells having reduced probe signal compared to the control probe signal.

23. The method of claim 20, further comprising sequencing the fusion protein.
